(19) United States
(12) Patent Application Publication
(10) Pub. No.: US 2004/0014158 A1
(43) Pub. Date: Jan. 22, 2004

(54) Protein conjugates, methods, vectors, proteins and DNA for producing them, their use, and medicaments and vaccines containing a certain quantity of said protein conjugates

(76) Inventors: Adelbert Bacher, Garching (DE); Markus Fischer, Munchen (DE)

Correspondence Address: MYERS BIGEL SIBLEY & SAJOVEC, PO BOX 37428, RALEIGH, NC, 27627, US

(21) Appl. No.: 10/385,415

(22) Filed: Mar. 10, 2003

Related U.S. Application Data
(63) Continuation of application No. 09/936,028, filed on Jan. 31, 2002, now abandoned.
Application No. 09/936,028 is a 371 of international application No. PCT/EP00/01899, filed on Mar. 3, 2000.

(30) Foreign Application Priority Data
Mar. 8, 1999 (DE) 19910102.7

(51) Int. Cl. C12Q 1/66; C12N 9/06; C12N 1/21

(52) U.S. Cl. 435/8; 435/191; 435/252.31

(57) ABSTRACT

The invention relates to protein conjugates, methods, vectors, proteins and DNA for producing them, their use, and medicaments and vaccines containing a certain quantity of said protein conjugates. According to the invention, supramolecular particles are produced that represent one or more different, randomly selectable structural units in a large number on the surface of an individual, approximately spherical protein molecule. Icosahedral lumazine synthases are used as carrier proteins for peptides or proteins. A DNA fragment that encodes a peptide molecule is fused with a DNA fragment that encodes an icosahedral lumazine synthase by molecular-biological methods. Said DNA fragment is inserted into a cloning vector and transformed with an appropriate host strain. A polypeptide is expressed by gene expression. If certain peptide structures are used as the fusion partners, a post-translational change of said structures can be observed in the host strain. The chimeric peptide is purified and chemically modified if necessary. It is possible to produce icosahedral molecules that contain up to 120 different peptide motifs on their surfaces by mixing. The compounds produced lend themselves as auxiliary agents for carrying out analytical methods (ELISA, biosensors) or for producing vaccines.

### DESCRIPTION

Protein conjugates, procedures, vectors, proteins and DNA for their preparation, and their utilization as well as pharmaceutical agents and vaccines containing any of those.

The invention concerns protein conjugates, procedures, vectors, proteins and DNA for their preparation, and their utilization as well as pharmaceutical agents or vaccines containing any of those. The present invention serves for the preparation of supramolecular particles which display one or several different, arbitrarily selected structural units in large numbers on the surface of a single, approximately spherical protein molecule.

Properties of Lumazine Synthase and of Lumazine-Synthase-Based Artificial Protein Conjugates

6,7-Dimethyl-8-ribityllumazine synthase (subsequently designated lumazine synthase) catalyzes the penultimate step of vitamin B₂ biosynthesis in microorganisms and plants. Lumazine synthases from certain bacteria (e.g. Escherichia coli, Bacillus subtilis, Aquifex aeolicus) represent highly symmetrical, icosahedral complexes of 60 subunits with a molecular weight of approximately 1 MDalton (Bacher and Ladenstein, 1991; Bacher et al., 1980; Ladenstein et al., 1986, 1988, 1994; Mörtl et al., 1996). X-rays structures of the envelope capsid of lumazine synthase of Bacillus subtilis are known (Ladenstein et al., 1988, 1994; Ritsert et al., 1995). The protein of Bacillus subtilis can be denatured by the use of urea and can be subsequently renaturated. The efficacy of renaturation can be enhanced by the addition of a ligand (substrate analog), e.g. 5-nitro-6-ribitylamino-2,4(1H,3H)-pyrimidinedione or 5-nitroso-6-ribitylamino-2,4(1H,3)-pyrimidinedione. The fold of the renaturated protein is identical with the fold of the native lumazine synthase. In the presence of the said ligand, the lumazine synthase of Bacillus subtilis is stable up to a pH of 10. The environment of the inhibitor molecule is known on basis of the X-ray structure. The binding site of this ligand is formed by segments of adjacent monomers (Bacher et al., 1986; Ritsert et al., 1995). This constellation explains the supportive influence of the ligand during the renaturation of the high molecular weight protein complexes.

Lumazine synthases from different microorganisms can be expressed efficiently in recombinant strains of Escherichia coli and Bacillus subtilis. The recombinant proteins can be isolated in high yield.

The N-terminus as well as the C-terminus are located at the surface of the icosahedral capsid molecule. For the lumazine synthase of Bacillus subtilis, this was documented for the first time by X-ray structure analysis (Ladenstein et al., 1988). Using DNA synthesis, it was possible to obtain a gene for the expression of the thermostable lumazine synthase of the hyperthermophilic microorganism, Aquifex aeolicus, which is optimally adapted for the codon usage of Escherichia coli. The protein can be obtained in high amounts in recombinant form. At a temperature of 80° C., it is stable for at least one week. The conclusion that the structural relationships are the same in lumazine synthases of Aquifex aeolicus and Bacillus subtilis can be derived from the fact that fusion proteins with an elongation of the C-terminal and/or N-terminal end associate under formation of icosahedral capsids and from the observation that chimeric proteins consisting of parts of lumazine synthases from Aquifex aeolicus and Bacillus subtilis can be prepared. Consequently, it can be assumed that the quaternary structures of the enzymes are highly similar.

Icosahedral lumazine synthases can be functionalyzed at their surface by structural units. Oligopeptides or polypeptides whose segments can be arbitrarily determined are considered preferentially as structural units (biomolecules). The displayed proteins (conjugated biomolecules) are covalently linked with the carrier protein (lumazine synthase conjugate). A carrier protein is here defined, according to the invention, as a natural (unmodified) or a modified lumazine synthase whose primary structure has been modified. In that case, one or several amino acids can be replaced and/or removed and/or added and/or modified. The conservation of the original catalytic activity of the lumazine synthase is hereby not required. On the contrary, it is possible to use catalytically inactive, modified proteins for all applications according to the invention.

The number of conjugated biomolecules on the surface of the carrier protein can extend over a wide range, whereby, according to the invention, the surface can be decorated with up to 60 (at one terminus) respectively 120 (at both termini) respectively 180 (at both termini plus loop insertion) identical or structurally different peptide motifs. Protein subunits on the structural basis of lumazine synthase can also be assembled to even larger, approximately spherical particles as well as tubular structures. These associates can contain well about 60 subunits. They do however not possess the strict, geometric regularity of icosahedral, 60-meric lumazine synthase molecules.

The length of the peptide segments can vary over a wide range, according to the invention, preferentially between 1-500 amino acid residues, whereby the the peptide motifs can be present in unmodified as well as modified form.

Proteins, according to the invention, can also contain one or several amino acid analogs, or non-natural amino acids which can be introduced into the sequence by biological methods (e.g. by suppressor tRNA techniques, etc.) or by chemical methods (e.g. by coupling reagents, etc.). Moreover, modifications (e.g. glycosidation etc.) or derivatization (e.g. biotinylation etc.) can be present.

The respective genetic information for the specification of peptide segments which have been artificially introduced in the structure of lumazine synthase can range from few codons up to several genes, depending on whether an oligopeptide, a polypeptide or protein consisting of several subunits is intended to be it specified.

The surface of a lumazine synthase can also be modified chemically in such a way that the outer molecular periphery is covalently linked with a multiplicity of functional regions.

The production of hetero-oligomeric lumazine synthase conjugates proceeds via a dissociation step and a subsequent folding/reassociation step. The proteins which are present in monomeric form after denaturation can be mixed ad libitum. Since each of the recombinant subunits contains one respective constant lumazine synthase part, the renaturation of the lumazine synthase core structure is possible under formation of the natural icosahedral structure.

Immunological Analysis Methods Based on ELISA Assay Systems

Antibodies bind with high specificity to certain target structures (antigens). Assay methods have been developed based on the detection of specific antibody-antigen complexes. In order to detect whether an antibody has bound to its target antigen, several possibilities are available. The enzyme-linked immunoassay (enzyme-linked immuno absorbent Assay, ELISA) is one of these procedures. In principle, the ELISA can be used for the determination of any antigen, hapten or antibody; it's predominant application is in the area of clinical biochemistry. Hereby, it is used to measure, for example, hematological factors as well as the concentrations of serum proteins such as immunoglobulins, oncofetal proteins and hormones such as for example insulin. For the diagnosis of infectious diseases, microorganisms such as Candida albicans, rotaviruses, Herpes viruses, HIV or hepatitis B surface antigens are determined in this way. Moreover, immunochemical analysis methods are used for detection of antibodies for the purpose of diagnosing earlier or current infectious diseases (e.g. HIV, hepatitis).

An ELISA protocol typically comprises the following steps.

1. The sample supposed to contain a specific molecule or a certain organism is fixed to a solid support (e.g. microtiter plates made of plastic).

2. Antigens (protein, peptide, hapten-conjugate, etc.) are detected by specific binding of a specific antibody (primary antibody), which is directed against the respective antigen as described under 1. Hereby, the primary antibody can be labeled per se (e.g. radioactive) and can therefore be localized directly (e.g. by radioautography). Alternatively, the procedure can be continued according to the following paragraph.

3. Frequently, instead of this, a second antibody (secondary antibody) is added which binds specifically to the primary antibody but not to the antigen specified under 1. This second antibody is frequently coupled chemically with an enzyme (indicator system) which catalyzes the conversion of a colorless substrate into a colored product (e.g. alkaline phosphatase, horseradish peroxidase etc.). The second antibody is typically directed against the constant segment of the first antibody. Unbound secondary antibodies are removed by washing.

4. Addition of a colorless substrate which is converted into a colored product.

In the absence of any binding of the primary antibody to the antigens present in the sample, the primary antibody is removed in the first washing step. As a consequence, the enzyme-labeled second antibody also fails to bind, i.e. the a assay mixture remains colorless. If the respective antigenic structure is available, the primary antibody can bind and the second antibody can bind consecutively. The enzyme coupled to the second antibody catalyzes the color reaction whose product can be detected easily (e.g. photometrically). The observed enzyme activity is proportional to the content of specific antigen respectively antibody (from Glick, B. Pasternak, J. Molekulare Biotechnologie, Spektrum Akademischer Verlag, 1995, p. 201 ff.).

In order to perform binding assays, an indicator system (e.g. horseradish peroxidase) is required which permits the visualization of the immune reaction which has occurred. The visualization is based on the stable linkage between the analyzed reactant (antigen or antibody) and an indicator system. As indicators (amplifiers), fluorescent dyes, luminescent dyes, radioactivity, enzymes etc. are used. The indicators can be linked covalently or non-covalently to the respective reactant. For example, antigen-antibody binding, biotin-avidin binding or lectin binding can surve the purpose of stable non covalent linkage between indicator and the reaction partner to be detected.

In case of the direct method, the primary antibody is covalently linked to the indicator. The indirect setup circumvents the labeling of the primary antibody. The primary antibody is detected by an antibody which is labeled with an indicator. This secondary antibody which is obtained from a different animal species binds to all primary antibodies of any specificity from the first animal species.

Yet another method of detection consists in the method whereby three antibodies are used subsequently. The primary antibody from species A is detected by a non-labeled secondary antibody from species B which is present in excess. This is followed by the addition of the tertiary antibody from species A which is linked with an indicator. The secondary antibody (bridging antibody) serves as a bridge between primary and tertiary antibody. Through the use of several consecutive antibodies, the sensitivity can be enhanced.

Alternatively, the visualization of the bound primary antibody can occur via other binding systems. The avidin-biotin-complex-binding is an appropriate system (ABC system). Hereby, the primary or the secondary antibody must be present in biotinylated form. The indicators are likewise biotinylated and are bound to the tetravalent avidin under saturation of three binding sites. The fourth avidin binding site can bind the biotinylated primary or secondary antibody. Multiple biotinylation of the indicators used results in very large avidin-enzyme complexes which increase the sensitivity of the assay system (instead of avidin, streptavidine can be used). (from Bioanalytik, F. Lottspeich, H. Zorbas, Spektrum Akademischer Verlag, 1998, page 91 ff). With this procedure, there is a problem of a further enhancement of sensitivity.

Signal Amplification Through the Use of a Derivatized Multimeric Lumazine Synthase in Solution or on an arbitrarily selected surface:

1. By interpolation of a biotinylated multimeric lumazine synthase conjugate (linker protein) between primary antibody and indicator: A lumazine synthase containing up to 60 biotin molecules (e.g. bound through a short linker to the lumazine synthase in order to avoid steric hindrance) on its surface hereby adopts a special position due to its spherical, multimeric structure. The binding between antibody and linker protein respectively between linker protein and indicator occurs through the use of an avidin bridge or a streptavidine bridge. Alternatively, avidin- or streptavidine-labeled primary antibodies respectively indicators can be used. Linker proteins can be bound to 59 of the 60 biotin molecules on the surface of the multimeric linker protein, whereby only one biotin molecule is required for binding between primary antibody and linker protein. Through the resulting multiple binding of enzymes mediating the color reaction, an extreme signal amplification is obtained, where by the signal strength increases proportional to the antigen concentration.

2. Through the use of heterologomeric biotinylated lumazine synthase conjugates: Through the reassociation, according to the invention, of different lumazine synthase variants (for example combination of 1 to 3 antigen-containing lumazine synthase monomers with up to 59 biotinylated lumazine synthase monomers), a heterooligomeric lumazine synthase conjugate is generated which contains a reactant (e.g. antigen) as well as several biotin molecules. To the biotin molecules, streptavidine-mediated (or avidin-mediated or anti-biotin-antibody-mediated) indicator molecules are linked. In an exemplary fashion, two modes of use are described: A) A lumazine synthase conjugate comprising 1 to 5 short peptides of antigenically active viral or bacterial surface proteins (antigenic determinants) and up to 60 biotin molecules in covalent linkage serves as detection molecule for immobilized antibodies which stem from a patient's serum or other fluids. B) Characteristic antibodies against certain infectious diseases are harvested with the help of special immobilized epitopes (parts of surface antigens of the respective pathogenic organisms; antigenic determinants) from the respective body fluid. A lumazine synthase conjugate also containing about 1 to 5 copies of the epitopes designated above and up to 60 biotin molecules in covalent linkage serves as detection molecule for the antibodies bound to the immobilized epitope. In both cases (A and B), a color reaction is obtained through an arbitrarily selected, streptavidine-coupled enzyme which forms a complex with the biotinylated lumazine synthase. Through the interposition of this multiply biotinylated linker protein (lumazine synthase conjugate) and the multiple binding of color-mediating enzyme caused hereby, a signal amplification is achieved.

3. By application of heterooligomeric, non-biotinylated lumazine synthase conjugates: Through the reassociation of different lumazine synthase variants, according to the invention, a heterooligomeric lumazine synthase conjugate is generated which comprises a reactant (e.g. an antigen which can specifically bind antibodies from a patient's serum) in one copy as well as epitopes in multiple copies which are recognized by indicator-labeled antibodies. This again results in signal amplification through multiple binding of antibody-indicator-complexes to the multimeric protein.

Biosensors

Classical biochemical methods of analysis such as the immunoassay are based on chemical reaction systems in liquid state. A possible alternative consists in the application of solid-phase measuring devices or biosensors. During the last years, the use of biosensors as rapid and sensitive test systems for the detection of diverse materials and molecules is finding progressively more applications. A biosensor consists of at least three components: a biological receptor, a transducer and a coupled electronic system. In an immune sensor, the biological receptor can be an antibody or an antigen coupled to the transducer in a variety of ways. In both variations, the sensor enables the measurement of specifically formed antigen-antibody-complexes.

For the use as chemical sensors in liquids, e.g. in sera, volume vibrators are especially suitable. They include quartz vibrators laminated on a specially treated surface (according to the assay principle) with antigenic proteins or monoclonal antibodies. When alternating current is applied to the quartz, the crystal is excited to elastic vibrations whose amplitude reaches a maximum when the electrical frequency coincides with a mechanical Eigen-frequency of the respective quartz. These vibrations can be detected by appropriate measuring devices. When a quartz crystal laminated with antigens is placed in a solution containing specifically binding antibodies, the latter bind to the surface, thus modulating the mass of the sensor. The vibronic frequency is hereby modulated, thus indicating the binding of an antibody. Besides these piezoelectrical immunosensors, efforts are being made to develop measuring techniques whose mode of function is similar to potentiometric electrodes resembling those of pH-meters. In this case, it is attempted to monitor the modification of the potential which is generated upon formation of an antigen-antibody-complex on a thin equilibrated layer of silica gel on the surface of the pH-sensitive glass membrane. Yet another possibility for immunosensor measurements consists in the immobilization of proteins (antibodies or antigens) on the surface of an optical fiber. Interfering waves and surface plasmons are the optical phenomena which are most frequently used for this purpose. An interfering wave is formed when light propagating along an optical fiber is reflected internally. This interfering wave is the electromagnetic energy arising at the interface of optical fiber and liquid. The energy is absorbed when absorbing molecules are present at the interface, such that the degree of absorption is proportional to the amount of absorbing material at the interface. The formation of antigen-antibody complexes, whereby the antigen or the antibody is bound to the fiber surface, can be detected in this way. In the case of surface plasmon resonance, a metal-coated glass surface is used as optical device, whereby an internally totally reflected light beam generates an induced electromagnetic surface wave or plasmon. A detectable surface plasmon resonance arises at a specific angle of the incident light, which depends critically on the refractive index of the medium contacting the metal film. Thus, modifications of this layer, such as those that can be expected after the formation of antigen-antibody complexes, can be measured.

Potentiometric immune sensors comprise ion sensitive field effect transistors. A receptor (the antibody, antigen or other receptor) is hereby attached to the semiconductor gate of the transistor. The binding of an analyte to the receptor generates a modification of the charge distribution and thereby an activation of the field effect transistor (from Modrow S., Falke, D., Molekulare Virologie, Spektrum Akademischer Verlag, Heidelberg, Berlin, Oxford, p. 108; Lidell, E. Weeks, I. Antikorper-Techniken, Spektrum Akademischer Verlag, Heidelberg, Berlin, Oxford, pp. 154 ff.). An increase of sensitivity is also desirable in case of these sensor methods.

Signal amplification through utilization of derivatized, multimeric lumazine synthase molecules on the signal-mediating surface:

Artificial protein molecules on basis of lumazine synthase can serve as carrier protein, for the construction of a biosensor, e.g. for presentation of antigenically active catcher peptides for the detection of antibodies against certain infections. Through the formation of mixed lumazine synthase conjugates, according to the invention, the respective peptides can be incorporated into an icosahedral structure, together with a biotin molecule which mediates binding. In this way, up to 59 identical or different antigenically active peptides (e.g. domains of virus surface proteins) in connection with a biotin molecule, can be presented on top of an icosahedral molecule. Through the utilization of several different multimeric lumazine synthase conjugates, a representative peptide library can be placed on a single sensor. Binding of the multimeric lumazine synthase conjugate to the surface of a transducer can be enabled, for example, via streptavidine-biotin coupling.

The sensitivity of such an assay system is significantly enhanced by displaying several antigenic determinants, since not only one single antibody but several antibodies directed against a specific pathogen can be detected. Moreover, no well-founded detailed knowledge on specific protein segments contributing to the binding of antibodies is required, since several proteins of the respective pathogen can be presented on the sensor with limited effort. Since streptavidine/biotin-coupling can be used for all epitope presentations, in order to build up a sensor, the same surfaces coated with avidin or streptavidine are required throughout, i.e. the experimental setup does not have to be modified. The respective individual epitope-presenting or biotinylated lumazine synthase subunits can be easily prepared by recombinant technology. This has significant advantages for the development respectively evaluation of diagnostic procedures of this type.

Through the presence of up to 59 catcher peptides on one molecule, the surface of the sensor chip (e.g. field effect transistor, plasmon resonance transducer surface etc.) can be increased extremely, thus providing an enormous enhancement of sensitivity. Problems of stability and specificity are not to be expected upon utilization of a thermostable carrier protein and the biotin/streptavidine system.

In the same way, small molecules can be bound to the surface by simple chemical coupling. As coupling sites for this purpose, singular exposed reactive amino acids are available on the surface of the spherical protein.

Principal Structure of a Layer System on Basis of Multimeric Lumazine Synthase:

A functionalized lumazine synthase with 60 identically or differently modified subunits is linked to a surface (e.g. transducer surface or other arbitrarily selected surface located on a transducer) via an anchor (peptide, fatty acid etc.). The detection sensitivity for binding of foreign molecules on the surface of the lumazine synthase is hereby enhanced through a high number of functional groups (e.g. epitopes for antibody detection, antibodies for detection of foreign molecules in solution or other receptors).

Preparation of Vaccines (In Vitro)

Vaccinations are conducive to an immunological resistance against infectious agents. Vaccines serve predominantly for prevention, i.e., they should result in the buildup of a protective potential in the immunized persons whereby it will protect them, upon contact with the respective infectious agent, and thereby protect them from disease. The injected orally applied vaccine is conducive to the formation of antibodies and/or a cellular immune response in the organism. Consequently, upon future exposure, the infectious organism is killed or neutralized with the result that the disease does not break out.

Infections with bacteria, viruses, fungi and protozoa are a main factor of morbidity and mortality worldwide. Through the increasing development of resistance against virtually all available antibiotics, a deterioration of the morbidity situation is also expected in industrialized countries. The development of novel vaccines is therefore of the highest medical significance.

As vaccines, e.g. attenuated viruses can be applied. Attenuated viruses resemble infectious agents causing disease, albeit they differ from them with regard to the virulence behavior; thus they cause only a limited respectively attenuated infection, thereby inducing the formation of neutralizing antibodies and cytotoxic T-cells. Mutations in the genome of wild type viruses form the molecular basis of attenuation. Attenuated viruses typically generate a very good immune protection which remains intact for several years, but they carry the risk of backmutation to the wild type form in the course of the attenuated infection.

Yet another possibility for immunization of humans and animals consists in the presentation of antigenically effective parts of surface proteins on top of other, non-pathogenic viruses, e.g. plant viruses. The gene fragments specifying an antigenic determinant (e.g. surface protein) of the pathogenically active virus are integrated into the genome of the non-pathogenic virus (Dalsgaard et al., 1997). The foreign protein is thereby presented on the surface of the non-pathogenic virus. It is however not possible to integrate DNA fragments above a certain limiting size into the viral genome. Hence, it is necessary to know exactly which proteins of the infectious virus are relevant for the generation of a protective immune response. A vaccine of this type cannot generate an immune response with the same diversity as that arising in the course of an infection with the wild type virus or its attenuated variant. With this type of recombinant vaccine viruses, the immune response is limited to a selected protein.

Vaccines consisting of synthetic peptides with a length of 15 to 30 amino acids represent a vaccine form which is presently under investigation. In this case, individual epitopes of viral proteins which cause the development of neutralizing antibodies are selected and synthesized chemically. Solid and detailed knowledge on protein segments causing a virus-neutralizing immune response is also required in this case. On basis of the high genetic variability of most viruses and the different capacity of individuals to recognize specific protein regions immunologically, it would be necessary to combine several epitopes in a vaccine based on synthetic peptides. Since there is, beside aluminum hydroxide, no other suitable adjuvant that is generally suited for humans in order to in enhance the immune response sufficiently, no vaccine based on synthetic peptides is hitherto available (from Modrow S., Falke D., Molekulare Virologie, Spektrum Akademischer Verlag, Heidelberg, Berlin, Oxford, p. 87 ff). In contrast to short peptides, high molecular weight molecules such as proteins and carrier-fixed proteins are very well suited as vaccines because they can be applied without the use of auxiliary materials and all the same afford a very good immunity. The redundant occurrence of antigenic determinants in high number, such as in case of viruses or bacteria, on immunogenic molecules of high molecular weight is favorable for the desired high antigenicity, i.e. a preventive immune response. Lumazine synthase is particularly suited for this purpose because of its icosahedral structure. The lumazine synthase consists of at least 60 subunit, i.e. at least 60 equivalent or different antigenic determinants can be presented on one molecule. The lumazine synthase has a high molecular weight structure and a surface structure which is similar to that of certain viruses, i.e. a high antigenicity can be expected. Vaccines of this type are free of viral genes and can be prepared with little effort in high yield. Since large viral proteins can be presented, detailed and well-funded knowledge on protein segments causing a virus neutralizing immune response is not required.

The proteins generated by genetic engineering which are the subject of the present invention are based on the covalent linkage of a wild type lumazine synthase or a modified lumazine synthase with partial structures of viruses, bacteria, fungi, protozoa or toxins. The linkage can occur at the N-terminus and/or at the C-terminus of the lumazine synthase. Additionally, the peptides to be presented can be inserted at appropriate sites into the sequence in such a way that they are presented in the form of a loop on the surface of the multisubunit protein. It is thereby possible to present a given immunological determinant in a welldefined high number, e.g. 60-fold or 120-fold according to the invention, on top of an icosahedral molecule consisting of 60 subunits with a triangulation number T=1. Moreover, it is also possible to prepare associates of high molecular weights comprising more than 100 subunits (triangulation number T=2 or higher) which are thereby able to present an even larger number of epitopes.

The association, according to the invention, of subunits with different peptide or protein sequences spliced by genetic engineering also offers the possibility for the production of protein molecules which present different antigenic sequences on one given molecule.

DNA Vaccine

Since the beginning of the 90's, the possibility to use DNA as vaccine has been under study. The nucleic acids used contain genes or parts of genes of a pathogenic organism specifying an immunogenic protein. For the development of these vaccines, detailed knowledge on the immunologically important components is most useful. The genes used predominantly specify surface components of a pathogen or parts of bacterial toxins. They are integrated, together with regulatory elements for the control of their expression, into a vector system which is applied in the form of pure DNA by injection into muscle tissue where it is expressed. Especially in muscle cells, DNA can be detected over long periods as epsisome, since obviously it is degraded only very slowly. When these respective genes are expressed, the organism can generate a humoral as well as a cellular immune response. Up to now, this form of vaccine has been studied in animal models.

Gene constructs which specify fusion proteins consisting of protein components of pathogenic microorganisms and of lumazine synthase are in principle suitable as DNA vaccines. A DNA vaccine consisting of a gene coding for a lumazine synthase (particle-forming component) and a selected gene of the pathogenic agent can be expressed intracellularly, thus affording the production of antigen that can stimulate the immune system over long periods. According to current experience with lumazine synthases from different organisms, the assembly of the icosahedral molecules in vivo should be possible without auxiliary molecules (cf. chaperonins).

Oral Vaccines on Plant Basis

If the immunologically active protein component of the infections agent responsible for a protective immune response is known, the gene specifying that peptide can be incorporated into a eukaryotic expression vector. Subsequent to transformation of plant cells with this DNA, transgenic plants can be obtained which express the respective gene. The selected protein component can be incorporated by consumption of the plant and can thereupon generate an immune response.

As a particle forming protein, lumazine synthase to which parts of the immunologically active protein of the pathogenic have been fused is particularly suitable. By the use of a thermostable, particle-forming lumazine synthase (e.g. from Aquifex aeolicus) as carrier protein, even boiling-resistant vaccines can be generated.

Multifunctionally Derivatized Immune Therapeutics on Basis of the Multimeric Lumazine Synthase

Vaccines are intended to inhibit the multiplication of a pathogenic agent and thereby prevent infection. In certain cases it is difficult to develop a reliable vaccine since the pathogenic organism is not accessible to antibodies or, as in the case of acquired immune deficiency (AIDS), too little is known about the pathogenic agent (HIV). The targets of HIV are helper T-cells (helper cells) of the immune system, whereby the most important functions of these cells are impaired. When HIV penetrates into helper cells, the virus is protected from the immunological attack. In the subsequent course of the disease, the infected cell can be destroyed by the production and liberation of HIV particles. An infected cell can thereby become a “factory” for the production of additional virus particles. The most important consequence of HIV infection is the fact that the immune system can no more provide protection of ordinary infectious disease. The first step in HIV infection is the interaction of a 120 kDalton glycoprotein (gp 120) of the viral capsid with the CD4 receptor at the surface of the helper cells.

Antibodies against CD4 block the infection of helper cells under in vitro conditions. The rate of infection is also reduced by an excess of free CD4 protein. A fusion protein comprising parts of the CD4 protein and the F_{C} component of an immunoglobulin was developed in an attempt to protect the helper cells as well as to eliminate the virus. The fusion protein is designated CD4 immunoadhesin. The molecule binds gp120 and blocks HIV; both said activities depend upon the CD4 component. The capacity of the fusion protein to bind to cells with F_{C} receptors and the long half life in plasma are due to the immunoglobulin component. After binding of the immunoadhesin to the free virus or to an HIV-infected cell, an antibody-dependent, cell-mediated cytotoxic reaction conducive to the destruction of the virus or the HIV-infected cells is initiated (from Glick, B., Pasternak, J., Molekulare Biotechnologie, Spektrum Adademischer Verlag, 1995, p. 245).

The efficiency of that strategy may be improved by the use of a multimeric derivatized lumazine synthase. It is also possible to use a functionalized lumazine synthase comprising CD4 protein components as well as F_{C} components. The efficiency should increase considerably since many of these units rather than one single functional unit are present in the molecules.

Instead of the CD4 component, antibodies (e.g. specially developed single chain antibodies) directed against a tumor marker (e.g. teratocarcinoma antigen) may be introduced into the multimeric protein, and the functionalized fusion protein may be used for the therapy of cancer.

An additional mode of application could consist in the combination of an antibody against a tumor marker with metallothionein. The multimeric lumazine synthase is hereby decorated with an antitumor antibody and up to 59 metallothionein molecules. The metallothionein molecule, in turn, are loaded with radioactive elements (characterized by short half life time) which are suitable for radiation therapy (e.g. technetium 59). In the course of the therapy, the protein complex binds to the tumor via it's antibody component, thereby closely apposing the source of radiation to the tumor tissue. Similar constructs can also be used for diagnostic purposes, e.g. radioactive detection of malignant tumors.

Utilization of Lumazine Synthase Conjugates for the Characterization and Purification of Antibodies

The basis of the foreign peptides is provided by DNA sequences specifying a specific epitope. The sequence of the additional peptide segment can be determined exactly by selection of the DNA sequence. However, it is also possible to incorporate peptide sequences characterized by a stochastic amino acid sequence over their entire length or in partial segments. Multiple stochastic variability can be achieved by the use of synthetic oligonucleotides comprising randomly generated sequence segments in order to form representative peptide libraries. These randomly generated peptides are presented on the surface of the lumazine synthase and are thus accessible for antibody binding.

The resulting lumazine synthase variants (with stochastic variability of the foreign peptides) can be used, for example, for the characterization of antibody binding site. By isolation of antigen-antibody complexes with subsequent sequencing of the bound peptide segment (N-terminal Edman sequencing or sequence determination by mass spectrometry), the selectivity of the binding site of an antibody can be characterized.

It is also possible to search specifically for antibodies characterized by a specific antigen recognition (whereby the antigen sequence is known in this case). By application of mixed conjugates, i.e. lumazine synthase conjugates comprising a desired foreign peptide (in multiple form) as well as a biotinylated component (in single form), antibodies can be selectively purified from mixed population. The use of streptavidine or avidin coupled to a solid phase is appropriate for the purpose. The purification, according to the invention, can also be performed on basis of other affinity materials. The antibodies can be eluted by known standard procedures.

Solutions for the Described Technical Problems

The solution of the described technical problems is achieved by providing the application forms characterized by the patent claims. The objective of this invention is the use of lumazine synthase molecules as carrier proteins for foreign proteins, peptides and/or other molecules from the area of organic chemistry. Moreover, the objective of this invention is a method for the selective, recombinant incorporation of said foreign proteins respectively peptides into loops or, according to the invention, preferentially at the N-terminus and/or at that C-terminus of lumazine synthases. The method involves an in vivo association of different lumazine synthase conjugates by way of co-expression of the respective genes in one given cell. Moreover, the method includes the possibility of in vitro reassociation of individually designed lumazine synthase conjugates by formation of spherical particles by way of denaturation/renaturation of monomeric subunits which can be carried out with or without the use of a ligand which supports the folding.

The technology provides lumazine synthase conjugates characterized by a peptide accessible to biotinylation (Tucker and Grisshammer, 1996; Schatz, 1993; Cronan, 1990) at the C-terminus. Moreover, the technology provides an artificial lumazine synthase molecule characterized by a well accessible basic amino acid (lysine) at the C-terminus. Moreover, the technology provides a lumazine synthase molecule characterized by a well accessible cystein molecule at the C-terminus. Both variants are suitable for chemical coupling of organic molecules. Coupling can be achieved by the generation of an amide bond or a disulfide bond between protein and coupling component. Chemical coupling according to the amide principle can also occur at the lysin residues which are naturally present on the surface of lumazine synthase molecules.

Moreover, the technology provides a thermostable, icosahedral lumazine synthase (from Aquifex aeolicus) which is suitable as carrier protein for the preparation of particularly stable lumazine synthase conjugates.

The procedure for the preparation of lumazine synthase conjugates involves the following steps:

I. Preparation of Fusion Vectors

A) Preparation of a DNA containing a gene for a lumazine synthase (e.g. by isolation from an organism, by PCR amplification with naturally occurring RNA or DNA as template or by DNA synthesis).

B) Introduction of suitable restriction sites for the later insertion of foreign DNA into the lumazine synthase gene; adaptation of the lumazine synthase sequence to particular requirements using known mutagenesis methods based on molecular biological and biochemical methods; insertion of the DNA into a cloning vector by application of known molecular biology methodology. (Alternatively, the DNA coding for the foreign peptide can be fused directly with the lumazine synthase gene using the polymerase chain reaction and synthetic oligonucleotides, whereby II.D must be granted.

C) Transformation of host cells with the resulting plasmid

D) Selection of transformants by use of antibiotics or other selection procedures

E) Analysis of transformants by means of molecular biology and biochemistry methods such as restriction mapping, sequencing, measurement of enzyme activity etc.

II. Insertion of a DNA Specifying a Foreign Peptide

A) Cloning of the foreign DNA by means of molecular biology methodology or preparation of a DNA by use of chemical synthesis methodology

B) Analysis of the DNA using molecular biology technology

C) Preparation of the DNA specifying the foreign peptide designated for fusion

D) Insertion of the prepared DNA at the 5′ and/or the 3′ end and/or into a loop region of the lumazine synthase gene in the vector prepared under I. in order to fuse the foreign gene with the lumazine synthase gene. The cloning must occur in such a way that all used gene segments are incorporated in the correct reading frame in order to arrange for all fused gene segments to be jointly translated into a fusion protein.

E) Transformation of host cells with the resulting plasmid

F) Selection of transformants using antibiotics or other selection procedures

G) Analysis of transformants by means of molecular biology or biochemistry methodology such as restriction mapping, sequencing, measuring of enzymatic activity etc.

III. Expression and Purification of the Hybrid Polypeptides

A) Fermentation of the host strain with the artificial fusion DNA using known microbiological methods

B) Expression of the fused artificial DNA in the transformed host cells as chimeric protein. The expression of the artificial DNA can involve a purposeful post-translational modification of the chimeric protein in vivo, e.g. phosphorylation, glycosidation, biotinylation etc.

C) Preparation of a cell extract with the fusion polypeptide

D) Purification of the fusion protein by means of chromatographic or other methods

E) If required: Solubilization and in vitro folding (renaturation)

F) If required: Chemical modification of the surface of lumazine synthase variants

G) If required: In vitro association under combination of different lumazine synthase variants

Additional Explanation of the Methodology:

The application of the present invention can involve a multitude of different vectors. Extra-chromosomal (episomal) vectors (e.g. plasmids), integration vectors (e.g. lambda vectors), Agrobacterium tumefaciens-based vectors designed for plants (e.g. Ti-plasmid). According to the invention, plasmid vectors are preferred. The plasmids used can have been isolated from natural sources or can be prepared synthetically. The selected plasmid should be compatible with the respective host strain. Therefore, it should have a replication origin suitable for the respective host strain. Moreover, the capacity of the vector should be sufficient for the used lumazine synthase variant as well as the fused foreign peptide. Moreover, singular restriction sites for the cloning of DNA fragments are required. The plasmid vector should have suitable features such as a resistance gene in order to enable appropriate selection procedures. The selection is necessary in order to distinguish host cells with and without plasmid.

If Escherichia coli is selected as host strain, vectors using a promoters sequences from bacteria phage T5 or T 7, an operator sequence, preferably the operator sequence of the Escherichia coli lactose operon (lacO), a cloning site with several singular restriction sites for restriction endonucleases and an efficient terminator sequence are preferred according to the invention. Moreover, the vector should have a replication origin providing for a high copy number of the extrachromosomal DNA in the host cells.

Prokaryotic expression systems are in general well-suited for the recombinant production of protein conjugates according to the invention. In certain cases, however, post-translational modifications may be required which cannot be introduced in prokaryotic organisms. For example, eukaryotic proteins cannot be glycosidated or phosphorylated. Therefore, eukaryotic foreign proteins (fused to lumazine synthase) requiring such a post-translational modification are expressed preferentially under the control of a strong promoter (e.g. AOX1) in lower eukaryots (e.g. Pichia pastoris) or under the control of a promoter specific for mammalian cells (e.g. rat preproinsulin promoter) in mammalian cells (e.g. COS7 monkey kidney cells) or under the control of a promotor (e.g. polyhedrin promoter) specific for insect cells (Baculovirus, Autographa californica). The respective factors used should be compatible to the said host strains.

For production of oral vaccines on basis of plants, it is for example possible to use vector systems on basis of the Ti plasmid of Agrobacterium tumefaciens. As an alternative to gene transfer in plants (e.g. monocotyl plant such as rice, wheat, maize etc.), physical methods (e.g. the gene gun technology, biolistic technology) can be used.

Naturally occurring proteins as well as proteins which do not occur in nature can be fused to the carrier protein (lumazine synthase). As sources of DNA, it is for example possible to use viruses, prokaryotic (eubacteria, archaea) and eukaryotic organisms (plants, animals). The DNA selected for fusion can also be prepared synthetically using established technology. Moreover, DNA can be prepared on basis of mRNA using reverse transcriptase.

The plasmid vectors obtained by recombinant technology are used for the transformation of host cells. Well characterized bacterial cells are preferred according to the invention. The host cells can also be eukaryotic cells. The host strains used should provide the enzyme systems required for expression of the fused polypeptide. Transformation techniques are well known in the field. Specific procedures are described in Maniatis et al. (1982). Subsequent to the transformation, transformants are analyzed. The plasmids are isolated and characterized by molecular biology methods such as restriction analysis and DNA sequencing.

The expression of the cloned DNA sequence in a prokaryotic or eukaryotic host cell can be performed by well-known technology. Cultivation of transformed host cells, according to the invention, for the preparation of recombinant fusion proteins proceeds under conditions which are favorable for the expression of the DNA sequence. Cell disruption subsequent to gene expression can be performed by all methods generally accepted for that purpose. Disrupted cells are separated into a soluble and an insoluble fraction by known separation procedures.

If the fusion protein is present in the insoluble fraction in the form of inclusion bodies, the pellet obtained by centrifugation is washed and subsequently dissolved by the addition of a solubilizer. Solubilization is preferentially performed in presence of reducing agents. Insoluble components are removed by known procedures. According to the invention, the renaturation step can be performed in presence of a stabilizing agent (5-nitro-6-ribitylamino-2,4(1H,3H)-pyrimidinedione).

Purification of the fusion proteins can be performed using known chromatographic or other biochemical methods.

Covalent coupling of molecules by chemical methods is enabled or facilitated by the introduction of a reactive amino acid using recombinant technology, preferably a lysine and/or cystein residue according to the invention, which is coupled to a flexible peptide linker. According to the invention, coupling can be performed by several different methods. The following examples are given specifically: a) Bismid esters are well soluble in water and can be coupled with the ε amino group of a lysine residue under mild reaction conditions (pH 7.0-pH 10.0). The resulting amide bond is stable. Lumazine synthases activated in this way can be used for coupling with other peptides. b) Carbodiimides belong to a group of compounds described by the general formula R—N═C═N—R′. The residues R respectively R′ can be aliphatic or aromatic moieties. Carbodiimides react preferentially with the ε amino group of lysine. c) m-Maleimido-benzoyl-N-hydroxysuccinimide ester (MBS) is a well studied heterobifunctional reactant. In neutral aqueous solution, MBS reacts initially via an acetylation type reaction under formation of an activated N-hydroxysuccinimide ester. A second peptide can then be bound via addition of a thiol residue to the double bond of the ester. d) N-Succinmidyl-3-(2-pyridyldithio)-propionate (SPDP) is a heterobifunctional reagent which can react under mild conditions with amino groups of the target proteins. The 2-pyridyldisulfide structure can then react with aliphatic thiols or a cystein residue of an additional peptide by thiol disulfide exchange reaction. The coupling reaction can proceed in the pH range of 5-9 and the reaction progress can be monitored photometrically. No reactions with other functional groups are known.

The preparation, according to the invention, of lumazine synthase conjugates by in vitro reassociation proceeds via a dissociation step and a subsequent folding/reassociation step. The dissociation can occur by a treatment with denaturating agents, e.g. urea or guanidine chloride, by modification of the pH value, by heat treatment or by other procedures. The monomeric chimeric proteins which are present after denaturation comprise a constant region of a lumazine synthase (respectively a modified lumazine synthase) and a variable region (a fused peptide which can be selected arbitrarily). Subsequently, the monomeric subunits can be mixed arbitrarily. Since each respective recombinant subunit comprises a respective constant lumazine synthase part, renaturation of the lumazine synthase core structure under formation of the natural icosahedral structure is possible. The renaturation can proceed in presence of a stabilizing agent (preferentially 5-nitro-6-ribitylamino-2,4(1H,3H)-pyrimidinedione).

The in vivo combination of different lumazine synthase variants proceeds by way of co-expression of the respective gene coding for the respective fusion polypeptide. Here by, the respective genes can be located on the chromosomal DNA of the host strain and/or on one or several plasmid vectors. By modulation of the expression of the lumazine synthase variants to be associated in vivo, a specific ratio of combinatorial variants can be established.

### LEGENDS TO FIGURES

FIG. 1 gives a schematic representation of an ELISA protocol for the determination of a specific antigen or a specific antibody. The antigen is bound to the microtiter plate. The enzyme (E) is coupled to the secondary antibody. The colorless substrate is converted to a colored product by the enzyme (E).

FIG. 2 describes the detection of an antigen by way of a biotin-labeled primary antibody. A lumazine synthase conjugate (amplifying linker molecule) comprising up to 60 covalently bound biotin molecules is linked to a biotinylated primary antibody via a streptavidine or avidin bridge (SA). The color reaction occurs by way of an arbitrarily selected streptavidine coupled enzyme (E) which forms a complex with the biotinylated lumazine synthase. Through the interposition of a 60-fold biotinylated linker protein (lumazine synthase conjugate) and the multiple binding mediated thereby of a color reaction mediating enzyme, an extreme signal enhancement is obtained, whereby the signal strength is proportional to the antigen concentration.

FIG. 3 describes the use of a lumazine synthase mixed conjugate for the diagnosis of infectious disease.

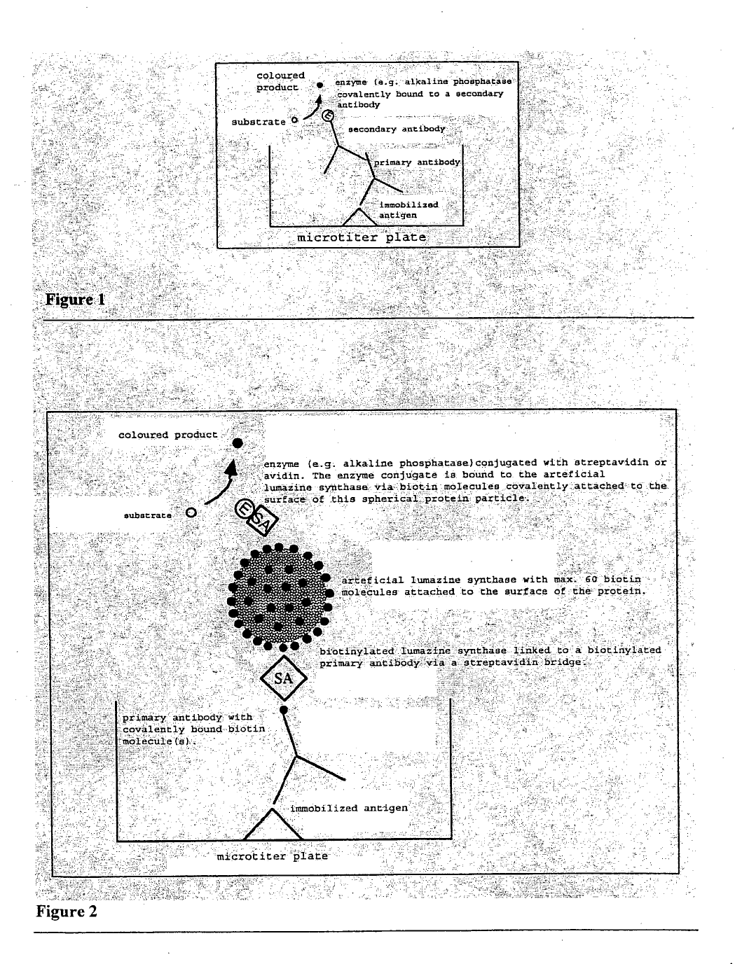

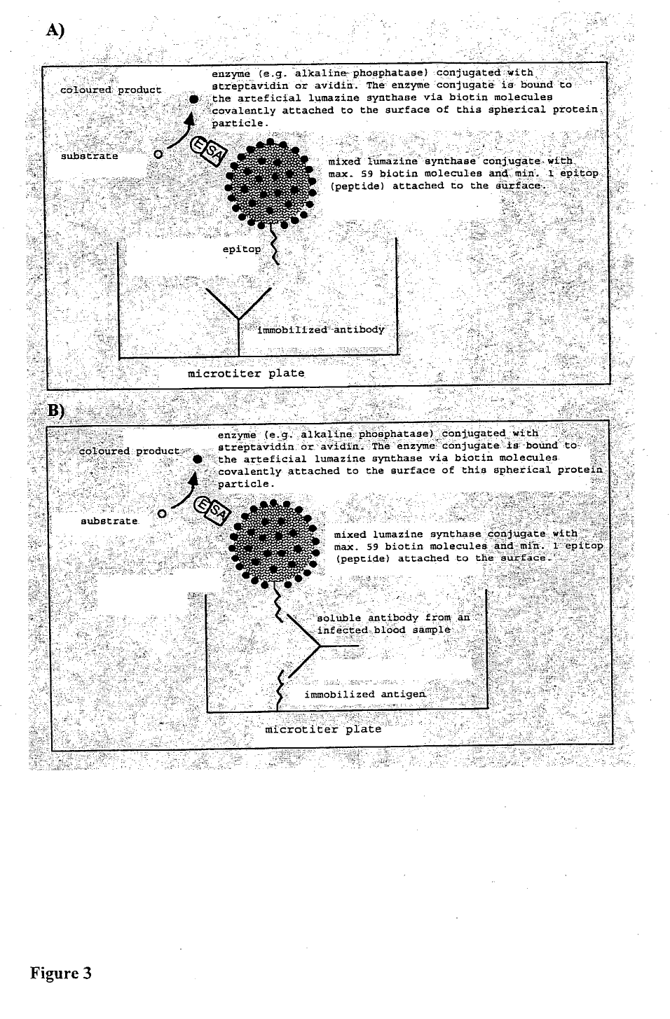

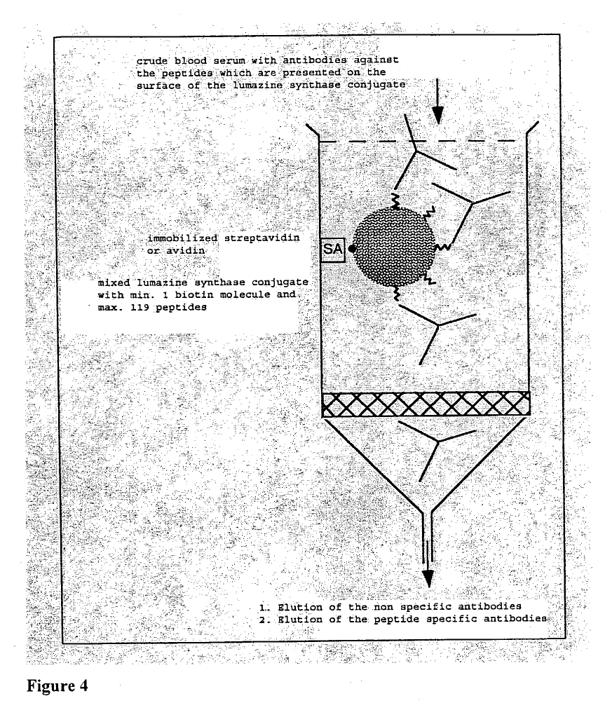

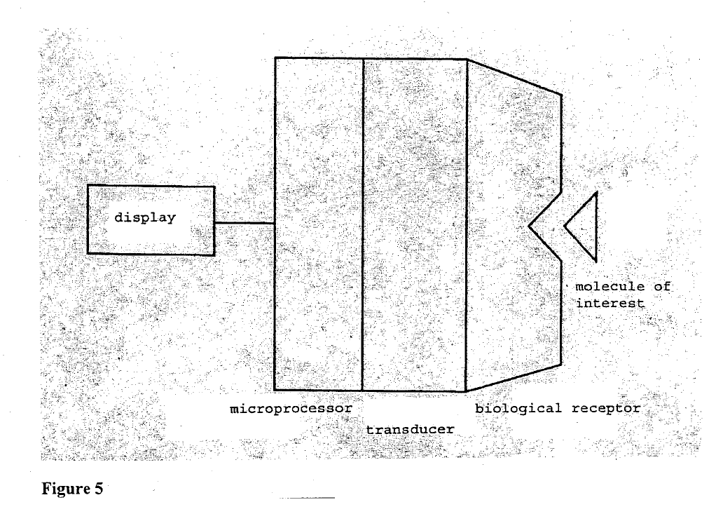

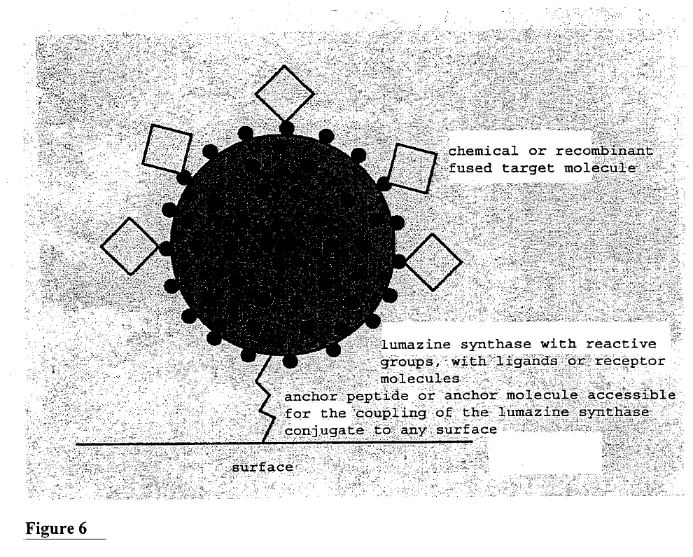

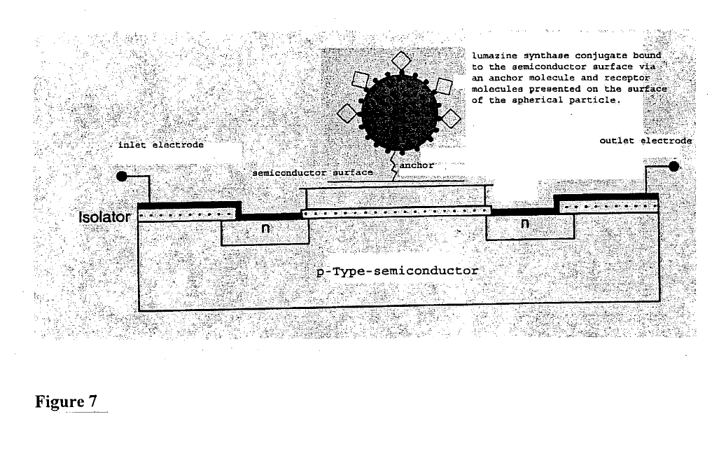

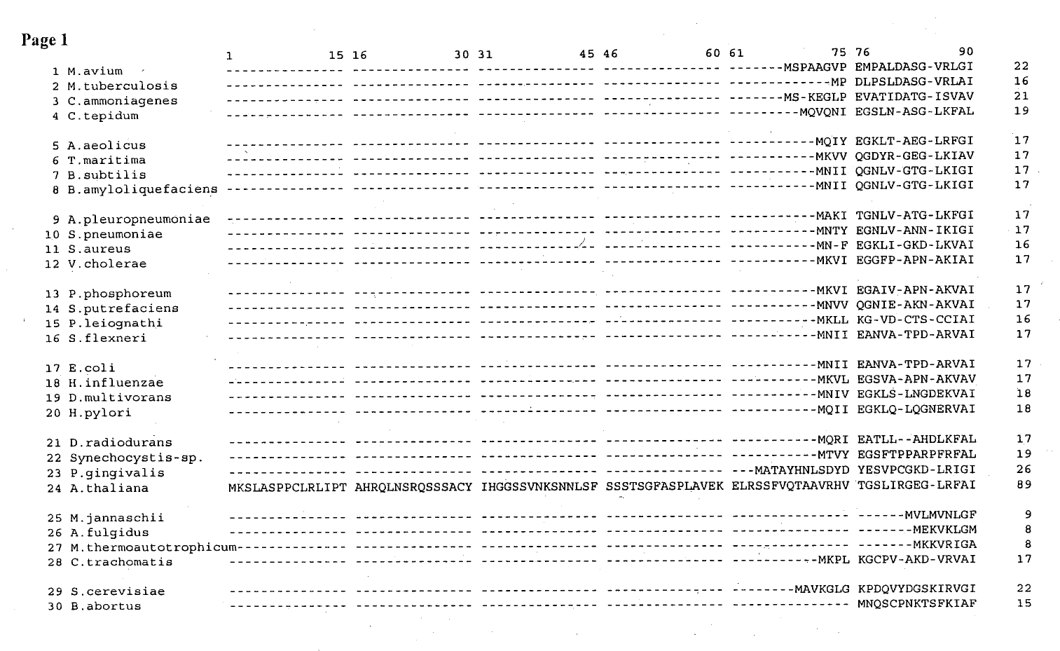

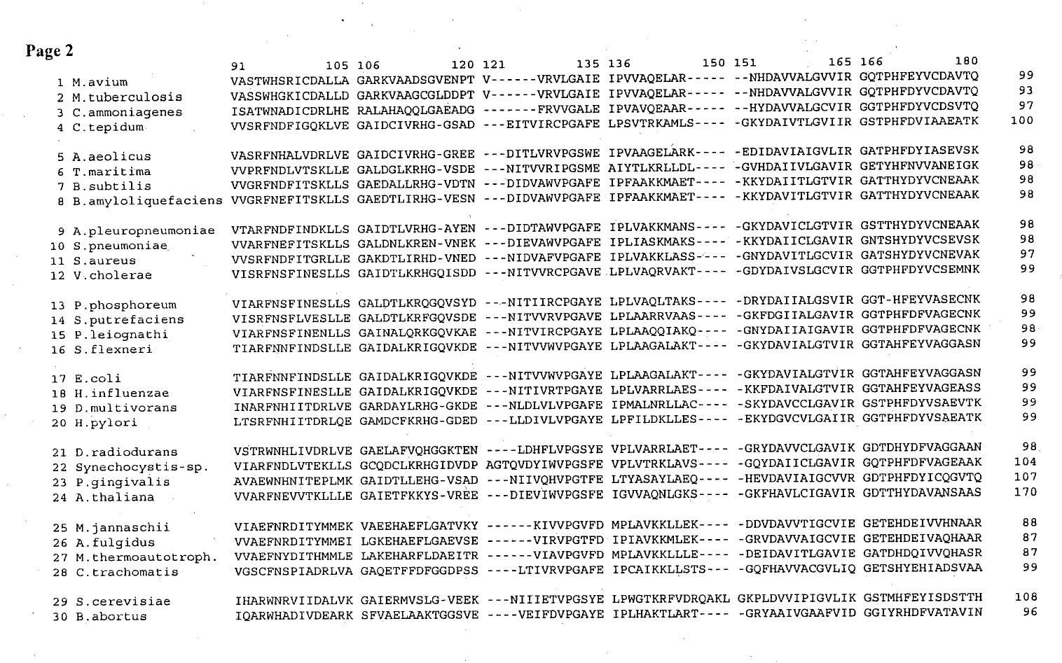

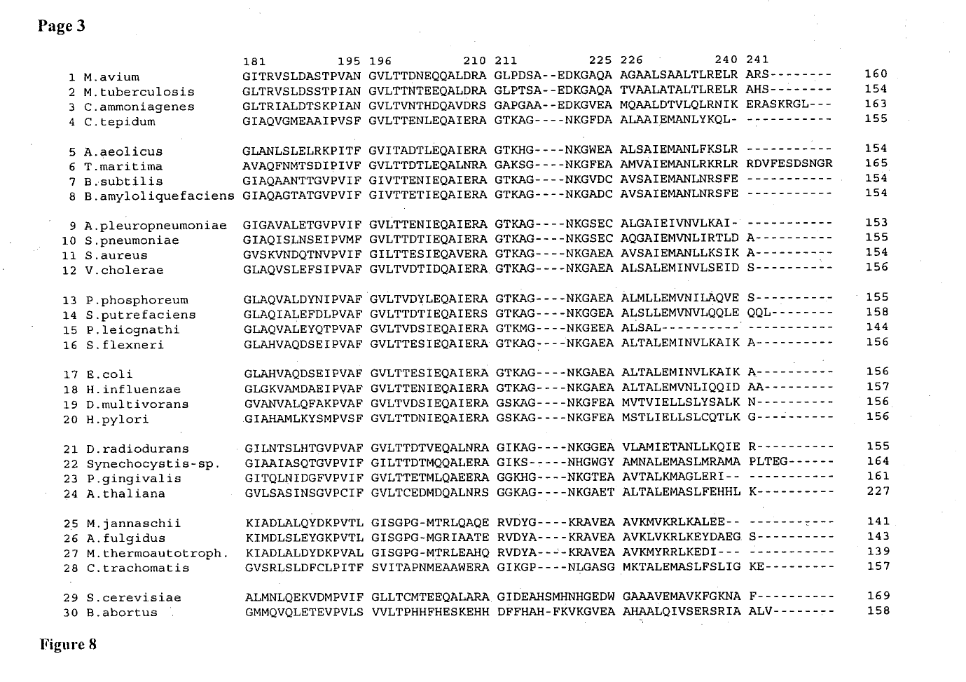

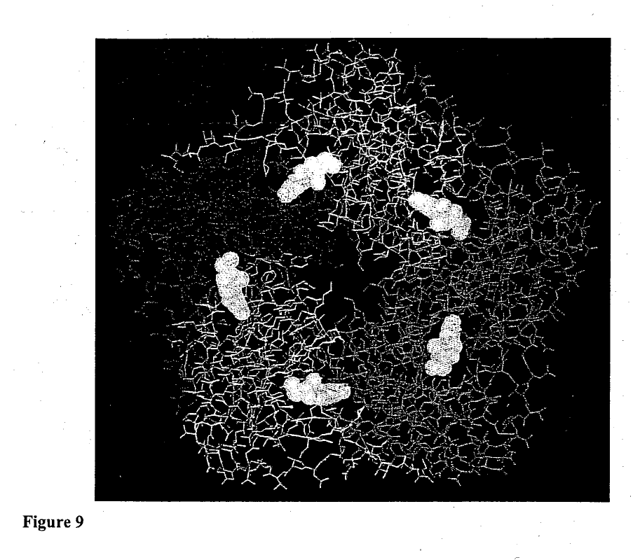

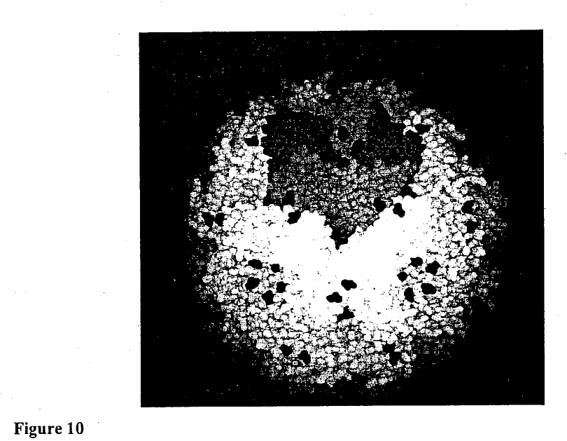

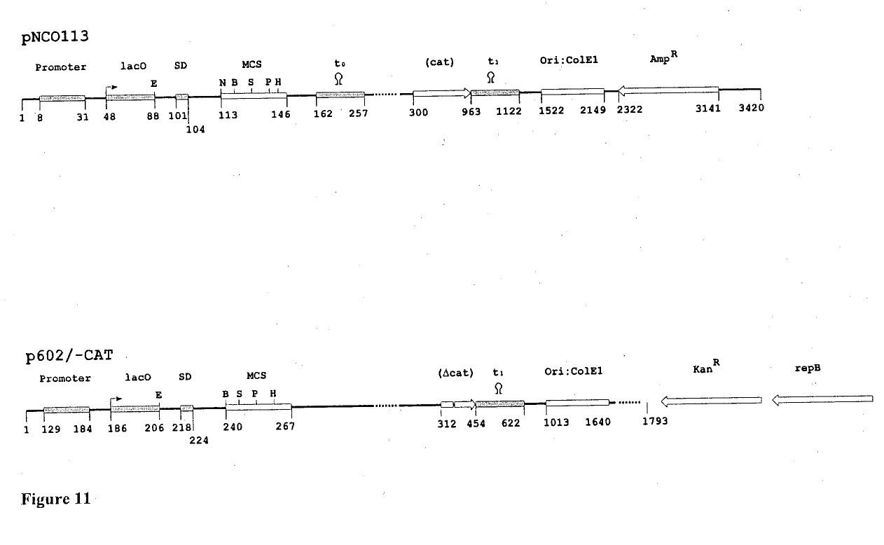

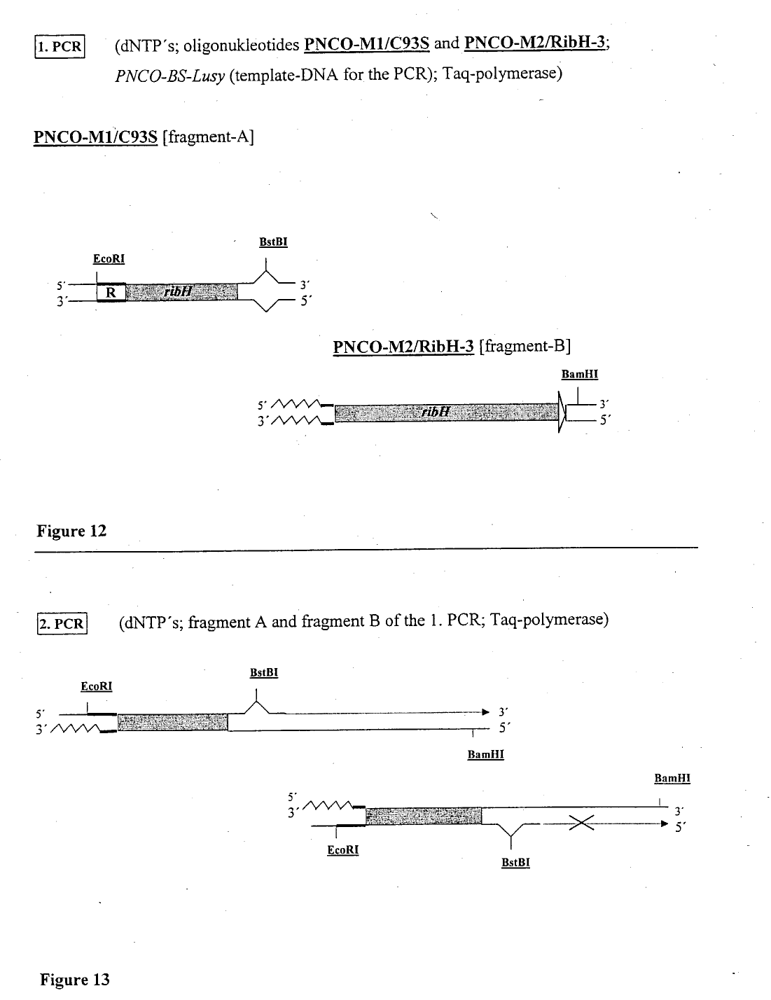

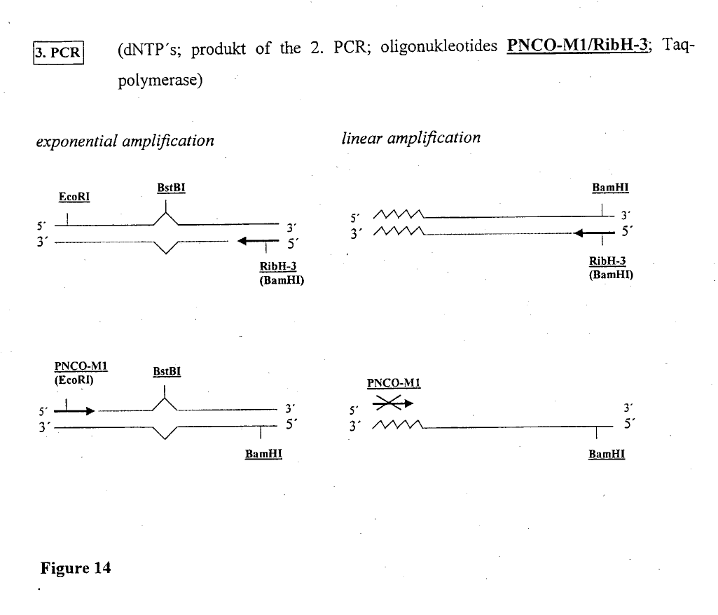

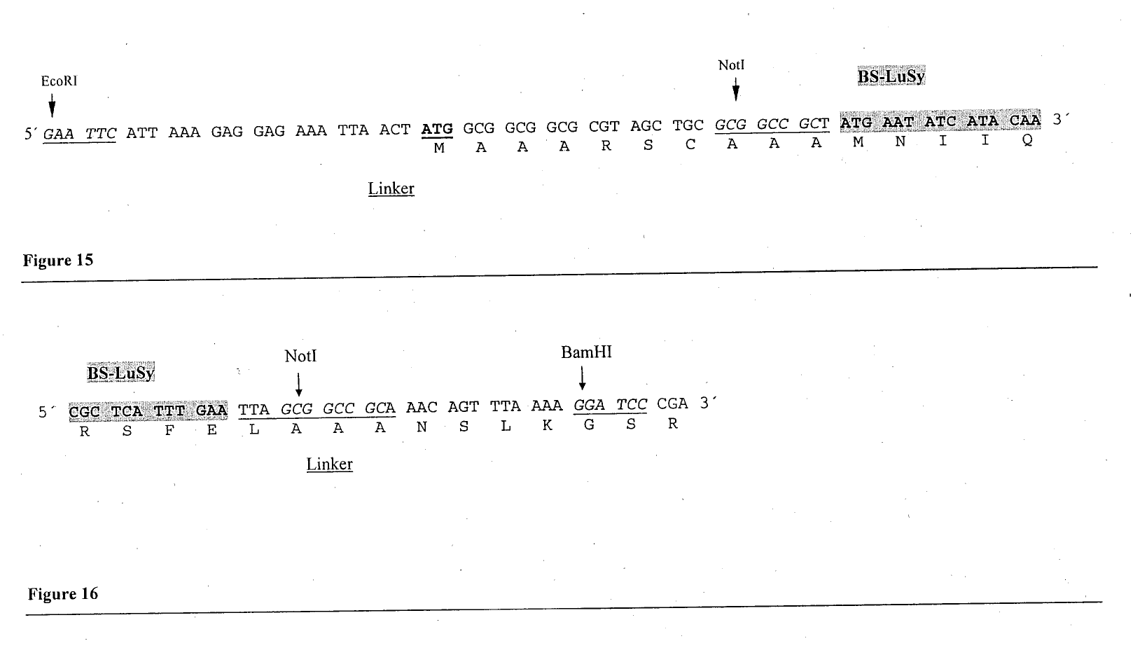

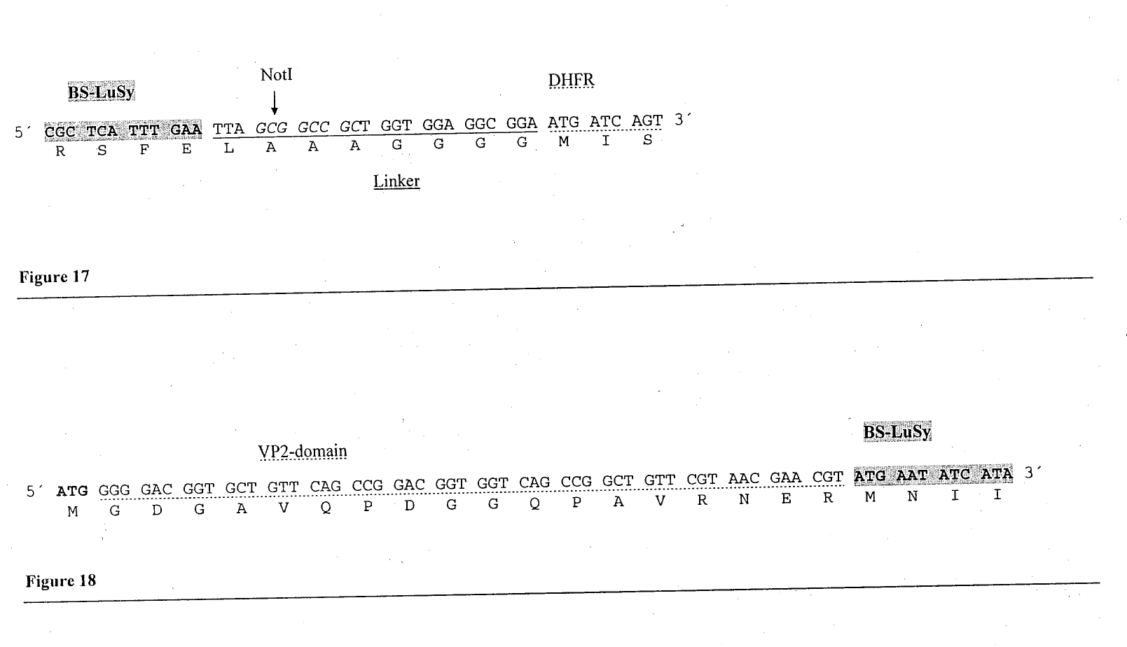

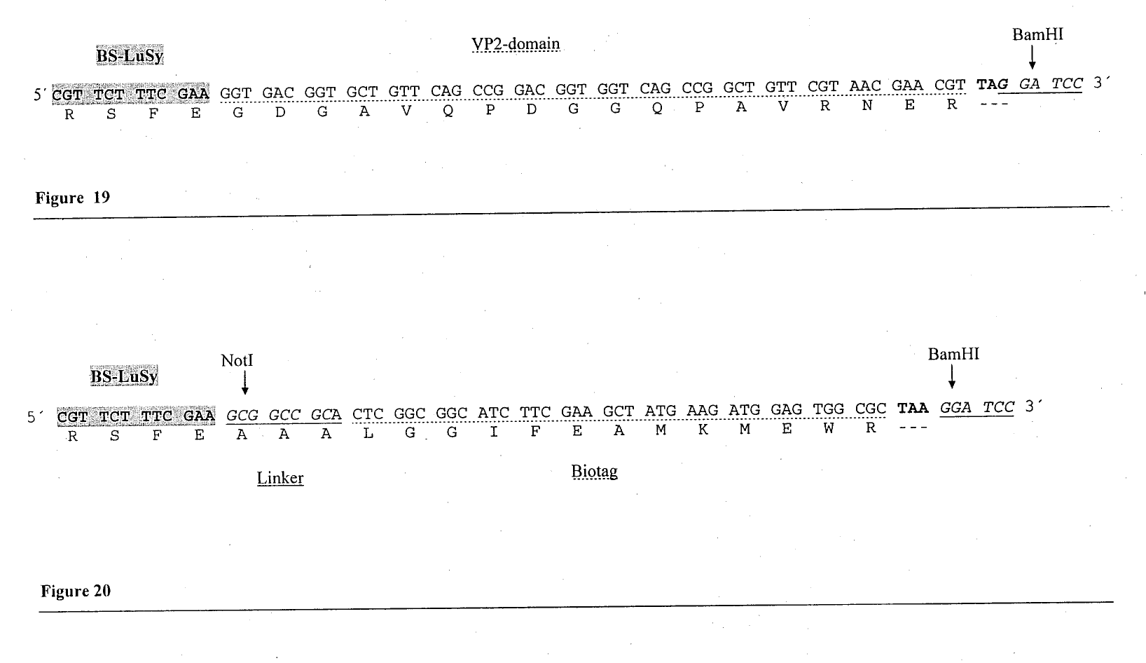

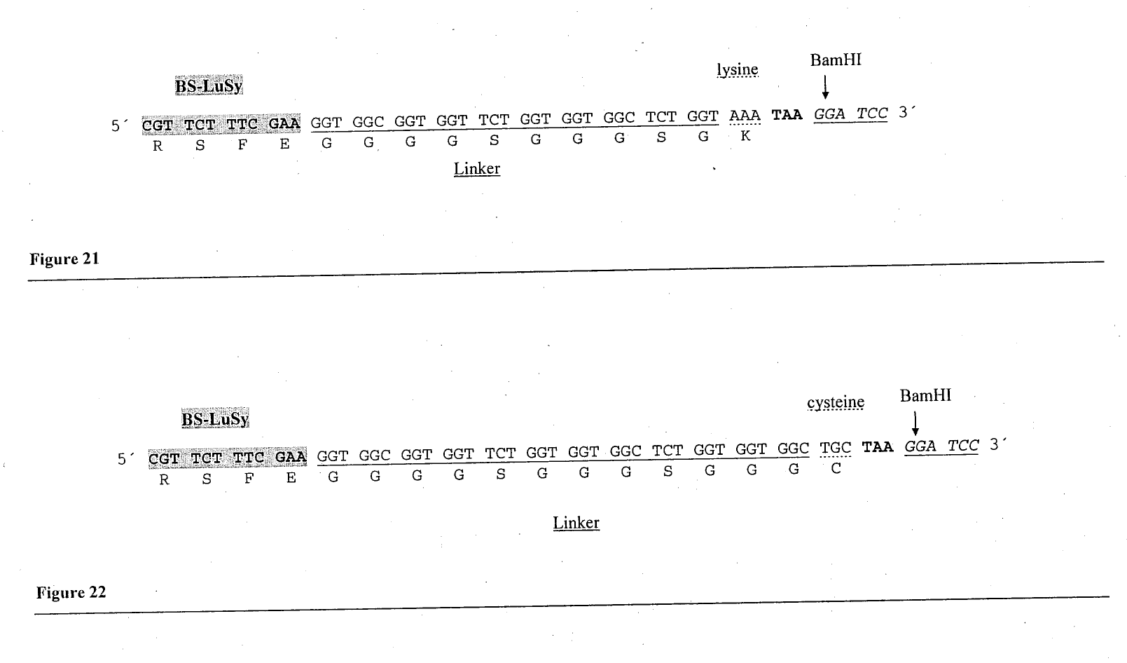

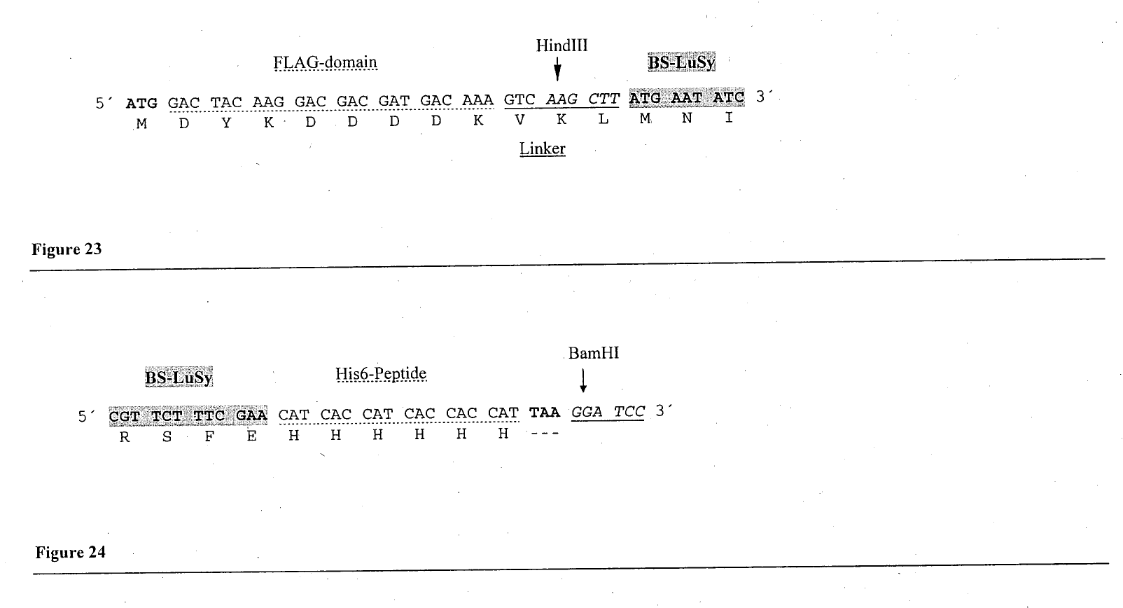

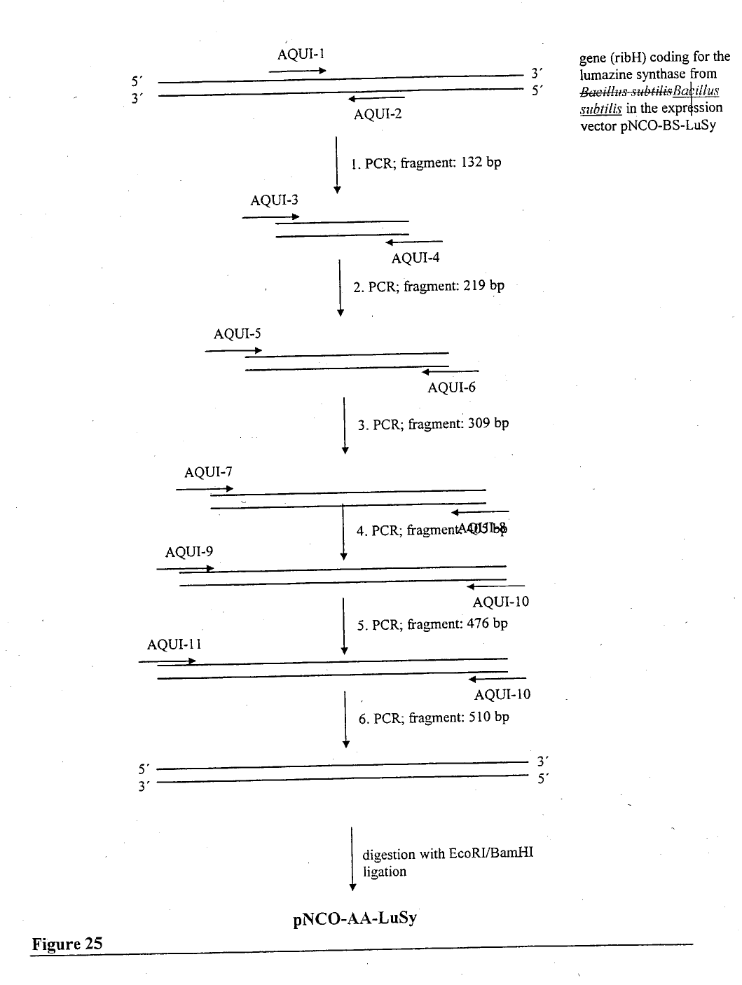

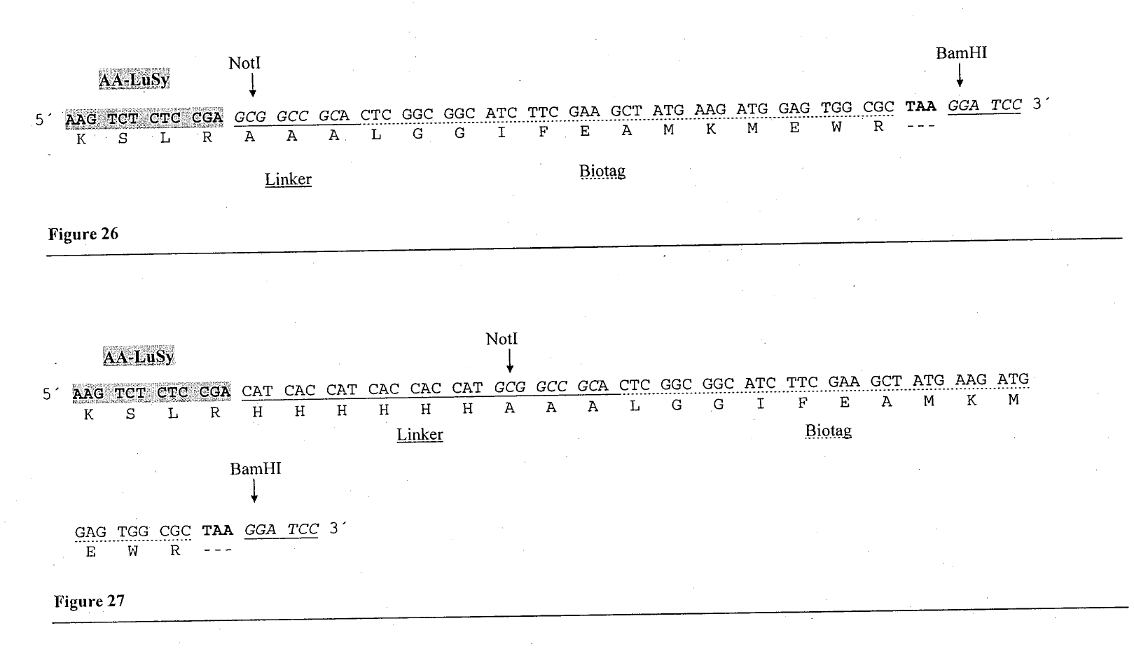

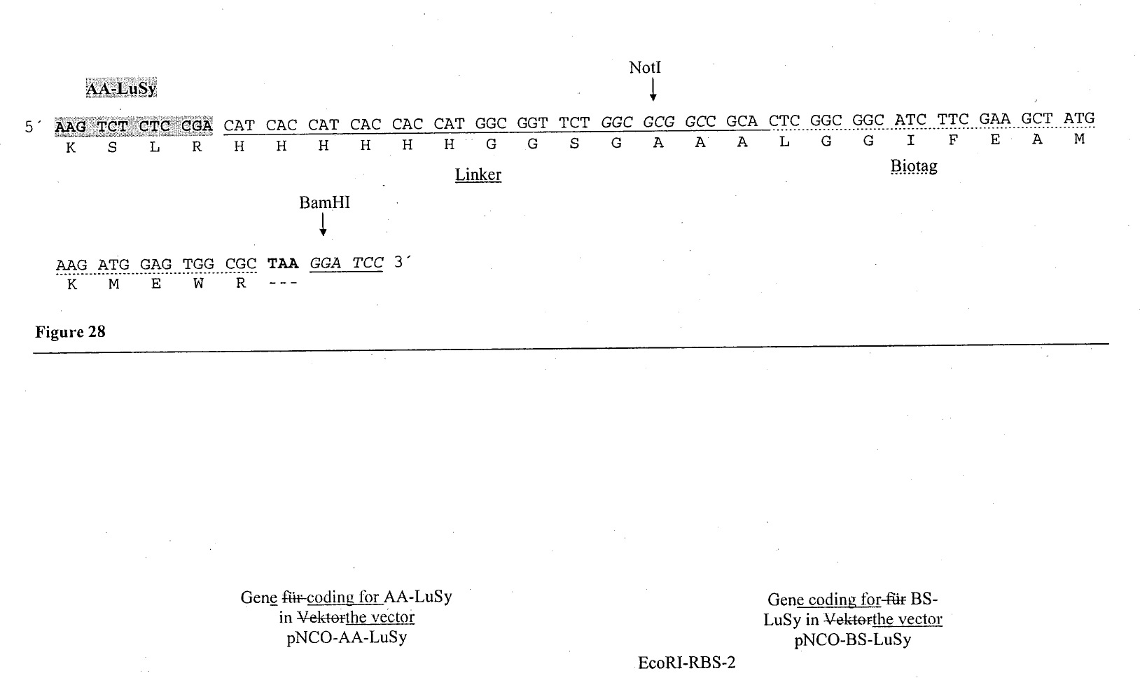

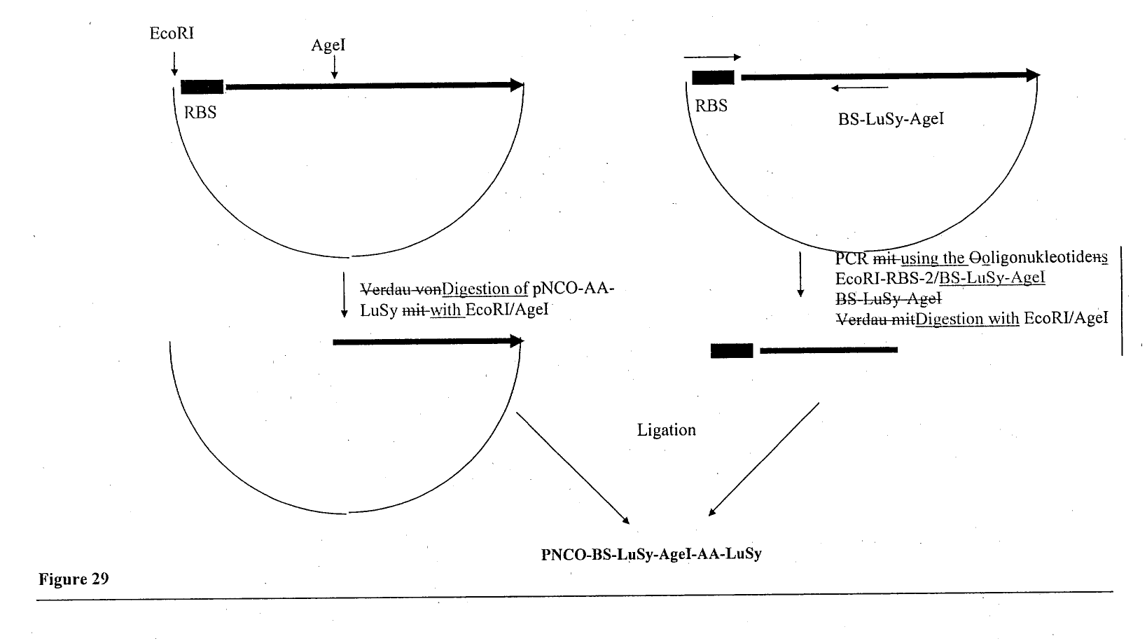

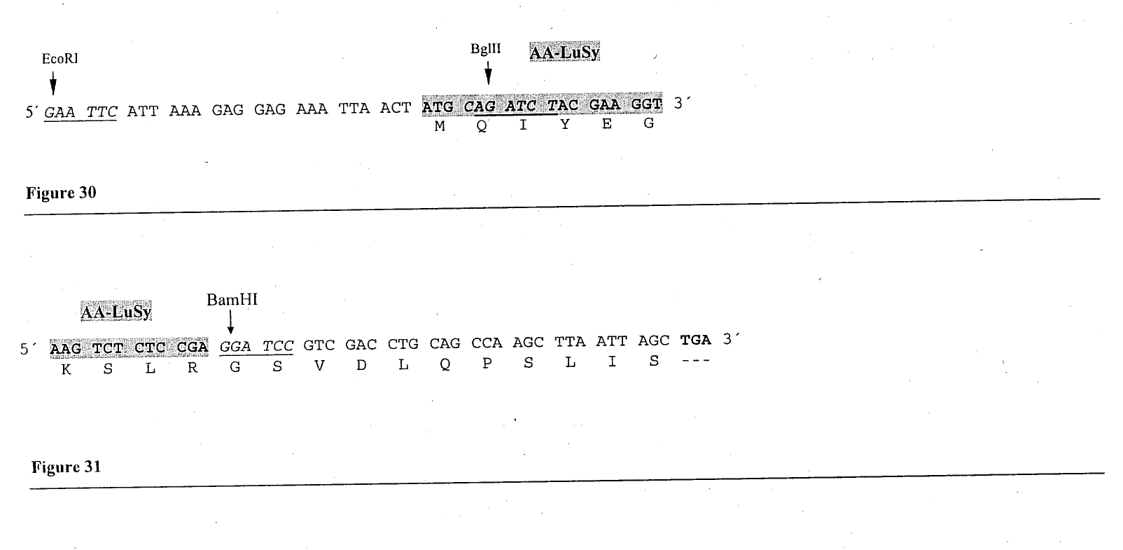

A) A lumazine synthase molecule carrying 1-5 short peptides from antigenically active viral or bacterial surface proteins (antigenic determinants, epitops) and up to 60 biotin molecules in covalent linkage serves as detection molecule for immobilized antibodies which stem from a patient's serum or other fluid.

B) Characteristic antibodies directed against specific infectious diseases are harvested by means of special immobilized epitopes (parts of surface proteins of the respective pathogenic organisms; antigenic determinants) from the respective body fluid. A lumazine synthase molecule which also contains 1-5 copies of the said epitopes and up to 60 biotin molecules in covalent linkage serves as detector molecule for these hereby immobilized antibodies.

A color reaction is obtained in both cases by an arbitrarily selected streptavidine coupled enzyme (E) which forms a complex with the biotinylated lumazine synthase. Through the interposition of a multiply biotinylated linker protein (lumazine synthase conjugate) and the multiple binding of color reaction mediating enzyme mediated hereby, a signal amplification is obtained.

Non-bound antibodies are removed in a first washing step. If no binding to the immobilized epitopes occurs, the complex of lumazine synthase conjugate and antibody is not formed. Excessive lumazine synthase conjugate is removed in a second washing step, such that the assay mixture remains colorless.

FIG. 4 describes a schematic representation of an experimental setup for the purification of antibodies characterized by a specific antigen recognition. A lumazine synthase conjugate comprising a desired foreign peptide (in multiple form) as well as a biotinylated moiety (in singular form) is bound to immobilized streptavidine via its biotin moiety. The streptavidine molecules are coupled to a solid phase. The mixed antibody population is applied to a column of immobilized streptavidine (or is mixed with streptavidine material), whereby the antibodies with the desired specificity bind to the foreign peptide moiety of the lumazine synthase conjugate. The washing process of the streptavidine lumazine synthase conjugate/antibody complex and the subsequent elution of the specific antibodies occurs by known standard methods.

FIG. 5 shows a systematic representation of the structure of a biosensor which can consist, in principle, of three parts: 1. The biological receptor, 2. The transducer unit, 3. The integrated electronic unit. The biological receptor can be linked to the transducer in various ways.

FIG. 6 shows a functionalized lumazine synthase with 60 identical respectively differently modified subunits bound to a surface (for example transducer surface, membrane, other surface etc.) via an anchor (peptide, fatty acid, other functional group etc.). The detection sensitivity for binding of foreign molecules at the surface of the lumazine synthase is enhanced by the large number of functional groups. (for example epitopes for antibody recognition, antibodies for detection of foreign molecules in solution or other receptors).

FIG. 7 schematically shows a possible structure of a field effect transistor under inclusion of a multimeric functionalized lumazine synthase. A modification of the surface charge of the gate electrode resulting from the binding of a foreign molecule to the surface of the lumazine synthase hereby modulates the flux of current through the field effect transistor.

FIG. 8 shows a sequence comparison of lumazine synthases from the following organisms: 1. Mycobacterium avium; 2. Mycobacterium tuberculosis; 3. Corynebacterium ammoniagenes; 4. Chlorobium tepidum; 5. Aquifex aeolicus; 6. Thermotoga maritima; 7. Bacillus subtilis; 8. Bacillus amyloliquefaciens; 9. A. pleuropneumoniae; 10. Streptococcus pneumoniae; 11. Staphylococcus aureus; 12. Vibrio cholerae; 13. Photobacterium phosporeum; 14. S. putrefaciens; 15. Photobacterium leiognathi; 16. Shigella flexneri; 17. Escherichia coli; 18. Haemophilus influenzae; 19. Dehalospirillum multivorans; 20. Helicobacter pylori; 21. Deinococcus radiodurans; 22. Synechocystis sp., 23. Porphyromonas gingivalis; 24. Arabidopsis thaliana; 25. Methanococcus jannaschii; 26. Archaeoglobus fulgidus; 27. Methanobacterium thermoautotrophicum, 28. Chlamydia trachomatis; 29. Saccharomyces cerevisiae; 30. Brucella abortus. The protein sequences were obtained by translation of the cognate DNA sequences. The set of sequences shown was obtained by database search using the search algorithm according Altschul et al. (1997) and the sequence of lumazine synthase of Bacillus subtilis a search motif.

FIG. 9 shows a top view of the pentameric subunit of the icosahedral lumazine synthase of Bacillus subtilis. The ligand 5-nitro-6-ribitylamino-2,4(1H,3H)-pyrimidinedione binds to the contact site between two monomeric subunits (Ladenstein et al., 1988, 1994; Ritsert et al., 1995).

FIG. 10 shows a model of the icosahedral lumazine synthase of Bacillus subtilis. One out of 12 pentameric subunits is emphasized by the use of different gray tones. The N-terminus as well as the C-terminus are located at the surface and are readily accessible.

FIG. 11 shows the expression vectors used in the application examples. SD, ribosomal binding site; MCS, cloning cassette with singular cutting sites; t₀, t₁, terminator sequences; (cat), inactive gene for chloramphenicol acetyl transferase (shifted reading frame); (Δcat), inactive gene for chloramphenicol acetyl transferase (deletion); restriction sites are indicated by letters: B, BamHI; E, EcoRI; H, HindIII; N, NcoI; P, PstI; S, SalI. Translation start in vector pNCO 113 at position 113 and at position 233 for vector p602/-CAT.

FIG. 12 describes the 1. PCR for introduction of a mutation using, as an example, the introduction of a mutation of the amino acid cystein in position 93 against serine in the gene for lumazine synthase of Bacillus subtilis. In the first step of the directed mutagenesis, initially, two separate PCR reactions were performed with the oligonucleotides pairs PNCO-M1/C93S and PNCO-M2/RibH-3 and the expression plasmid pNCO-BS-Lusy as template. Fragment A contains the desired mutation and an intact recognition sequence for the restriction nuclease EcoRI. Fragment B represents the entire, but non-mutagenized ribH gene (lumazine synthase of Bacillus subtilis). In this fragment, the 5′ restriction cloning site is deleted. (R: ribosomal binding site)

FIG. 13 describes the 2. PCR for introduction of a mutation. In the second step of the mutagenesis, the mutation to be introduced which is now still at the 3′ end of the PCR-generated gene fragment is introduced into the entire gene by overlapping elongation.

FIG. 14 describes the 3. PCR for introduction of a mutation. The 3. PCR serves the amplification of the elongated codon strand of fragment A.

In the FIGS. 15-24, 26-28 and 30 and 31, the sequence of the respective lumazine synthases is emphasized by bold type. The linker regions are underlined. Recognition sequences for the respective restriction endonucleases are italicized and underlined. Fused sequences respectively amino acids which are not part of the linker sequence are marked by punctuated underlining. The amino acid sequence is given in the one letter code.

FIG. 15 shows the structure of the vector pNCO-N-BS-LuSy for the fusion of foreign proteins to the N-terminus of lumazine synthase.

FIG. 16 shows the structure of the vector pNCO-C-BS-LuSy for the fusion of foreign proteins to the C-terminus of lumazine synthase.

FIG. 17 shows the structure of the vector pNCO-BS-LuSy-EC-DHFR.

FIG. 18 shows the structure of the vector pNCO-N-VP2-BS-LuSy in the region of the N-terminus.

FIG. 19 shows the structure of the vector pNCO-C-VP2-BS-LuSy in the region of the C-terminus.

FIG. 20 shows the structure of the vector pNCO-C-Biotag-BS-LuSy in the region of the C-terminus.

FIG. 21 shows the structure of the vector pNCO-Lys165-BS-LuSy in the region of the C-terminus.

FIG. 22 shows the structure of the vector pNCO-Cys167-BS-LuSy in the region of the C-terminus.

FIG. 23 shows the structure of the vector pFLAG-MAC-BS-LuSy in the region of the N-terminus.

FIG. 24 shows the structure of the vector pNCO-C-His6-BS-LuSy in the region of the C-terminus.

FIG. 25 shows the construction of the thermostable lumazine synthase of Aquifex aeolicus (Deckert et al., 1998) using 11 synthetic oligonukleotides (AQUI-1 tos AQUI-11) and 6 steps of polymerase chain reaction.

FIG. 26 shows the coupling of an artificial peptide with a length of 13 amino acids, which is accessible to in vivo biotinylation, to the C-terminus of the thermostable lumazine synthase of Aquifex aeolicus. (The peptide is bound to the C-terminus of the carrier protein by a linker of 3 alanine residues)

FIG. 27 shows the coupling of an artificial peptide with the length of 13 amino acids, which is accessible to in vivo biotinylation, to the C-terminus of the thermostable lumazine synthase of Aquifex aeolicus by a linker of 6 histidine and 3 alanine residues.

FIG. 28 shows the coupling of an artificial peptide with a length of 13 amino acids, which is accessible to in vivo biotinylation, to the C-terminus of the thermostable lumazine synthase of Aquifex aeolicus by a linker consisting of 6 histidine residues and the sequence Gly-Gly-Ser-Gly-Ala-Ala-Ala

FIG. 29 shows the production of a chimeric protein consisting of a part of the lumazine synthase of Bacillus subtilis and a part of the thermostable lumazine synthase of Aquifex aeolicus

FIG. 30 shows the 5′ region of the vector pNCO-AA-BglII-LuSy respectively the vector pNCO-AA-BglII-LuSy-(BamHI) for the fusion of foreign genes to the 5′ end of lumazine synthase of Aquifex aeolicus. The recognition sequence for the singular restriction nuclease BglII newly introduced into the sequence is marked.

FIG. 31 shows the 3′ region of vector pNCO-AA-BgIII-LuSy respectively pNCOAA-BglII-LuSy-(BamHI) for fusion of foreign genes to the 3′ end of lumazine synthase of Aqufex aeolicus. A peptide with the sequence GSVDLQPSLIS is fused to the C-terminus of the sequence.

The describes DNA sequence protocols illustrate the structure of the plasmids shown in the examples. In the sequence protocols, the recognition sequences of the respective restriction endonucleases used are underlined and italicized; the expressed fusion proteins are shown in bold type, and linker sequences are shown is punctuated underlining; exceptions in the formatting are indicated.

SEQ ID No.1 shows the DNA sequence of the expression vector pNCO113 (vector for expression of genes in Escherichia coli; Stüber et al., 1990).

SEQ ID No.2 shows the DNA sequence of the expression vector p602/-CAT (shuttle vector for expression of genes in Escherichia coli and Bacillus subtilis; Henner, 1990; LeGrice, 1990).

SEQ ID No.3 shows the DNA sequence of the expression plasmid pNCO-BS-LuSy (expression plasmid with an unmodified lumazine synthase of Bacillus subtilis for expression in Escherichia coli).

SEQ ID No.4 shows the DNA sequence of the expression plasmid p602-BS-LuSy (expression plasmid with an unmodified lumazine synthase of Bacillus subtilis for expression in Escherichia coli and Bacillus subtilis).

SEQ ID No.5 shows the DNA sequence of the expression plasmid pNCO-BS-LuSy-C93S (expression plasmid with a modified lumazine synthase variant, whereby the amino acid cystein in position 93 was exchanged by the amino acid serin).

SEQ ID No.6 shows the DNA sequence of the expression plasmid pNCO-BS-LuSy-C139S (expression plasmid with a modified lumazine synthase variant, whereby the amino acid cystein in position 139 was exchanged by the amino acid serin).

SEQ ID No.7 shows the DNA sequence of the expression plasmid pNCO-BS-LuSy-C93/139S (expression plasmid with a modified lumazine synthase variant, whereby the amino acid cystein in positions 93 and 139 was exchanged by the amino acid serin).

SEQ ID No.8 shows the DNA sequence of the expression vector pNCO-N-BS-LuSy for the fusion of foreign peptides to the N-terminus of the lumazine synthase of Bacillus subtilis.

SEQ ID No.9 shows the DNA sequence of the expression vector pneCO-C-BS-LuSy for the fusion of foreign peptides to the C-terminus of the lumazine synthase of Bacillus subtilis.

SEQ ID No.10 shows the DNA sequence of the expression vector pNCO-EC-DHFR-BS-LuSy (expression plasmid for expression of a fusion protein consisting of dihydrofolate reductase of Escherichia coli and the lumazine synthase of Bacillus subtilis, whereby the dihydrofolate reductase is fused to the N-terminus of lumazine synthase).

SEQ ID No.11 shows the DNA sequence of the expression vector pNCO-EC-MBP-BS-LuSy. (expression plasmid for expression of a fusion protein comprising maltose binding protein of Escherichia coli and the lumazine synthase of Bacillus subtilis, whereby the maltose binding protein is fused to the N-terminus of lumazine synthase). SEQ ID No.12 shows the DNA sequence of the expression vector pNCO-BS-LuSy-EC-DHFR. The linker sequence between the lumazine synthase and the dihydrofolate reductase is underlined in punctuated lines. (Expression plasmid for expression of a fusion protein consisting of the dihydrofolate reductase of Escherichia coli and the lumazine synthase of Bacillus subtilis whereby the dihydrofolate reductase is fused to the C-terminus of the lumazine synthase).

SEQ ID No.13 shows the DNA sequence of the expression vector pNCO-N-VP2-BS-LuSy. (Expression plasmid for expression of a fusion protein consisting of the VP2-domain of the “Mink enteritis virus” and the lumazine synthase of Bacillus subtilis, whereby the VP2-domain is located at the N-terminus; the pristine start codon of the lumazine synthase is underlined).

SEQ ID No.14 shows the DNA sequence of the expression vector pNCO-C-VP2-BS-LuSy. (Expression plasmid for expression of a fusion protein consisting of the VP2-domain of the “Mink enteritis virus” and the lumazine synthase of Bacillus subtilis, whereby the VP2-domain is located at the C-terminus).

SEQ ID No.15 shows the DNA sequence of the expression vector pNCO-N/C-VP2-BS-LuSy. (Expression plasmid for expression of a fusion protein consisting of the VP2-domain of the “Mink enteritis virus” and the lumazine synthase of Bacillus subitlis, whereby the VP2-domain is located at the N-terminus as well as at the C-terminus; the pristine start codon of the lumazine synthase is underlined).

SEQ ID No.16 shows the DNA sequence of the expression vector pNCO-C-Biotag-BS-LuSy. (Expression plasmid for expression of a fusion protein consisting of a peptide consisting of 13 amino acids which is susceptible to biotinylation in vivo, and of the lumazine synthase of Bacillus subitlis, whereby the fused peptide is located at the C-terminus).

SEQ ID No.17 shows the DNA sequence of the expression vector pNCO-Lys165-BS-LuSy. (Expression plasmid for expression of a modified lumazine synthase of Bacillus subitlis, whereby the C-terminus has been elongated and ends with a lysine residue; the codon for lysine (AAA) is underlined).

SEQ ID No.18 shows the DNA sequence of the expression vector pNCO-Cys167-BS-LuSy. (Expression plasmid for expression of a modified lumazine synthase of Bacillus subitlis, whereby the C-terminus has been elongated and ends with a cystein residue; the codon for cystein (TGC) is underlined).

SEQ ID No.19 shows the DNA sequence of the expression vector pFLAG-MAC-BS-LuSy. (Expression plasmid for expression of a fusion protein comprising an epitope which consists of 12 amino acids that can be recognized by a monoclonal antibody, as well as the lumazine synthase of Bacillus subtilis, whereby the fused peptide is located at the N-terminus; the pristine start codon of the lumazine synthase is underlined).

SEQ ID No.20 shows the DNA sequence of the expression vector pNCO-C-His6-BS-LuSy. (Expression plasmid for expression of a fusion peptide comprising a peptide with the length of six amino acids (6×histidine) and the lumazine synthase of Bacillus subtilis, whereby the fused peptide is located at the C-terminus; the peptide is underlined).

SEQ ID No.21 shows the DNA sequence of the expression vector pNCO-AA-LuSy. (Expression plasmid for expression of the unmodified, thermostable lumazine synthase of Aquifex aeolicus; the DNA sequence has been adapted to the codon usage of Escherichia coli; the DNA has been synthesized in its entirety).

SEQ ID No.22 shows the DNA sequence of the expression vector pNCO-C-Biotag-AA-LuSy. (Expression plasmid for expression of a fusion protein comprising a peptide with the length of 13 amino acids which is susceptible to biotinylation, and the lumazine synthase of Aquifex aeolicus, whereby the fused peptide is located at the C-terminus; the peptide is connected to the C-terminus of the carrier protein by a linker of 3 alanine residues).

SEQ ID No.23 shows the DNA sequence of the expression vector pNCO-His6-C-Biotag-AA-Lusy. (Expression plasmid for the expression of the lumazine synthase of Aquifex aeolicus with a C-terminal peptide which is susceptible to in vivo biotinylation and which is coupled via a linker of 6 histidine and 3 alanine residues).

SEQ ID No.24 shows the DNA sequence of the expression vector pNCO-His6-GLY2-SER-GLY-C-Biotag-AA-LuSy. (Expression plasmid for the expression of the lumazine synthase of Aquifex aeolicus with a C-temrinal peptide which is susceptible to in vivo biotinylation and which is coupled via a linker with the amino acid sequence HHHHHHGGSGAAA).

SEQ ID No.25 shows the DNA sequence of the expression vector pNCO-BS-LuSy-AgeI-AA-LuSy. (Expression plasmid for expression of a chimeric protein consisting a part of lumazine synthase of Bacillus subtilis and a part of the thermostable lumazine synthase of Aquifex aeolicus; the Bacillus subtilis lumazine synthase part is shown in bold type, the Aquifex aeolicus lumazine synthase part is double underlined).

SEQ ID No.26 shows the DNA sequence of the expression vector pNCO-AA-BglII-LuSy (Vector for fusion of foreign peptides to the N-terminus respectively to the 5′ end of the thermostable lumazine synthase of Aquifex aeolicus using the restriction endonuclease BglII).

SEQ ID No.27 shows the DNA sequence of the expression vector pNCO-AA-LuSy-(BamHI). (Vector for fusion of foreign peptides to the C-terminus respectively to the 3′ end of the thermostable lumazine synthase of Aquifex aeolicus using the restriction endonuclease BamHI).

SEQ ID No.28 shows the DNA sequence of the expression vector pNCO-AA-BglII-LuSy-(BamHI). (Vector for fusion of foreign peptides to the N-terminus and the C-terminus respectively to the 5′ and 3′ ends of the thermostable lumazine synthase of Aquifex aeolicus using the restriction endonuclease BamHI).

### EXAMPLES

### Example 1

Heterologous Expression of the Gene (ribH) Coding for the Iumazine Synthase from Bacillus subtilis in Escherichia coli XL1 Cells

A) The gene coding for the lumazine synthase from Bacillus subtilis was amplified using the oligonucleotide RibH-1 (5′ gag gag aaa tta acc atg aat atc ata caa gga aat tta g 3′) as forward primer, which was at his 3′-end identical to the 5′-end of the ribH gene and which coded for an optimized ribosome binding site at his 5′-end. As reverse primer the oligonucleotide RibH-2 (5′ tat tat gga tcc cca tgg tta ttc gaa aga acg gtt taa gtt tg 3′) was used, which was at his 3′-end identical to the 3′-end of the ribH gene and which introduced a recognition site for the restriction endonuclease BamHI (G*GATCC) in close distance to the stop codon. The plasmid pRF2 (Perkins et al., 1991) was used as template for the PCR (Mullis et al., 1986).

10 μl PCR-buffer (75 mM Tris/HCl, pH 9.0; 20 mM (NH₄)₂SO₄; 0.01% (w/v) Tween 20)

6 μl Mg²⁺[1.5 mM]

8 μl dNTP's [each 200 μM]

1 μl RibH-1 [0.5 μM]

1 μl RibH-2 [0.5 μM]

1 μl pRF2 [10 ng]

1 μl Goldstar-Taq-Polymerase [0.5 U] (Eurogentec, Seraing, Belgien)

72 μl H₂O_{bidest}

PCR cycle protocol (GeneAmp® PCR System 2400; Perkin Elmer):

1. 5.0 min 95° C.

2. 0.5 min 94° C.

3. 0.5 min 50° C.

4. 0.8 min 72° C.

5. 7.0 min 72° C.

6. ∞ 4° C.

Steps 2.-4. were repeated 20 times.

B) The PCR mixture was analyzed and separated on an agarose gel, the DNA was visualized using ethidium bromide and UV light and the DNA fragment with a length of 498 bp was isolated from the gel. The DNA fragment was purified using the Geneclean II-Kit from Bio101 (San Diego, Calif., USA) according to the manufacture's instructions. In the last step the DNA was eluted using 30 μl bidest. water and a incubtion temperature of 45° C. for 15 min. The concentration of the DNA was measured by fluorescense spectroscopy using the intercalation dye bisbenzimide H 33258 (Höchst, Frankfurt, Germany). The measuring was carried out by an excitation of 365 nm, and emission of 458 nm. The blank was measured with 2 ml of TNE buffer (100 mM Tris/HCl pH 7.4, 10 mM EDTA, 1 M NaCl) which contained 0.1 μg/ml H 33258. 2 μl plasmid DNA with known concentration was used as DNA standard for the calibration.

C) 10 ng of the purified DNA from B) served as a template for a 2. PCR. using the oligonucleotide EcoRI-RBS-1 (5′ ata ata gaa ttc att aaa gag gag aaa tta acc atg 3′), which was identical to the 5′-end of primer RibH-1 and which extended the ribosome binding site in 5′-direction. In close 5′ contact to the ribosome binding site, a recognition site for the endonuclease EcoRI (G*AATTC) was introduced into the DNA fragment. The oligonucleotide RibH-2 was used as reverse primer.

10 μl PCR-buffer

6 μl Mg²⁺[1.5 mM]

8 μl dNTP's [each 200 μM]

1 μl EcoRI-RBS [0.5 μM]

1 μl RibH-2 [0.5 μM]

1 μl DNA from B) [10 ng]

1 μl Goldstar-Taq-Polymerase [0.5 U] (Eurogentec, Seraing, Belgien)

72 μl H₂O_{bidest}

PCR cycle protocol (GeneAmp® PCR System 2400; Perkin Elmer):

1. 5.0 min 95° C.

2. 0.5 min 94° C.

3. 0.5 min 50° C.

4. 0.8 min 72° C.

5. 7.0 min 72° C.

6. ∞ 4° C.

Steps 2.-4. were repeated 20 times.

D) The PCR mixture was analyzed and separated on agarose gel and a DNA fragment with a length of 516 bp was isolated according to B).

E) The isolated DNA-fragment was digested using the restriction endonucleases EcoRI and BamHI.

30.0 μl DNA from D)

2.5 μl EcoRI [62.5 U]

3.0 μl BamHI [60 U]

24.0 μl OPAU (10×; 500 mM pottasium acetate; 100 mM magnesium acetate; 100 mM tris-acetate, pH 7.5)

60.5 μl H₂O_{bidest}

The enzymes were purchased from Pharmacia Biotech (Freiburg, Germany). The mixture was incubated for 180 min at 37° C. After the incubation the mixture was purified according to B) and used in a ligation protocol.

F) The expression vector was digested using the restriction endonucleases EcoRI and BamHI.

25.0 μl pNCO113 [5 μg]

2.5 μl EcoRI [62.5 U]

3.0 μl BamHI [60 U]

24.0 μl OPAU (10×)

65.5 μl H₂O_{bidest}

The enzymes were purchased from Pharmacia Biotech (Freiburg, Germany). The mixture was incubated for 180 min at 37° C. After the incubation the mixture was purified according to B) and used in a ligation protocol.

G) The DNA fragments resulting from E) and F) were ligated in a molecular relation of 3 to 1 (Sgamarella, 1979).

1 μl expression vector from F) [50 fmol]

2 μl DNA-fragment from E) [150 fmol]

4 μl H₂O_{bidest}

mix, 10 min/55° C., 5 min on ice

2 μl T₄-Puffer (5×; 250 mM tris/HCl, pH 7.6; 50 mM MgCl₂; 5 mM ATP; 5 mM DTT;

25% (w/v) polyethylene glycole-8000)

1 μl T₄-Ligase [1 U] (Gibco BRL, Eggenstein, Germany)

The mixture was incubated at 4° C. overnight yielding the plasmid pNCO-BS-LuSy.

H) Preparation of electrocompetent Escherichia coli XL1-cells (Dower et al., 1988) and electroporation: 1 liter LB-medium (10 g/l peptone; 5 g/l yeast extract; 5 g/l NaCl) was inoculated with 10 ml of a XL1 cell suspension which was grown overnight at 28° C. The cell culture was then incubated in a incubator under shaking at 37° C. At an optical density (600 nm) of 0.5 to 0.7 the culture was placed on ice for 15 min. Cells were harvested by centrifugation (Sorvall-GS-3-Rotor, 2300 rpm, 4° C., 15 min). The cell pellet was suspended in 1 liter sterile glycerol solution (10% in water, w/w) and the mixture was centrifuged again using the same conditions. The resulting pellet was then washed with 500 ml glycerol solution, centrifuged and at least washed with 20 ml glycerol solution and centrifuged again. After the last centrifugation step the pellet was suspended in 2-3 ml of glycerol solution and placed on ice (electrocompetent cells). The electroporation tube (0,1 cm) and the tube holder were cooled on ice for 15 min. 40 μl of electrocompetent cells were mixed with 1-2 μl of the ligation mixture from G) in a precooled 1.5 ml cap and after that transferred to the precooled electroporation tube. The electroporation was carried out in a electroporation device from Biorad (Munich, Germany). Conditions: 25 μF, 1.8 kV, 200 Ω. After the pulse the suspension was mixed with 1 ml of SOC medium (2% peptone; 0.5% yeast extract; 10 mM NaCl; 2.5 mM KCl; 10 mM MgCl₂; 10 mM MgSO₄; 20 mM glucose). The transformation mixture was then incubated for 1 h at 37° C. in a shaker. After this step 20 μl and 200 μl aliquots were plated on LB-Amp-agar-plates (21 g/l Agar; 10 g/l peptone; 5 g/l yeast extract; 5 μl NaCl; 150 mg/l ampicilline) and incubated overnight at 37° C. resulting in the expression strain XL1-pNCO-BS-LuSy.

I) A plasmid (pNCO-BS-LuSy) form H) was isolated using the method described by Birnboim und Doly (1979). Cells from a 100 ml overnight culture were suspended in 4 ml of buffer S1 (50 mM tris/HCl, 10 mM EDTA, 100 μg RnaseA/ml, pH 8.0) and then 4 ml of buffer S2 (200 mM NaOH, 1% SDS) was added. After gentle shaking of the suspension and 5 min incubation at room temperature, 4 ml of buffer S3 (2.6 M KAc, pH 5.2) was added. The resulting mixture was incubated for 20 min on ice. After centrifugation (Sorvall-SS34-Rotor, 17000 rpm, 4° C., 30 min) the supernatant was placed on a Nucleobond® AX100 column (Macherey und Nagel, Düren) which was equilibrated with 2 ml of buffer N2 (0.9 M KCl; 100 mM tris-phosphate, pH 6.3; 15% (v/v) ethanol). The column was washed using 8 ml buffer N3 (1.3 M KCl, pH 6.3). After that the DNA was eluted using 2 ml buffer N5 (1.3 M KCl, pH 8.0). The DNA was precipitated using 1.4 ml isopropanole and the DNA-pellet was washed twice with icecold ethanol (70% in water, (v/v)). After that the pellet was dried in a vacuum centrifuge and the resulting DNA-pellet was solved in 200 μl bidest. water.

J) The isolated plasmid (pNCO-BS-LuSy) from I) was sequenced using the chain termination method from Sanger et al. (1971). The sequencing mixture contained 1 μg plasmid-DNA from I), 10 pmol sequencing primer Seq-1 (5′ gtg agc gga taa caa ttt cac aca g 3′), 10 μl terminator Premix™ (dNTP's, ddNTP's, labeled ddNTP's und Taq-DNA-polymerase) from ABI (Weiterstadt, Germany) and bidest. water to a endvolume of 21 μl. The reaction was carried out in a GeneAmpPCR System 2400 device from Perkin Elmer (Norwalk, Conn., USA).

PCR cycle protocol:

15 s/96° C.

15 s/50° C.

4 min/72° C.

The PCR steps were repeated 20 times.

In a following step 80 μl bidest. water was added and the mixture was shaked out two times using 100 μl phenole/chloroform/amylalcohole-mix (25:24:1) from ABI (Weiterstadt, Germany). The DNA was pecipitated with 300 μl ethanol containing 10 μl 3 M Na-acetate. The suspension was then centrifuged (14000 rpm, RT, 30 min) and the resulting pellet was washed with ethanol (70%, v/v, ice cooled) and dried in a vacuum centrifuge. The DNA was then solved in a solution containing 1 μl 50 mM EDTA, pH 8.0 and 5 μl formamide. The DNA was incubated 2 min at 95° C. and then cooled on ice. 1.5 μl of this solution was placed on a 4.75% polyacrylamide-sequencing gel. Preparation of the polyacrylamide gel: 13.3 ml of UltraPureSequagel™Sequencing-System-conzentrate from National Diagnostics (Atlanta, Ga., USA) was mixed with 49.7 ml UltraPureSequagel™Sequencing-System-Diluent and deionized with Amberlite MB-1. The suspension was filtrated (0.2 μm) and 7 ml UltraPureSequagel™Sequencing-System-buffer was added. After deairing, 210 μl ammonium peroxodisulfate-solution (10%, w/w) and 25 μl TEMED were added. The mixture was placed in a gel tray. The developing of the gel was carried out in TBE buffer (1 M Tris-Base, pH 8.3; 0.85 M Boron acid; 10 mM EDTA) using a Prism™377-DNA-Sequencer from Perkin-Elmer-ABI (Weiterstadt, Germany).

K) Expression strains containing an expression plasmid from I) were fermented in 25 ml LB-AMP-medium (10 g/l peptone; 5 g/l yeast extract; 5 g/l NaCl; 150 mg/l ampicilline). The culture was inoculated with 500 μl of an overnight culture from H) (relation: 1:50 (v/v)). After an optical density (600 nm) of 0.7 the expression was induced by the addition of IPTG (isopropyl-β-D-thiogalactopyranoside) resulting in a final concentration of 2 mM. At an additional incubation of 5 h, cells were harvested by centrifugation (5000 rpm 4° C., 15 min). The pellet was washed with 5 ml 0.9% NaCl (w/v) (20% of the culture volume) and stored at −20° C.

L) Cells from K) were thawed and lysed using an ultrasonic device from Branson SONIC Power Company (Branson-Sonifier B-12A, Branson SONIC Power Company, Dunbury, Conn., USA). The cell pellet from K) suspended in 800 μl lysis-buffer (50 mM K-phosphate, pH 7.0; 10 mM EDTA; 10 mM Na₂SO₃; 0.3 mM PMSF; 0.02% Na-azide) and incubated for 10 min on ice. The cell suspension was then lysed using the ultrasonic device (one pulse for 8 sec and level 4.5). The suspension was then cooled on ice for 5 min and lysed under the same conditions for a second time. After the second sonication the suspension was centrifuged (Eppendorff-centrifuge; 15000 rpm, 4° C., 15 min) and the supernatant (crude lysate) was used for the following steps.

M) To check the expression level and the molecular weight of the monomeric subunit of the expressed lumazine synthase a SDS gel electrophoresis (SDS-PAGE) according to Laemmli (1970) was carried out. As a matter of routine gels with 4% acrylamide in the collecting gel and 16% acrylamide in the separating gel were prepared (acrylamide stocksolution: 38.8% (w/v) acrylamide; 1.2% (w/v) N,N′-methylene bisacrylamide). The crude lysate from L) has been diluted 1:2, 1:5 and 1:10 with sample buffer (20% glycerol; 4% 2-mercapto ethanol; 4% (w/v) SDS; 0.05% bromphenolblue) and boiled for 15 min. After cooling down the samples were centrifuged (15000 rpm, 5 min, 4° C.) and 8 μl of the clear supernatant were used for the SDS-PAGE. As molecular weight standard we used Dalton Mark VII-L from Sigma (Deisenhofen, Germany) containing marker proteins with molecular weights of 66, 44, 36, 29, 24, 20 und 14 kDa (Standard proteins). The electrophoresis was carried out by a constant voltage of 20 mV. After the development of the gel it was stained with coomassie blue dye (40% methanol; 10% acetic acid; 0.2% (w/v) coomassie Blue R 250). To remove the dye out of the polyacryl amide gel (not out of the protein) a dye removing solution was used (40% methanol; 10% acetic acid; 50% water). In the crude lysate of the strain XL1-pNCO-BS-LuSy a protein band with a molecular weight of circa 16 kDa could be observed. This protein band couldn't be observed in a Escherichia coli strain without the expression plasmid pNCO-BS-LuSy. The observed protein band corresponded to circa 10% of the total soluble proteins of the Escherichia coli strain.

N) To check the enzymatic function of the protein an enzyme assay using the native substrates 5-amino-6-ribitylamino-2,4(1H,3H)-pyrimidindione and L-3,4-dihydroxy-2-butanone-4-phosphate; Bacher et al., 1997) was carried out. The assay mixture contained 100 mM K-phosphate-buffer pH 7.0, 4 mM EDTA, 0.6 mM 5-amino-6-ribitylamino-2,4(1H,3H)-pyrimidindione (PYR; obtained by catalytic reduction of 5-nitro-6-ribitylamino-2,4(1H,3H)-pyrimidindione), 2 mM DTT, 1 mM L-3,4-dihydroxy-2-butanone-4-phosphate (DHP) and crude lysate from L). In a first step the mixture was incubated (without PYR) for 3 min at 37° C. Afterwards the reaction was started by addition of PYR and incubated at 37° C. After several time intervals (2, 5 and 10 min) aliquots of the mixture were removed and the reaction in those aliquots was stoped by the addition of TCA (15% in water; (w/v)) and centrifuged (15000 rpm, 5 min, room temperature). The quantity of the product of the enzyme reaction (6,7-dimethyl-8-ribityllumazine) was checked by HPLC (column: reverse phase Nucleosil 10C₁₈ (4×250 mm); excitation: 407 nm; emission: 487 nm; elution buffer: 7% methanol, 30 mM formic acid). As standard chemical synthesized 6,7-dimethyl-8-ribityllumazine was used. One unit (1 U) of the enzyme 6,7-dimethyl-8-ribityllumazine synthase catalyzed the formation of 1 nmol 6,7-dimethyl-8-ribityllumazine per hour at 37° C. In the crude lysate of the strain XL1-pNCO-BS-LuSy a volume activity of 15600 U/ml could be measured. After determination of the total protein concentration (13 mg/ml) of the crude lysate according to O) a specific activity of 1200 U/mg could be calculated.

O) The determination of the protein concentration in the crude lysate was carried out using a modified variant of the Bradford-method (Read and Northcote, 1981; Compton and Jones, 1985). The reactive-reagent contained 0.1 g Serva Blue G, 100 ml 16 M phosphoric acid and 47 ml ethanol. The solution was filtrated and stored in the dark at 4° C. The crude lysate was diluted 50-fold with bradford-buffer (2.0 g Na₂HPO₄, 0.6 g KH₂PO₄, 7.0 g NaCl, 0.2 g Na-azide per liter water; (w/v)). 50 μl of the diluted solution was mixed with 950 μl of reactive-reagent and incubated at room temperature for 15 min. Afterwards the extinction of the mixture was determined at 595 nm. Each measurement was carried out three times and summarized to a mean value. As a blank a solution containing 50 μl bradford-buffer and 950 μl reactive-reagent was used. The blank was handled under the same conditions. Each sample was measured three times. For the calibration bovine serum albumin with known concentration was used and the protein concentration of the crude lysate calculated on the basis of the calibration curve.

P) To carry out negative staining experiments on a electron microscope grids coated with formvar/carbon were used. Circa 10 μl protein solution (≈1 mg/ml) were placed on the grid and incubated for 1 min at room temperature. The surplus protein solution which wasn't adsorbed to the grid was removed after the incubation period. Afterwards the grid was incubated with uranyl acetate (30 sec; 2% in water) and washed with water. This procedure was repeated 2-3 times. Subsequent the grid was dried and placed in the grid holder of the electron microscope (JEM-100CX, Jeol, Japan). Negative staining shots showed hollow spherical particles with an outer diameter of circa 15 nm and an inner diameter of circa 5 nm.

Q) The western blot analysis was carried out according to a method from Sambrook et al. (1989). Starting from a denaturing SDS-polyacrylamide gel (16%) proteins were transfered on a PVDF membrane by electro blotting (constant current: 40 mA, 2 h). After the transference of the proteins, the membrane was rinsed in antibody-washing-solution-A (20 mM Tris, pH 7.4; 150 mM NaCl; 3 mM KCl; 0.05% Tween 20). Afterwards the membrane was incubated in antibody-washing-solution-B (antibody-washing-solution-A containing 3% skimmed milk powder) for 1 h at room temperature. Subsequent the membrane was incubated overnight in 5 ml antibody-washing-solution-C (antibody-washing-solution-A containing 1% skimmed milk powder) containing 10 μl Anti-sRFS solution (primary antibody; rabbit crude serum with polyclonal antibodies against lumazine synthase from Bacillus subtilis; diluted 1:10 in antibody-washing-solution-C). Afterwards the membrane was washed 3 times using 5 ml antibody-washing-solution-A. Subsequent the membrane was incubated in 5 ml antibody-washing-solution-C containing 20 μl secondary antibody conjugate (Anti-rabbit-IgG-HRP-conjugate in 50% glycerole; Sigma, Munich, Germany). Afterwards the membrane was washed 3 times using 5 ml antibody-washing-solution-A. The visualization of the lumazine synthase was carried out using the substrates for the horse radish peroxidase 3,3′-diaminobenzidine (6 mg in 10 ml antibody-washing-solution-A) and 10 μl perhydrole (30%). The lumazine synthase could be detected on the membrane as a single band with a molecular weight of circa 16 kDa.

R) The isolation of the lumazine synthase from the Escherichia coli strain XL 1-pNCO-BS-LuSy was carried out in two steps. The fermentation of the cells was carried out according to K), however in a volume of 1 liter. After washing the cells in 0.9% NaCl (w/v; 20% of the culture volume) the pellet was suspended in 32 ml lysis-buffer (L)) and cooled on ice for 10 min. Afterwards the cells were lysed using a ultrasonic device from Branson SONIC Power Company (Branson-Sonifier B-12A, Branson SONIC Power Company, Dunbury, Conn., USA; 15 pulses at level 5). The suspension was then cooled on ice for 5 min and lysed under the same conditions for a second, third and forth time. After the forth sonication the suspension was centrifuged (Sorvall SS34-Rotor; 15000 rpm, 4° C., 15 min) and the supernatant was applied to anion exchange column (DEAE-Cellulose DE52; 2×15 cm, Whatman Ltd., Maidstone, GB) equilibrated with buffer A (50 mM K-phosphate, 10 mM EDTA, 10 mM Na-sulfite, 0.02% Na-azide, pH 7.0). The column was rinsed using 100 ml buffer A. After that the column was developed using a salt gradient from 50 mM phosphate (buffer A) to 1 M phosphate (buffer B: 1 M K-phosphate, 10 MM EDTA, 10 mM Na-sulfite, 0.02% Na-azide, pH 7.0; gradient profile: 101 ml to 200 ml 15% buffer B; 201 ml to 500 ml 18% buffer B; 501 ml to 650 ml 100% buffer B) with a flow rate of 1 ml/min. The lumazine synthase could be eluted at a salt concentration of 250 mM phosphate. The fractions were checked for lumazine synthase activity according to N). Enzymatic active fractions were collected and dialysed against buffer A in a volume ratio of 1:1000 (18 h, 4° C.). The dialysed protein solution was concentrated using an ultra centrifuge (Beckman LE 70 with rotor 70Ti; 32000 rpm, 18 h, 4° C.). The concentrated protein solution (75% pure) was applied to gel filtration column which had been equilibrated with buffer A (Sepharose-6B, 2×180 cm, Pharmacia Biotech, Freiburg, Germany). The column was developed using buffer A (flow rate of 0.5 ml/min). The fractions were checked for lumazine synthase activity according to N). Enzymatic active fractions were collected and concentrated using an ultracentrifuge (Beckman LE 70 with rotor 70Ti; 32000 rpm, 18 h, 4° C.).

S) The purity check was carried out according to M) (SDS-PAGE) whereby only one band could be observed at a molecular weight of circa 16 kDa. The enzymatic activity was measured according to N), the protein concentration was determined according to O). Using these data a specific activity of 12400 U/mg could be calculated. Negative staining shots according to P) showed hollow spherical particles with an outer diameter of 15 nm and an inner diameter of 5 nm.

T) To check the quarternary structure of the pure lumazine synthase a native gel electrophoresis using a 3.5% poly acryl amide gel was carried out. The gel was prepared using 5.7 ml acrylamide stock solution (38.8% (w/v) acrylamide; 1.2% (w/v) N,N′-methylenbisacrylamide), 46 ml gel buffer (0.2 M Na-phosphate, pH 7.2), 13 ml H₂O_{bidest}, 300 μl ammoium peroxodisulfate solution (10% (w/v) in water), 65 μl TEMED (N,N,N′,N′-tetramethylethylene diamine) and 5 mg bromo phenole blue were mixed and applied to a gel preparing device (Pharmacia Biotech, Freiburg, Germany) which contained a GelBond® PAG Film (FMC Bioproducts, Rockland, Me., USA) and polymerized overnight at room temperature. 20 μl of the pure protein solution (concentration: 0.2-1 mg/ml) were applied to the gel. The electrophoresis was carried out in gel buffer at constant 100 mA under temperature control (10° C.). The staining was carried out according to M). The purified recombinant lumazine synthase was observed as a distinct single band on the gel and the behaviour was comparable to a lumazine sample which had been isolated from a wild type Bacillus subtilis strain.

U) To check the quarternary structure respectively the structural homogenity of the pure protein a sedimentation analysis on an analytical ultra centrifuge (Optima XLA with rotor AN60 Ti, Beckman Instruments, Munich, Germany) was carried out (Laue et al., 1992). The protein was centrifuged at 45000 rpm and every 5 min the radial change in the absorption (280 nm) was measured and the movement of the protein determined. The recombinant lumazine synthase sedimented as a single homogenous band, i.e. there was only one molecular species present in the analyzed sample. A sedimentation constant S_{20,w of} 26.3 S could be calculated.

V) For a precise determination of the native molecular weight of the pure lumazine synthase an analytical ultracentrifugation was carried out using an equilibrium sedimentation protocol. For the radius related determination of the protein concentration the absorption was measured at 280 μm. Samples with an absorption of 0.3 at 280 nm were used. 150 μl of this protein solution was filled into the sample sector of a centrifuge cell and 15 μl oil (Fluorochemical FC 43, Beckman, Munich, Germany) was added. The reference sector was filled with 200 μl buffer A (R). The centrifugation was carried out at 3000 rpm til an equilibrium state was reached. The data were calculated using the software XLA-Data-Analysis from Beckman Instruments. The partial specific volume of the protein was estimated according to Cohn and Edsall (1943) based on the partial specific volumes of each amino acid residue of the protein and temperatur corrections. The purified recombinant lumazine synthase showed a molecular weight of 925 kDa at 4° C. (60 mer).

### Example 2

Homologous Expression of the Gene (ribH) Coding for the Lumazine Synthase from Bacillus subtilis in Bacillus subtilis BR151-pBL1 Cells

In comparision to the heterologous expression of the ribH gene in Escherichia coli (Example 1) the homologous expression of the ribH gene in Bacillus subtilis is more efficient relating to the yield of the expressed recombinant protein.

A) Analogous Example 1 A) to E), excepting that oligonucletide EcoRI-RBS-2 (5′ ata ata gaa ttc att aaa gag gag aaa tta act atg 3′) was used instead oligonucleotide EcoRI-RBS-1.

B) The expression vector p602/-CAT was cut analogous to Example 1 F). The resulting DNA-Fragment, with a length of 5269 bp, was purified according to Example 1 B) and used in a ligation protocol.

C) The ligation protocol was carried out analogous to Example 1 G) yielding the expression plasmid p602-BS-LuSy.

D) The transformation of Escherichia coli XL1-celles was carried out analogous to Example 1H), excepting that LB-KAN-agar plates (21 g/l agar; 10 g/l peptone; 5 g/l yeast extrakt; 5 g/l NaCl; 15 mg/l kanamycine) were used instead of LB-AMP-agar plates (the vector p602/-CAT includes a kanamycine resistence gene).

E) The isolation of the resulting expression plasmid was carried out analogous to Example 1 I), excepting that LB-KAN liquid medium (10 g/l peptone; 5 g/l yeast extrakt; 5 g/l NaCl; 15 mg/l kanamycine) was used instead of LB-AMP liquid medium.

F) DNA-sequencing was carried out analogous to Example 1 J), excepting that oligonucleotide Seq-2 (5′ gta taa tag att caa att gtg age gg 3′) was used instead of oligonucleotide Seq-1.

G) The fermentation of the expression strain was carried out analogous to Example 1 K), excepting that LB-KAN liquid medium was used.

H) Cell lysis was carried out analogous to Example 1 L).

I) The SDS-PAGE (protocol analogous Example 1 M)) of the crude lysate of the Escherichia coli expression strain XL1-p602-BS-LuSy showed a distinct overexpressed protein band at a molecular weight of circa 16 kDa. The expression rate of the recombinant lumazine synthase was estimated to 30% related to the total soluble cell proteins of the recombinant strain.

J) The enzymatic activity was measured according to Example 1 N), the protein concentration was determined according to Example 1 O). Using these data a specific activity of circa 3700 U/mg could be calculated in the crude lysate.

K) The preparation of electrocompetent Bacillus subtilis-cells was carried out using a modified protocol according to Brigidi et al. (1989). 500 ml LB-ERY-liquid medium (10 g/l peptone; 5 g/l yeast extract; 5 g/l NaCl; 15 mg/l erythromycine) was inoculated with 5 ml of a BR151[pBL1] cell suspension which was grown overnight at 32° C. The cell culture was then incubated in a incubator at 32° C. At an optical density (578 nm) of 0.6 the culture was placed on ice for 30 min. Cells were harvested by centrifugation (Sorvall-GS-3-Rotor, 2300 rpm, 4° C., 15 min). The cell pellet was suspended in 300 ml 1 mM HEPES buffer (1 mM (N-[2-hydroxyethyl]piperazine-N′-[2-ethanesulfonic acid] in water, pH 7.0) and the mixture was centrifuged again using the same conditions. The resulting pellet was washed twice with 200 ml PEB buffer (272 mM succrose; 1 mM MgCl₂; 7 mM K-phosphate, pH 7.4) and centrufuged again using the same conditions. After the last centrifugation step the pellet was suspended in 16 ml PEB buffer and placed on ice (electrocompetent cells).The electroporation tube (0.4 cm) and the tube holder was cooled on ice for 15 min. 800 μl of electrocompetent cells were mixed with 500-1500 ng of plasmid-DNA (p602-BS-LuSy from E)) in a precooled cap and incubated on ice for 10 min. After transferance into the precooled electroporation tube the electroporation was carried out in a electroporation device from Biorad (Munich, Germany). Conditions: 25 μF, 2.5 kV. After the pulse the suspension was mixed with 6 ml LB-ERY-medium and incubated at 32° C. for 2 h (transformation mixture A). Subsequently 25 ml of LB-ERY-KAN-medium (10 g/l peptone; 5 g/l yeast extract; 5 g/l NaCl; 15 mg/l erythromycine; 15 mg/l kanamycine) were mixed with 1 ml of the transformation mixture A and incubated for 4-8 h in a shaker at 32° C. (transformation mixture B). After this step 20 μl and 200 μl aliquots were removed from transformation mixture B after 2, 4, 6 and 8 h, plated on LB-ERY-KAN-Agar-plates (21 g/l Agar; 10 g/l peptone; 5 g/l yeast extract; 5 μl NaCl; 15 mg/l erythromycine; 15 mg/ml kanamycine) and incubated overnight at 32° C. resulting in the expression strain BR151-pBL1-p602-BS-LuSy. In parallel to that 100 μl, 200 μl and 400 μl of transformation mixture A were plated on LB-ERY-KAN-Agar-plates and incubated overnight at 32° C.

L) The resulting transformants were checked for the presence of the plasmid p602-BS-LuSy using PCR. The PCR was carried out analogous to Example 1 A), excepting that the PCR mixture was prepared without adding template DNA. Aliquots of the PCR mixture were inoculated with cells from fresh transformants using sterile toothpicks yielding a fragment with 498 bp. After this step LB-ERY-KAN-agar plates were inoculated with the specific toothpick (copy of the checked clone) and incubated at 32° C. overnight.

M) The fermentation of the cells was carried out using LB-ERY-KAN-liquid medium (10 g/l peptone; 5 g/l yeast extract; 5 g/l NaCl; 15 mg/l erythromycine; 15 mg/l kanamycine) analogous to Example 1 K), excepting that a temperature of 32° C. instead of 37° C. was used. The cells were incubated for additional 18 h after induction (addition of IPTG).

N) The cells were lysed analogous to Example 1 L).

O) The SDS-PAGE (protocol analogous Example 1 M)) of the crude lysate of the Bacillus subtilis BR151-pBL1-p602-BS-LuSy strain showed a distinct overexpressed protein band at a molecular weight of circa 16 kDa. The expression rate of the recombinant lumazine synthase was estimated to 40-50% related to the total soluble cell proteins.

P) The enzymatic activity was measured according to Example 1 N), the protein concentration was determined according to Example 1 O). Using these data a specific activity of circa 4000 U/mg could be calculated in the crude lysate of the expression strain BR151-pBL1-p602-BS-LuSy.

Q) Negative staining experiments were carried out analogous to Example 1 P) yielding comparable results.

R) The western blot analysis was carried out analogous to Example 1 Q) yielding comparable results.

S) The recombinant lumazine synthase from the Bacillus subtilis strain BR151-pBL1-p602-BS-LuSy could be isolated in pure form using one single column (Sepharose-6B, 2×180 cm, Pharmacia Biotech, Freiburg, Germany). The fermentation was carried out analogous to M) excepting that 1 liter medium was used. The cells were lysed analogous to Example 1 R) in 32 ml lysis buffer, excepting that 30 mg lysozyme was added to the lysis buffer. In a first step the suspension was incubated at 37° C. for 1 h. In a second step the cells were lysed using a ultrasonic device and centrifuged analogous to Example 1 R). Subsequent the supernatant was filtrated (0.22 μm).). The filtrated protein solution was applied to the gel filtration column which had been equilibrated with buffer A analogous Example 1 R). The fractions were checked for lumazine synthase activity according to N). Enzymatic active fractions were collected and concentrated using an ultra centrifuge (Beckman LE 70 with rotor 70Ti; 32000 rpm, 18 h, 4° C.). The purity check was carried out according to Example 1 M) (SDS-PAGE) whereby just one band could be observed at a molecular weight of 16 kDa. The enzymatic activity was measured according to Example 1 N), the protein concentration was determined according to Example 1 O). Using these data a specific activity of 12400 U/mg could be calculated. Negative staining shots according to Example 1 P) showed hollow spherical particles with an outer diameter of 15 nm and an inner diameter of 5 nm.

T) To check the quarternary structure of the isolated pure lumazine synthase experiments analogous to Example 1 S) to U) were carried out yielding comparable results.

### Example 3

Replacement of Cysteine 93 with Serine in the Lumazine Synthase from Bacillus subtilis Using Site Directed Mutagenesis

A) The gene coding for the lumazine synthase from Bacillus subtilis was amplified using the oligonucleotides PNCO-M2 (5′ aga tat ttt cat taa aga gga gaa 3′) as forward primer, which is at his 3′-end identical to ribosome binding site of the vector and which is deleting the vector based EcoRI site at his 5′-end. As reverse primer the oligonucleotide RibH-3 (5′ tat tat gga tcc tta ttc aaa tga gcg gtt taa att tg 3′) was used, which is at his 3′-end identical to the 3′-end of the ribH gene and which introduces a recognition site for the endonuclease BamHI (G*GATCC) directly after the stop codon. The plasmid pNCO-BS-LuSy (Example 1) was used as template for the PCR (Mullis et al., 1986).

10 μl PCR-buffer (75 mM Tris/HCl, pH 9.0; 20 mM (NH₄)₂SO₄; 0.01% (w/v) Tween 20)

6 μl Mg²⁺[1.5 mM]

8 μl dNTP's [je 200 μM]

1 μl PNCO-M2 [0.5 μM]

1 μl RibH-3 [0.5 μM]

1 μl pNCO-BS-LuSy [10 ng]

1 μl Goldstar-Taq-Polymerase [0.5 U] (Eurogentec, Seraing, Belgien)

72 μl H₂O_{bidest}

PCR cycle protocol (GeneAmp® PCR System 2400; Perkin Elmer):

1. 5.0 min 95° C.

2. 0.5 min 94° C.

3. 0.5 min 50° C.

4. 0.5 min 72° C.

5. 7.0 min 72° C.

6. ∞ 4° C.

Steps 2.-4. were repeated 20 times.

B) The PCR mixture was analysed and purified analogous to Example 1 B) yielding a DNA fragment with a length of 505 bp.

C) A part of the ribH gene coding for the lumazine synthase from Bacillus subtilis was amplified using the oligonucleotides PNCO-M1(5′ gtg agc gga taa caa ttt cac aca g 3′) as forward primer, which anneals to the vector sequence in 5′ direction of the EcoRI site at position 88, and C93S (5′ gca gct tca ttc gaa aca taa tcg taa tg 3′), which is responsible for the replacement of the amino acid residue cysteine 93 by serine via site directed mutagenesis and which is introducing a new site for the restriction endo nuclease BstBI for the detection of the mutation. The plasmid pNCO-BS-LuSy (Example 1) was used as template for the PCR (Mullis et al., 1986). The PCR protocol, the analysis and the purification of the PCR mixture was carried out analogous to A) and B) yielding a DNA fragment with a length of 256 bp.

D) Extension of the DNA-fragment (256 bp, containing the mutation and an intact EcoRI site at the 5′ end) from C) via combination with the DNA-fragment (505 bp, representing the total ribH, but with a deleted EcoRI site at the 5′ end) from B) and PCR. Equimolar amounts (each 500 fmol) of the DNA fragments from B) and C) served as primers in the PCR.

10 μl buffer

6 μl Mg²⁺[1.5 mM]

8 μl dNTP's [each 200 μM]

1 μl DNA-fragment from B) (500 fmol)

1 μl DNA-Fragment from C) (500 fmol)

1 μl Goldstar-Taq-polymerase [0.5 U]

73 μl H₂O_{bidest}

PCR cycle protocol (GeneAmp® PCR System 2400; Perkin Elmer):

1. 5.0 min 95° C.

2. 0.5 min 94° C.

3. 0.5 min 65° C.

4. 0.5 min 72° C.

5. 7.0 min 72° C.

6. ∞ 4° C.

Steps 2.-4. were repeated 20 times.

E) An aliquot of the PCR mixture from D) served as template for a PCR using the oligonucletides PNCO-M1/RibH-3 as forward and as reverse primers. The PCR was carried out analogous A), excepting that the steps 2.-4. were repeated 25 times.

F) The PCR mixture was analysed and purified analogous Example 1 B), yielding a DNA-fragment with 528 bp.

G) The further handling was carried out analogous Example 1 E)-I). The presence of the mutation was checked via digestion of the isolated plasmid pNCO-BS-LuSy-C93S with the restriction endonuclease BstBI (TT*CGAA) yielding DNA fragments with 3698 bp and 181 bp.

H) The DNA sequencing was carried out analogous to Example 1 J).

I) The isolation of the protein and the quality checks were carried out analogous to Example 1 K)-S) yielding compareable results, meaning that there were no significant differences to wild type lumazine synthase.

### Example 4

Replacement of Cysteine 139 with Serine in the Lumazine Synthase from Bacillus subtilis Using Site Directed Mutagenesis

A) The gene coding for the lumazine synthase from Bacillus subtilis was amplified analogous Example 3 A) using the oligonucleotides PNCO-M2 and RibH-3 as primers and the plasmid pNCO-BS-LuSy (Example 1) as template and purified analogous Example 1 B) yielding a DNA fragment with 505 bp.

B) A part of the ribH gene coding for the lumazine synthase from Bacillus subtilis was amplified using the oligonucleotides PNCO-M1(5′ gtg agc gga taa caa ttt cac aca g 3′) as forward primer, which anneals to the vector sequence in 5′ direction of the EcoRI site, and C139S (5′ ggc aga aac agc tga atc tac acc ttt gtt g 3′), which is responsible for the replacement of the amino acid residue cysteine 139 by serine via site directed mutagenesis and which is introducing a new site for the restriction endo nuclease PvuII for the detection of the mutation. The plasmid pNCO-BS-LuSy (Example 1) was used as template for the PCR (Mullis et al., 1986). The PCR protocol, the analysis and the purification of the PCR mixture was carried out analogous to Example 3 A) and Example 1 B) yielding a DNA fragment with a length of 394 bp.

C) The further handling was carried out analogous to Example 3 D) —H). The mutation was checked via digestion of the plasmid pNCO-BS-LuSy-C139S with the restriction endonuclease PvuII (CAG*CTG) yielding DNA fragments with 3539 bp and 340 bp.

D) The isolation of the protein and the quality checks were carried out analogous to Example 1 K) to S) yielding compareable results, meaning that there were no significant differences to wild type lumazine synthase.

### Example 5

Replacement of Cysteine 93 and 139 with Serine in the Lumazine Synthase from Bacillus subtilis Using Site Directed Mutagenesis

A) The construction of the double mutant plasmid pNCO-BS-LuSy-C93/139S was carried out analogous Example 4, excepting that the plasmid pNCO-BS-LuSy-C93S was used as template for the PCR.

B) The isolation of the protein and the quality checks were carried out analogous to Example 1 K)-S) yielding compareable results, meaning that there were no significant differences to wild type lumazine synthase.

Construction of Expression Vectors for the Fusion of Proteins to the N- and to the C-Terminus of the Lumazine Synthase from Bacillus subtilis

The following examples describe the preparation of Escherichia coli expression vectors for the fusion of genes or synthetic DNA fragments to the 5′- or the 3′-end of the ribH gene coding for the lumazine synthase from Bacillus subtilis. According to the invitation the plasmid contains the following prefered vector elements: A promotor sequence from the bacteriophage T5, an operator sequence from the lac-operon from Escherichia coli, an ampicilline resistance marker gene and an Escherichia coli plasmid origin of replication.

### Example 6

Vector for the Fusion of DNA Coding for a Target Peptide to the 5′-End of the ribH Gene (Coding for the Lumazine Synthase) from Bacillus subtilis

A) The gene coding for the lumazine synthase from Bacillus subtilis was amplified analogous Example 1 A), excepting that the oligonucleotide N1 (5′ act atg gcg gcg gcg cgt agc tgc gcg gcc gct atg aat atc ata caa gga aat tta g 3′), which introduced a recognition site for the restriction endonuclease NotI (GC*GGCCGC) in close contact to the start codon of the ribH gene, was used as forward primer and the oligonucleotide RibH-4 (3′ tat tat gga tcc aaa tta ttc aaa tga gcg gtt taa att tg 3′) which introduced a recognition site for the endonuclease BamHI (G*GATCC) in close distance to the stop codon, was used as reverse primer. The plasmid pRF2 (Example 1) was used as template for the PCR.

B) The PCR mixture was analyzed and purified analogous to Example 1 B) yielding a DNA-fragment with 513 bp.

C) 10 ng of the isolated DNA fragment from B) served as a template for a second PCR using the oligonucleotide N2 (5′ ata ata gaa ttc att aaa gag gag aaa tta act atg gcg gcg gcg cgt agc tgc 3′), which extended the DNA fragment from B) in 5′ direction whereby a ribosome binding site and a recognition site for the restriction endonuclease EcoRI (G*AATTC) was introduced as forward primer and the oligonucleotide RibH-4 as reverse primers.

D) The further handling was carried out analogous to Example 1 B), E)-J) yielding the plasmid pNCO-N-BS-LuSy.

### Example 7

Vector for the Fusion of DNA Coding for a Target Peptide to the 3′-end of the ribH Gene (Coding for the Lumazine Synthase) from Bacillus subtilis

A) The gene coding for the lumazine synthase from Bacillus subtilis was amplified analogous to Example 1 A), excepting that the oligonucleotide EcoRI-RBS-2 (Example 2 A)) was used as forward primer and oligonucleotide C2 (5′ ttt tcg gga tcc ttt taa act gtt tgc ggc cgc taa ttc aaa tga gcg gtt taa att tg 3′), which introduced a new site for the restriction endonuclease NotI in close contact to the last coding base triplett of the ribH gene and which introduced a new recognition site for the restriction endonuclease BamHI in a distance of 13 nucleotides downstream to the NotI site, was used as reverse primer and the plasmid pNCO-BS-LuSy (Example 1) was used as template for the PCR. The stop codon of the wild type ribH gene was replaced by the base triplett TTA coding for the amino acid residue leucine.

B) The further handling was carried out analogous to Example 1 B), E)-J) yielding the plasmid pNCO-C-BS-LuSy.

Fusion of Complete ORFs (Open Reading Frames) to the N-Terminus or to the C-Terminus of the Lumazine Synthase from Bacillus subtilis

The following examples describe the fusion of complete genes to the 5′- or the 3′-end of the ribH gene coding for the lumazine synthase from Bacillus subtilis. These examples illustrate the feasibility to fuse complete, biological active target proteins to the N-terminus or to the C-terminus of the icosahedral lumazine synthase from Bacillus subtilis.

### Example 8

Fusion of the Dihydrofolate Reductase (folA; DHFR) from Escherichia coli to the N-Terminus of the Lumazine Synthase (ribH) from Bacillus subtilis

A) The gene coding for the dihydrofolate reductase (DHFR) from Escherichia coli was amplified analogous to Example 1 A), excepting that the oligonucleotide EC-DHFR-1 (5′ gag gag aaa tta act atg atc agt ctg att gcg g 3′), which bound at its 3′-end to the 5′-end of the folA gene and which introduced a part of an optimized ribosome binding site upstream to the start codon, was used as forward primer and the oligonucleotide EC-DHFR-2 (5′ cta gcc gta aat tct ata gcg gcc gca cgc cgc tcc aga atc 3′), which bound at its 3′-end to the 3′-end of the DHFR gene and which introduced a new recognition site for the restriction endonuclease NotI directly after the last coding base triplett of the folA gene, was used as reverse primer. Circa 50 ng of isolated chromosomal Escherichia coli DNA (RR28) were used as template for the PCR.

B) The PCR mixture was analyzed and purified analogous to Example 1 B) yielding a DNA-fragment with 513 bp.

C) A second PCR was carried out analogous to Example 1 C), excepting that the oligonucleotide BS-MfeI (5′ ata ata caa ttg att aaa gag gag aaa tta act atg 3′), which extended the ribosome binding site in 5′-direction and which introduced a site for the restriction endonuclease MfeI (C*AATTG) was used as forward primer and the oligonucleotide EC-DHFR-2 was used as reverse primer.

D) The PCR mixture was analyzed and purified analogous to Example 1 B) yielding a DNA-fragment with 531 bp.

E) The isolated DNA fragment from D) was digested using the restriction endonuclease MfeI (the DNA-overhang generated by Mfel (C*AATTG) is compatible with the DNA-overhang generated by EcoRI (G*AATTC)).

30.0 μl DNA fragment from D)

5.0 μl MfeI [50 U]

10.0 μl buffer 4 (10×; 50 mM K-acetate, 20 mM tris-acetate, 10 mM Mg-acetate, 1 mM dithiothreitol, pH 7.9) 55.0 μl H₂O_{bidest}

The enzymes were purchased from New England Biolabs (Schwalbach, Germany). The mixture was incubated for 150 min at 37° C. After incubation the mixture was purified as described under Example 1 B) and used for the digestion with the restriction endonuclease NotI.

F) In a second step the purified DNA fragment from E) was digested with the restriction endonuclease NotI.

30.0 μl DNA fragment from E)

5.0 μl NotI [50 U]

10.0 μl buffer 3 (10×; 100 mM NaCl, 50 mM Tris-HCl, 10 mM MgCl₂, 1 mM dithiothreitol, pH 7.9)

55.0 μl H₂O_{bidest}

The enzymes were purchased from New England Biolabs (Schwalbach, Germany). The mixture was incubated for 150 min at 37° C. After incubation the mixture was purified as described under Example 1 B) and used in a ligation protocol.

G) In a first step 5 μg of the expression vector pNCO-N-BS-LuSy in a volume of 30 μl were digested with the restriction endonuclease NotI analogous to F) and purified analogous to Example 1 B).

H) In a second step the DNA fragment from G) was digested with the restriction endonuclease EcoRI.

30.0 μl vector-fragment from G)

2.5 μl EcoRI [62,5 U]

20.0 μl OPAU (10×; 500 mM K-acetate, 100 mM Mg-acetate, 100 mM Tris-acetate, pH 7.5)

47.5 μl H₂O_{bidest}

The enzymes were purchased from New England Biolabs (Schwalbach, Germany). The mixture was incubated for 150 min at 37° C. After incubation the mixture was purified as described under Example 1 B) yielding a fragment with a length of 3863 bp and used for in a ligation protocol.

I) The further handling was carried out analogous to Example 1 G)-L) yielding the plasmid pNCO-EC-DHFR-BS-LuSy.

J) The SDS-PAGE was carried out analogous to Example 1 M). In the crude lysate of the strain XL1-pNCO-EC-DHFR-BS-LuSy an overexpressed protein band with a molecular weight of circa 34.5 kDa could be observed, which was not detectable in a strain without the plasmid pNCO-EC-DHFR-BS-LuSy. The expression rate of this protein could be estimated to 40-50% (concerning to the total soluble cell proteins).

K) The enzymatic activity was measured according to Example 1 N), the protein concentration was determined according to Example 1 O). Using these data a specific activity of circa 3700 U/mg could be calculated in the crude lysate which was campareable with recombinant wild type lumazine synthase.

L) The isolation of the fusion protein was carried out analogous to Example 2 S), excepting that the centrifugation of the protein in an ultra centrifuge was carried out at 28000 rpm. Negative staining experiments were carried out analogous to Example 1 P), excepting that the pictures showed hollow spherical particles with an outer diameter of circa 20 nm and an inner diameter of circa 5 nm.

M) To check the quarternary structure of the isolated pure lumazine synthase an experiment analogous to Example 1 S) was carried out. It could be observed that the fusion protein (EC-DHFR-BS-LuSy), based on the increased diameter of the particle, migrates slower on the native gel than the wild type lumazine synthase.

### Example 9

Fusion of the Maltose Binding Protein (malE; MBP) from Escherichia coli to the N-Terminus of the Lumazine Synthase (ribH) from Bacillus subtilis

A) The gene coding for the maltose binding protein from Escherichia coli was amplified analogous Example 1 A), excepting that the oligonucleotide MALE-1 (5′ gag gag aaa tta act atg aaa atc gaa gaa ggt aaa c 3′), which bound at its 3′-end to the 5′-end of the MBP gene and which introduced a part of an optimized ribosome binding site upstream to the start codon, was used as forward primer and oligonucleotide MALE-2 (5′ gca ggt cga ctc tag cgg ccg cga att ctg 3′), which bound at its 3′-end to the 3′-end of the MBP gene and which introduced a new recognition site for the restriction endonuclease NotI nearby the 5′-region of the MBP gene, was used as reverse primer. Circa 10 ng of the plasmid pMAL-C2 (New England Biolabs, Schwalbach, Germany) were used as template for the PCR.

B) The PCR mixture was analyzed and purified analogous to Example 1 B) yielding a DNA-fragment with 1210 bp.

C) A second PCR was carried out analogous to Example 1 C), excepting that the oligonucleotide BS-MfeI (5′ ata ata caa ttg att aaa gag gag aaa tta act atg 3′), which extended the ribosome binding site in 5′-direction and which introduced a recognition site for the restriction endonuclease MfeI was used as forward primer and the oligonucleotide MALE-2 was used as reverse primer.

D) The PCR mixture was analyzed and purified analogous to Example 1 B) yielding a DNA-fragment with 1227 bp.

E) The further handling was carried out analogous to Example 8 E)-I) yielding the plasmid pNCO-EC-MBP-BS-LuSy.

F) The SDS-PAGE was carried out analogous to Example 1 M). In the crude lysate of the strain XL1-pNCO-EC-MBP-BS-LuSy an overexpressed protein band with a molecular weight of circa 59.5 kDa could be observed, which was not detectable in a strain without the plasmid pNCO-EC-MBP-BS-LuSy. The expression rate of this protein could be estimated to 40-50% (related to the total soluble cell proteins).

G) The enzymatic activity was measured according to Example 1 N), the protein concentration was determined according to Example 1 O). Using these data a specific activity of circa 3700 U/mg could be calculated in the crude lysate which was comparable with recombinant wild type lumazine synthase.

H) The isolation of the fusion protein was carried out analogous to Example 2 S), excepting that the centrifugation of the protein in a ultra centrifuge was carried out at 28000 rpm. Negative staining experiments were carried out analogous to Example 1 P), excepting that the pictures showed hollow spherical particles with an outer diameter of circa 25 nm and an inner diameter of circa 5 nm.

I) To check the quarternary structure of the isolated pure lumazine synthase an experiment analogous to Example 1 S) was carried out. It could be observed that the fusion protein (EC-MBP-BS-LuSy)—based on the increased diameter of the particle—migrates slower on the native gel than EC-DHFR-BS-LuSy or the wild type lumazine synthase.

### Example 10

Fusion of the Dihydrofolate Reductase (folA, DHFR) from Escherichia coli to the C-Terminus of the Lumazine Synthase (ribH) from Bacillus subtilis

A) The gene coding for the dihydrofolate reductase (DHFR) from Escherichia coli was amplified analogous to Example 1 A), excepting that the oligonucleotide EC-FolA-1 (5′ ata gtg gcg aca atg cgg ccg ctg gtg gag gcg gaa tga tca gtc tga ttg cgg cg 3′), which bound at its 3′-end to the 5′-end of the DHFR gene and which introduced upstream to the start codon of the DHFR gene a site for the restriction endonuclease NotI, was used as forward primer and oligonucleotide EC-FolA-2 (5′ ttc tat gga tcc tta ccg ceg ctc cag aat c 3′), which bound at its 3′-end to the 3′-end of the DHFR gene and which introduced a site for the restriction endonuclease BamHI directly after the stop codon of the DHFR gene, was used as reverse primer. Circa 50 ng of isolated chromosomal Escherichia coli DNA (RR28) were used as template for the PCR.

B) The PCR mixture was analyzed and purified analogous to Example 1 B) yielding a DNA-fragment with 524 bp.

C) The isolated DNA fragment from B) was digested using the restriction endonuclease BamHI.

30.0 μl DNA fragment from B)

3.0 μl BamHI [60 U]

20.0 μl OPAU (10×)

47.0 μl H₂O_{bidest}

The enzymes were purchased from Pharmacia Biotech (Freiburg, Germany). The mixture was incubated for 150 min at 37° C. After the incubation the mixture was purified as described under Example 1 B) and used for the digestion with the restriction endonuclease NotI.

D) In a second step the purified DNA fragment from C) was digested with the restriction endonuclease NotI analogous to Example 5 F). After the incubation the mixture was purified as described under Example 1 B) and used for in a ligation protocol.

E) In a first step 5 μg of the expression vector pNCO-C-BS-LuSy (Example 4) in a volume of 30 μl were digested with the restriction endonuclease NotI and BamHI analogous to C) and D). After the incubation the mixture was purified as described under Example 1 B) yielding a fragment with a length of 3880 bp and used for in a ligation protocol.

F) The further handling was carried out analogous to Example 1 G)-L) yielding the plasmid pNCO-BS-LuSy-EC-DHFR.

G) The SDS-PAGE was carried out analogous to Example 1 M). In the crude lysate of the strain XL1-pNCO-BS-LuSy-EC-DHFR an overexpressed protein band with a molecular weight of circa 34.8 kDa could be observed, which was not detectable in a strain without the plasmid pNCO-BS-LuSy-EC-DHFR. The expression rate of this protein could be estimated to 25% (related to the total soluble cell proteins).

H) The enzymatic activity was measured according to Example 1 N), the protein concentration was determined according to Example 1 O). Using these data a specific activity of circa 3700 U/mg could be calculated in the crude lysate which was campareable with recombinant wild type lumazine synthase.

I) The isolation of the fusion protein was carried out analogous to Example 2 S), excepting that the centrifugation of the protein in a ultracentrifuge was carried out at 28000 rpm. Negative staining experiments were carried out analogous to Example 1 P), excepting that the pictures showed hollow spherical particles with an outer diameter of circa 20 nm and an inner diameter of circa 5 nm.

J) To check the quarternary structure of the isolated pure lumazine synthase an experiment analogous to Example 1 S) was carried out. It could be observed that the fusion protein (BS-LuSy-EC-DHFR)—based on the increased diameter of the particle—migrated slower on the native gel than the wild type lumazine synthase.

Linking of an Epitop (17 Aminoacid Residues) of the VP2 Surface Protein from a Mammal Virus to the N-Terminus, to the C-Terminus and to Both Termini of the Lumazine Synthase from Bacillus subtilis

The following examples describe the fusion of short peptides to eather the N-terminus or the C-terminus or both termini of the icosahedral lumazine synthase from Bacillus subtilis under formation of hollow spherical particles consisting of 60 subunits. Based on the fusion to both termini 120 epitops could be presented on the surface of the lumazine synthase.

The peptide with a length of 17 aa is a highly conserved part of the VP2 surface protein from different mammal viruses, e.g. ‘mink enteritis virus’, ‘feline panleukopenia virus’, ‘canine parvo virus’.

### Example 11

Fusion of the VP2 Epitop to the N-Terminus of the Lumazine Synthase (ribH) from Bacillus subtilis

A) The gene coding for the lumazine synthase from Bacillus subtilis was amplified analogous to Example 1 A), excepting that oligonucleotide N-VP2-1 (5′ ggt cag ccg gct gtt cgt aac gaa cgt atg aat atc ata caa gga aat tta gtt ggt ac 3′), which bound at its 3′-end to the 5′-end of the ribH gene and which coded for a part of the VP2 epitop at the 5′-end, was used as forward primer and oligonucleotide RibH-3 (Example 3) was used as reverse primer. The plasmid pRF2 served as template for the PCR.

B) The PCR mixture was analyzed and purified analogous to Example 1 B) yielding a DNA-fragment with 504 bp and served as template for a second PCR step.

C) 10 ng of the isolated DNA fragment from B) served as a template for a second PCR using the oligonucleotide N-VP2-2 (5′ gag gag aaa tta act atg ggg gac ggt gct gtt cag ccg gac ggt ggt cag ccg gct gtt cgt aac gaa cg 3′), which extended the DNA coding for the VP2 epitop from B) in 5′ direction and which introduced a part of a ribosome binding site, as forward primer and oligonucleotide RibH-3 as reverse primer.

D) The PCR mixture was analyzed and purified analogous to Example 1 B) yielding a DNA-fragment with 549 bp and served as template for a third PCR step.

E) The third PCR step was carried out analogous to Example 1 C), excepting that the oligonucleotides EcoRI-RBS-2 (Example 2 A) and RibH-3 were used as forward and as reverse primers.

F) The PCR mixture was analyzed and purified analogous Example 1 B), yielding a DNA-fragment with 567 bp.

G) The further handling was carried out analogous to Example 1 E)-L) yielding the Escherichia coli expression strain XL1-pNCO-N-VP2-BS-LuSy.

H) The SDS-PAGE was carried out analogous to Example 1 M). In the crude lysate of the strain XL1-pNCO-N-VP2-BS-LuSy an overexpressed protein band with a molecular weight of circa 18.2 kDa could be observed which was not detectable in a strain without the plasmid pNCO-N-VP2-BS-LuSy. The expression rate of this protein could be estimated to 10% (related to the total soluble cell proteins).

I) The enzymatic activity was measured according to Example 1 N), the protein concentration was determined according to Example 1 O). Using these data a specific activity of circa 3700 U/mg could be calculated in the crude lysate which was campareable with recombinant wild type lumazine synthase.

### Example 12

Fusion of the VP2 Epitop to the C-Terminus of the Lumazine Synthase (ribH) from Bacillus subtilis

A) The gene coding for the lumazine synthase from Bacillus subtilis was amplified analogous to Example 1 A), excepting that the oligonucleotide C-VP2-1 (5‘cca ccg tcc ’ ggc tga aca gca ccg tca cct tcg aaa gaa cgg ttt aag ttt gcc 3′), which bound at its 3′-end to the 3′-end of the ribH gene and which introduced—directly after the last coding base triplett of the ribH gene—a part of the DNA coding for the VP2 epitop at its 5′-end, was used as reverse primer. The plasmid pRF2 served as template for the PCR.

B) The PCR mixture was analyzed and purified analogous Example 1 B), yielding a DNA-fragment with 506 bp, which served as template for a second PCR step.

C) A second PCR was carried out analogous to Example 1 C), excepting that the oligonucleotide EcoRI-RBS-2 (Example 2 A)) was used as forward primer and oligonucleotide C-VP2-2 (5′ ata tat gga tcc taa cgt tcg tta cga aca gcc ggc tga cca ccg tcc ggc tga aca gca ccg tc 3′), which extended the DNA coding for the VP2 epitop in 3′-direction and which introduced a stop codon after the last coding base triplett of the VP2 epitop and a recognition site for the restriction endonuclease BamHI (G*GATCC), was used as reverse primer.

D) The PCR mixture was analyzed and purified analogous Example 1 B), yielding a DNA-fragment with 564 bp.

E) The further handling was carried out analogous to Example 1 E)-L) yielding the Escherichia coli expression strain XL1-pNCO-C-VP2-BS-LuSy.

F) The SDS-PAGE was carried out analogous to Example 1 M). In the crude lysate of the strain XL1-pNCO-C-VP2-BS-LuSy an overexpressed protein band with a molecular weight of circa 18.2 kDa could be observed, which was not detectable in a strain without the plasmid pNCO-C-VP2-BS-LuSy. The expression rate of this protein could be estimated to 10% (related to the total soluble cell proteins).

G) The enzymatic activity was measured according to Example 1 N), the protein concentration was determined according to Example 1 O). Using these data a specific activity of circa 3700 U/mg could be calculated in the crude lysate which was comparable with recombinant wild type lumazine synthase.

### Example 13

Fusion of the VP2 Epitop to the N-Terminus and to the C-Terminus of the Lumazine Synthase (ribH) from Bacillus subtilis

A) The expression plasmid pNCO-N-VP2-BS-LuSy (Example 11) served as template for a PCR, which was carried out analogous to Example 12 A)-D).

B) The further handling was carried out analogous to Example 1 E)-L) yielding the Escherichia coli expression strain XL1-pNCO-N/C-VP2-BS-LuSy.

C) The SDS-PAGE was carried out analogous to Example 1 M). In the crude lysate of the strain XL1-pNCO-N/C-VP2-BS-LuSy an overexpressed protein band with a molecular weight of circa 20 kDa could be observed, which was not detectable in a strain without the plasmid pNCO-N/C-VP2-BS-LuSy. The expression rate of this protein could be estimated to 10% (related to the total soluble cell proteins).

D) The enzymatic activity was measured according to Example 1 N), the protein concentration was determined according to Example 1 O). Using these data a specific activity of circa 3700 U/mg could be calculated in the crude lysate which was comparable with recombinant wild type lumazine synthase.

### Example 14

Linking of a Peptide (13 Aminoacid Residues; Bio-Peptide) to the C-Terminus of the Lumazine Synthase (ribH) from Bacillus subtilis

A) The gene coding for the lumazine synthase from Bacillus subtilis was amplified analogous to Example 1 A), excepting that the oligonucleotide EcoRI-RBS-2 (Example 2 A)) was used as forward primer and oligonucleotide C-Biotag-1 (5′ cat agc ttc gaa gat gcc gcc gag tgc ggc cgc ttc gaa aga acg gtt taa gtt tgc cat ttc 3′), which bound at its 3′-end to the 3′-end of the ribH gene and which introduced directly after the last coding base triplett of the ribH gene a DNA fragment coding for three alanines (linker residues) and which introduced in 3′-direction to the DNA sequence coding for the linker residues a DNA fragment coding for a part of the Bio-Peptide, was used as reverse primer. The plasmid pNCO-BS-LuSy (Example 1) served as template for the PCR.

B) The PCR mixture was analyzed and purified analogous Example 1 B) yielding a DNA-fragment with 528 bp and served as template for a second PCR step.

C) The second PCR step was carried out analogous to Example 1 C), excepting that the oligonucleotide EcoRI-RBS-2 (Example 2 A) was used as forward primer and oligonucleotide C-Biotag-2 (5′ tat tat gga tcc tta gcg cca ctc cat ctt cat agc ttc gaa gat gcc gcc gag tgc ggc 3′), which extended the DNA sequence coding for the Bio-peptide in 3′-direction and which introduced a stop codon directly after this coding sequence and which introduced a recognition site for the restriction endonuclease BamHI (G*GATCC), was used as reverse primer.

D) The PCR mixture was analyzed and purified analogous Example 1 B) yielding a DNA-fragment with 558 bp.

E) The further handling was carried out analogous to Example 1 E)-L) yielding the Escherichia coli expression strain XL1-pNCO-C-Biotag-BS-LuSy.

F) The enzymatic activity was measured according to Example 1 N), the protein concentration was determined according to Example 1 O) but no activity based on a recombinant expression of a lumazine synthase could be detected.

G) The SDS-PAGE was carried out analogous to Example 1 M). In the crude lysate of the strain XL1-pNCO-C-Biotag-BS-LuSy no overexpressed protein band with a molecular weight of circa 18.5 kDa could be observed.

H) To check the total expressed cell proteins (total cell extract, soluble an insoluble proteins) of the strain XL1-pNCO-C-Biotag-BS-LuSy cells were fermented and handled analogous to Example 1 K). 1/12 of the resulting cell pellet was suspended in 300 μl sample buffer (Example 1 M)) and incubated on a boiling water bath for 15 min. After cooling down to 4° C. the suspension was centrifuged (15000 rpm, 5 min, 4° C.). 8 μl of the clear supernatant was applied to a SDS-PAGE analogous to Example 1 M). A recombinant protein band with a molecular weight of 18.5 kDa could be observed in the total cell extract of the strain XL1-pNCO-C-Biotag-BS-LuSy but in an insoluble form (inclusion bodies). The observed protein band corresponded to circa 15% of the total cell extract of the strain.

I) To verify that the observed recombinant protein band corresponds to the arteficial lumazine synthase fusion protein (C-Biotag-BS-LuSy) a western blot analysis was carried out analogous to Example 1 Q), excepting that in addition to the soluble cell extract the total cell extract was analyzed. After the development of the PVDF-membrane recombinant lumazine synthase fusion protein could be detected mostly in the total cell but hardly in the soluble cell extracts.

J) Detection of the biotinylation of the fusion protein: Starting from a denaturating SDS-polyacryl amide gel (Example 1 M)) proteins were transfered on a PVDF membrane by electro blotting (current: 40 mA, 2 h). After transferance of the proteins, the membrane was rinsed in antibody-washing-solution-A (Example 1 Q)). Afterwards the membrane was incubated in antibody-washing-solution-B (Example 1 Q)) for 1 h at room temperature. Subsequent the membrane was incubated overnight in 15 ml antibody-washing-solution-C (Example 1 Q)) containing 20 μl streptavidin-alkaline-phosphatase-conjugate (Promega, Madison, Wis., USA). Afterwards the membrane was washed 3 times using each 5 ml antibody-washing-solution-A. The visualization of streptavidin bound to the immobilized biotin was carried out using the substrates for the alkaline phosphatase. 50 μl BCIP-stock solution (25 mg 5-bromo-4-chloro-3-indolyl phosphate (Sigma, Munich, Germany) solved in 500 μl dimethylformamide, store at 4° C. in the dark) and 100 μl NBT-stock solution (50 mg nitro blue tetrazolium (Sigma, Munich, Germany) solved in a mixture of 700 μl dimethylformamide and 300 μl water, store at 4° C. in the dark) were mixted together in 15 ml alkaline phosphatase buffer (100 mM tris-HCl, 100 mM NaCl, 5 mM MgCl₂, pH 9.5). The lumazine synthase with covalently bound biotin could be detected on the membrane as a single blue band with a molecular weight of circa 18.5 kDa. This protein band couldn't be observed in an Escherichia coli strain without the expression plasmid pNCO-C-Biotag-BS-LuSy. The reaction of the alkaline phosphatase was stopped via incubation of the membrane in 5 ml of stop solution (20 mM tris-HCl, 25 mM EDTA-Na₂, pH 8.0).

K) For the refolding of the expressed recombinant fusion protein the soluble protein fraction was removed analogous to example 1 L).

L) The pellet resulting from K) was incubated in refolding buffer A (100 mM K-phosphate, pH 7.0, 6 M urea, 6 mM 5-nitro-6-(D-ribitylamino)2,4-(1H,3H)-pyrimidindione, 100 mM dithiothreitol (DTT)) for 24 h at room temperature. The resulting solution was dialysed twice against the 10-fold volume of refolding buffer B (100 mM K-phosphate, pH 7.0, 1 mM 5-nitro-6-(D-ribitylamino)2,4-(1H,3H)-pyrimidindione, 1 MM DTT) for 12 h at 4° C. The precipitated proteins in the dialysed solution were removed via centrifugation (Sorvall SS34-rotor; 15000 rpm; 20 min; 4° C.). The soluble proteins in the resulting supernatant were concentrated using an ultracentrifuge (Beckman TFT 70-rotor; 32000 rpm; 16 h; 4° C.). The analytic of the proteins was carried out analogous to J), Example 1 S) and Example 1 P) yielding an arteficial protein consisting of 60 subunits forming an icosahedral structure.

M) To check the accessibility of the biotin molecules on the surface of the icosahedron an ELISA protocol (enzyme linked immunosorbent assay) was carried out in microtiter plates 96 wells; 8 wells in a column, 12 wells in a row). 100 μl avidin stock solution (Sigma, Munich, Germany) with a concentration of 1 mg/ml were diluted in 20 ml coating buffer (20 mM Na-carbonate, pH 9.6) yielding the standard solution. The wells of the microtiter plate were filled with 100 μl standard solution and incubated overnight at room temperature. Subsequently the standard solution was removed and each well was washed 3× with 200 μl PBS (20 mM Na-phosphate, 130 mM NaCl, pH 7.2). 350 μl Solution A (3% skimmed milk powder in PBS buffer) were added to each well and incubated for 1 h at 37° C. Afterwards Solution A was removed and 100 μl protein solution from L) (circa 1 mg/ml) was added to the first well of each column of the microtiter plate. 50 μl dilution buffer (1% skimmed milk powder in PBS) was added to the wells 2-8 in the same column. In a subsequent step 50 μl of the protein solution in the first well was removed and added to the solution in well 2 and mixed with the dilution buffer. 50 μl of this diluted protein solution from well 2 was removed and added to the solution in well 3 and mixed. 50 μl from 3 to 4, 50 μl from 4 to 5, 50 μl from 5 to 6, 50 μl from 6 to 7, 50 μl from 7 to 8, 50 μl from 8 to waste (dilution: log 2). The samples were incubated for 2 h at 37° C. Afterwards the solution in the wells were removed totally and the wells were washed 3× with 350 μl PBS. Subsequently 15 μl streptavidin-alkaline-phospatase conjugate (Promega, Madison, Wis., USA) were mixed with 20 ml dilution buffer (detection solution). To each well 100 μl Detection solution was added and the mixture was incubated 1 h at 37° C. Afterwards the solution was removed totally and the wells were washed 3× with 350 μl PBS. For the visualization 150 μl substrate solution (10 mg p-nitrophenyl phosphate (Sigma, Munich, Germany) in 10 ml alkaline phosphatase buffer analogous to J)) were added to each well and incubated at room temperature. The extinction was measured at 405 nm in an ELISA reader. The results showed biotin molecules located on the surface of the lumazine synthase fusion protein. The signal went through an optimum. If the concentration of the lumazine synthase fusion protein was highest a sterical hindrance for the bindung of the streptavidin detection molecules could be observed. If the concentration was decreased, more and more biotin molecules got accessible for the streptavidin molecules and the measured signal got more intensive (going through an opimal concentration).

Labeling of of the C-Terminus of the Lumazine Synthase from Bacillus subtilis with a Reactive Amino Acid Residue

The following examples decribe the fusion of a reactive (for chemical reaction) amino acid residues (lysine and cysteine) to the C-terminus of the lumazine synthase from Bacillus subtilis. There are some lysine residues located on the outer surface of the lumazine but these residues are involved in structural elements of the capsid and not well accessible for chemical reactions. There are no free cysteine residues located on the outer surface of the lumazine synthase which could be used for chemical reaction via the thiole group.

To decrease the sterical hindrance and to increase the accessibility of the reactive groups a linker (tentacle-linker; spacer) was introduced between the reactive amino acid and the C-terminus of the lumazine synthase.

### Example 15

Extension of the C-Terminus of the Lumazine Synthase from Bacillus subtilis and Introduction of a Basic Amino Acid Residue (Lysine) as a Basis for the Chemical Coupling of Target Molecules

A) The gene coding for the lumazine synthase from Bacillus subtilis was amplified analogous to Example 1 A), excepting that the oligonucleotide EcoRI-RBS-2 (Example 2 A)) was used as forward primer and oligonucleotide C-Lys165 (5′ tat tat gga tcc tta ttt acc aga gcc acc acc aga acc acc gcc acc ttc gaa aga acg gtt taa gtt tgc cat ttc 3′), which bound at its 3′-end to the 3′-end of the ribH gene and which introduced in close contact to the last coding base triplett of the ribH gene a DNA sequence coding for the peptide (Gly)₄Ser-(Gly)₃Ser-Gly-Lys was used as reverse primer and the plasmid pNCO-BS-LuSy (Example 1) was used as template for the PCR. Directly after the base triplett coding for the lysine residue (aaa) at position 165 in the arteficial protein, a stop codon and a recognition site for the restriction endonuclease BamHI was introduced.

B) The PCR mixture was analyzed and purified analogous to Example 1 B) yielding a DNA-fragment with 543 bp.

C) The further handling was carried out analogous to Example 1 B), E)-L) yielding the plasmid pNCO-Lys165-BS-LuSy.

D) The SDS-PAGE was carried out analogous to Example 1 M). In the crude lysate of the strain XL1-pNCO-Lys165-BS-LuSy an overexpressed protein band with a molecular weight of circa 17 kDa could be observed which was not detectable in a strain without the plasmid XL1-pNCO-Lys165-BS-LuSy. The expression rate of this protein could be estimated to 10% (related to all soluble cell proteins).

E) The further analytical experiments were carried out analogous to Example 1 N)-Q). No significant differences to the wild type lumazine synthase could be observed with Lys165-BS-LuSy.

### Example 16

Extension of the C-Terminus of the Lumazine Sythase from Bacillus subtilis and Introduction of a Amino Acid Residue with a SH-Group (Cysteine) as a Basis for the Chemical Coupling of Target Molecules

A) The construction was carried out analogous to Example 15 A)-C), excepting that the oligonucleotide C-Cys167 (5 tat tat gga tcc tta gca gcc acc acc aga gcc acc acc aga acc acc gcc acc ttc gaa aga acg gtt taa gtt tgc cat ttc 3′), which bound at its 3′-end to the 3′-end of the ribH gene and which introduced in close contact to the last coding base triplett of the ribH gene a DNA sequence coding for the peptide (Gly)₄Ser-(Gly)₃Ser-G1_{Y3}-Cys was used as reverse primer and the plasmid pNCO-BS-LuSy (Example 1) was used as template for the PCR. Directly after the base triplett coding for the cysteine residue (tgc) at position 167 in the arteficial protein, a stop codon and a recognition site for the restriction endonuclease BamHI was introduced.

B) The PCR mixture was analyzed and purified analogous Example to 1 B) yielding a DNA-fragment with 549 bp.

C) The further handling was carried out analogous to Example 1 B), E)-L) yielding the plasmidpNCO-Cys167-BS-LuSy.

D) The SDS-PAGE was carried out analogous to Example 1 M). In the crude lysate of the strain XL1-pNCO-Cys167-BS-LuSy an overexpressed protein band with a molecular weight of circa 17.1 kDa could be observed which was not detectable in a strain without the plasmid XL1-pNCO-Cys167-BS-LuSy. The expression rate of this protein could be estimated to 5% (related to all soluble cell proteins).

E) The further characterization was carried out analogous to Example 1 N)-Q). No significant differences to the wild type lumazine synthase could be observed with Cys167-BS-LuSy.

### Example 17

Extension of the N-Terminus of the Lumazine Synthase from Bacillus subtilis via Introduction of a Peptide (12 Amino Acid Residues, FLAG-Tag) Serving as an Epitop for a Monoclonal Antibody (Anti-FLAG-M2/IgG1/Maus)

A) The gene coding for the lumazine synthase from Bacillus subtilis was amplified analogous Example 1 A), excepting that the oligonucleotide FLAG-BS-LuSy-1 (5′ ata ata ata aag ctt atg aat atc ata caa gga aat tta g 3′), which bound at its 3′-end to the 5′-end of the ribH gene and which introduced a recognition site for the restriction endonuclease HindIII (A*AGCTT) at the 5′-end, was used as forward primer and oligonucleotide Flag-BS-LuSy-2 (5′ tat tat gaa ttc tta ttc gaa aga acg gtt taa g 3′), which bound at its 3′-end to the 3′-end of the ribH gene and which introduced a recognition site for the restriction endonuclease EcoRI (G*AATTC), was used as reverse primer. The plasmid pRF2 (Example 1 A)) served as template for the PCR.

B) The PCR mixture was analyzed and purified analogous Example 1 B) yielding a DNA-fragment with 492 bp.

C) In a first step the isolated DNA fragment from B) and the vector pFLAG-MAC (Eastman Kodak Company, New Haven) were digested using the restriction endonuclease HindIII.

30.0 μl DNA pFLAG-MAC [5 μg] resp. 30 μl DNA fragment from B)

3.0 μl HindIII [60 U]

10.0 μl OPAU (10×)

57.0 μl H₂O_{bidest}

The enzymes was purchased from New England Biolabs (Schwalbach, Germany). The mixture was incubated for 150 min at 37° C. After the incubation the mixtures were purified as described under Example 1 B) and used for the digestion with the restriction endonuclease EcoRI.

D) In a second step the purified DNA fragments from C) were digested with the restriction endonuclease EcoRI.

30.0 μl DNA fragments from C)

3.0 μl EcoRI [60 U]

24.0 μl OPAU (10×)

63.0 μl H₂O_{bidest}

The enzymes were purchased from New England Biolabs (Schwalbach, Germany). The mixture was incubated for 150 min at 37° C. After the incubation the mixture was purified as described under Example 1 B) and used in a ligation protocol.

E) The further handling was carried out analogous to Example 1 G)-L) yielding the plasmid pFLAG-MAC-BS-LuSy.

F) The SDS-PAGE was carried out analogous to Example 1 M). In the crude lysate of the strain XL1-pFLAG-MAC-BS-LuSy an overexpressed protein band with a molecular weight of circa 17.7 kDa could be observed, which was not detectable in a strain without the plasmid pFLAG-MAC-BS-LuSy. The expression rate of this protein could be estimated to 10% (related to all soluble cell proteins).

G) The enzymatic activity was measured according to Example 1 N), the protein concentration was determined according to Example 1 O). Using these data a specific activity of circa 3700 U/mg could be calculated in the crude lysate which was campareable with recombinant wild type lumazine synthase.

H) Negative staining experiments were carried out analogous to Example 1 P) yielding comparable results.

I) To check the binding properties of the fused FLAG-Peptide a Western blot analysis analogous Example 1 Q) was carried out, excepting that the monoclonal antibody Anti-FLAG®M2′ (Eastman Kodak Company, New Haven) was used as primary antibody (10 μl Anti-FLAG®M2 in 5 ml TBS (50 mM Tris, 150 mM NaCl, pH 7.4)) and the monoclonal antibody Anti-mouse-IgG-HRP-conjugate (10 μl Anti-mouse-IgG-HRP-conjugate (Sigma, Munich, Germany) in 5 ml TBS; see Example 18H)) was used as secondary antibody. After visualization the fusion protein with a molecular weight of circa 17.7 kDa could be detected.

J) The purification of the fusion protein (FLAG-MAC-BS-LuSy) was carried out analogous to Example 2 S), excepting that no lysozyme was added to the lysis buffer.

K) Negative staining experiments analogous to Example 1 P) showed hollow spherical particles with an outer diameter of circa 15 nm and an inner diameter of circa 5 nm.

L) The analysis of the quarternary structure was carried out analogous to Example 1 S). The fusion protein migrated as a single band with minor changed mobility compared to wild type lumazine synthase based on the slightly increased diameter.

### Example 18

Linking of a Peptide (6 Histidin Residues; HIS6-Peptide), which can Serve as an Affinity Tag for the Binding to a Ni-Chelator Affinity Matrix or to a Monoclonal Antibody (Penta-His-Antibody) or to a Ni-NTA-HRP-Conjugate, to the C-Terminus of the Lumazine Synthase from Bacillus subtilis

A) The gene coding for the lumazine synthase from Bacillus subtilis was amplified analogous to Example 1 A), excepting that the oligonucleotide RibH-His6-C-1 (5′ gtg gtg atg gtg atg ttc gaa aga acg gtt taa g 3′), which bound at its 3′-end to the 3′-end of the ribH gene and which introduced directly after the last coding base triplett of the ribH gene a DNA fragment coding for a part of the HIS6-Peptide, was used as reverse primer. The plasmid pRF2 (see Example 1 A)) served as template for the PCR.

B) The PCR mixture was analyzed and purified analogous to Example 1 B) yielding a DNA-fragment with 492 bp and served as template for a second PCR step.

C) The second PCR step was carried out analogous to Example 1 C), excepting that the oligonucleotides EcoRI-RBS-2 (Example 2 A) was used as forward primer and oligonucleotide RibH-His6-C-2 (5′ tat tat gga tcc tta atg gtg gtg atg gtg atg 3′), which extended the DNA sequence coding for the HIS6-peptide in 3′-direction and which introduced a stop codon directly after this coding sequence and which introduced a recognition site for the restriction endonuclease BamHI (G*GATCC), was used as reverse primer.

D) The PCR mixture was analyzed and purified analogous Example 1 B) yielding a DNA-fragment with 528 bp.

E) The further handling was carried out analogous to Example 1 E)-L) yielding the Escherichia coli expression strain XL1-pNCO-C-His6-BS-LuSy.

F) The enzymatic activity was measured according to Example 1 N), the protein concentration was determined according to Example 1 O). Using these data a specific activity of circa 3700 U/mg could be calculated in the crude lysate which was campareable with recombinant wild type lumazine synthase.

G) To check the binding properties of the fused HIS6-Peptide a Western blot analysis analogous Example 1 Q) was carried out, excepting that the monoclonal antibody ‘Penta-His™ Antibody’ (Qiagen, Hilden, Germany) was used as primary antibody (10 μl ‘Penta-His™ Antibody’ in 5 ml TBS (Example 14 I)) and the monoclonal antibody Anti-mouse-IgG-HRP-conjugate (10 μl Anti-mouse-IgG-HRP-conjugate in 5 ml TBS; Example 17 I)) was used as secondary antibody. After visualization the fusion protein could be detected at circa 17.1 kDa.

H) To check the accessibility of the HIS6-Peptide on the surface of the icosahedron an ELISA protocol (enzyme linked immunosorbent assay) was carried out on 96 well microtiter plates analogous to Example 14 M)), excepting that the first well of the microtiter plate was filled with 100 μl crude lysate from E) (5-8 mg/ml protein in the crude lysate). 50 μl Dilution buffer (1% skimmed milk powder in PBS) was added to the wells 2-8 in the same column. In a subsequent step 50 μl of the protein solution in the first well was removed and added to the solution in well 2 and mixed with the dilution buffer. 50 μl of this diluted protein solution from well 2 was removed and added to the solution in well 3 and mixed. 50 μl from 3 to 4, 50 μl from 4 to 5, 50 μl from 5 to 6, 50 μl from 6 to 7, 50 μl from 7 to 8 and 50 μμl from 8 to waste (dilution: log 2). The samples were incubated overnight at 37° C. Afterwards the solution in the wells was removed totally and the wells were washed 3× with 350 μl PBS. 350 μl Solution A (3% skimmed milk powder in PBS buffer) were added to each well and incubated for 1 h at 37° C. Subsequently Solution A was removed totally and each well was washed 3× with 350 μl PBS. Afterwards 10 μl Penta-His™ Antibody (Qiagen, Hilden) were mixed with 5 ml Dilution buffer (1. Antibody solution). To each well 50 μl of the 1. Antibody solution were added and the mixture was incubated 2 h at 37° C. Afterwards the solution was removed totally and the wells were washed 3× with 350 μl PBS. In a further step 150 μl of the 2. Antibody solution (10 μl Anti-mouse-IgG-HRP-conjugate in 5 ml Dilution buffer) were filled in each well and the mixture was incubated 2 h at 37° C. Afterwards the solution was removed totally and the wells were washed 3× with 350 μl PBS. For the visualization 150 μl Substrate solution (100 mg o-Phenylendiamine (Sigma, Munich, Germany) in 25 ml Substrat buffer; Substrate buffer: 50 mM citric acid, pH 5) were added to each well and incubated at room temperature. The extinction was measured at 492 nm in an ELISA reader. The results showed that the HIS6-Peptides are located on the surface of the lumazine synthase fusion protein. Based on the log 2 dilution of the target protein (C-His6-BS-LuSy), a decrease in the signal intensity could be observed.

I) Negative staining experiments analogous to Example 1 P) showed hollow spherical particles with an outer diameter of circa 15 nm and an inner diameter of circa 5 nm.

J) The analysis of the quarternary structure was carried out analogous to Example 1 S). The fusion protein migrated as a single band with changed mobility compared to wild type lumazine synthase based on the slight increase of the diameter.

### Example 19

Preparation of a Mixed Lumazine Synthase Conjugate (Hetero-Oligomeric Lumazine Synthase Conjugates) Consisting of the Lumazine Synthase Fusion Proteins C-Biotag-BS-LuSy and C-His6-BS-LuSy Using an in Vitro Refolding Protocol

A) An Escherichia coli XL1 host strain carrying the expression plasmid pNCO-C-Biotag-BS-LuSy (Example 14) was fermented analogous to Example 1 K, excepting that 500 ml medium was used.

B) An Escherichia coli XL1 host strain carrying the expression plasmid pNCO-C-His6-BS-LuSy (Example 18) was fermented analogous to Example 1 K, excepting that 500 ml medium was used.

C) Cells from A) were thawed and lysed using a ultrasonic device from Branson SONIC Power Company (Branson-Sonifier B-12A, Branson SONIC Power Company, Dunbury, Conn.). The cell pellet from A) was suspended in 40 ml Separation buffer (50 mM Tris pH 9.5) and cooled on ice for 10 min. The cell suspension was then lysed using the ultrasonic device (15 pulses at level 5). The suspension was then cooled on ice for 5 min and lysed under the same conditions again. The treatment was repeated 4 times. After the last sonication the suspension was centrifuged (Sorvall-centrifuge with SS34-rotor; 5000 rpm, 4° C., 10 min), the supernatant (crude lysate A-1) was removed and the cell pellet (cell pellet A-1) was used for the following steps.

D) The lysis was carried out for a second time analogous to C), whereas the cell pellet A-1 was suspended in 40 ml Separation buffer, yielding the crude lysate A-2 and the cell pellet A-2.

E) Cells from B) were thawed and lysed using a ultrasonic device from Branson SONIC Power Company (Branson-Sonifier B-12A, Branson SONIC Power Company, Dunbury, Conn.). The cell pellet from B) was suspended in 40 ml Separation buffer (50 mM Tris pH 9.5) and incubated on ice for 10 min. The cell suspension was then lysed using the ultrasonic device (15 pulses at level 5). The suspension was then cooled on ice for 5 min and lysed under the same conditions again. The treatment was repeated 4 times. After the last sonication the suspension was centrifuged (Sorvall-centrifuge with SS34-rotor; 15000 rpm, 4° C., 10 min) and the supernatant (crude lysate B) was used for the following steps.

F) The cell pellet A-2 (C-Biotag-BS-LuSy) from D) was solubilized in 40 ml Solubilization buffer (50 mM Tris, pH 9.5, 6 M guanidinium thiocyanate (G-SCN), 100 mM dithiothreitole (DTT)) for 24 h at room temperature yielding the Solubilization solution.

G) To crude lysate B (40 ml) 6 M G-SCN and 100 mM DTE were added and the mixture was incubated for 24 h at room temperature.

H) The Solubilization solution from F) was centrifuged (Sorvall-centrifuge with SS34-rotor; 15000 rpm, 25° C., 20 min) yielding the supernatant A-3 and the cell pellet A-3.

I) Afterwards aliquots of supernatant A-3 and cell pellet A-3 were analyzed using a SDS-PAGE.

J) The supernatant was checked analogous Example 1 M).

K) For the analysis of cell pellet A-3 a small aliquot of cell pellet A-3 was suspended in 200 μl Sample buffer (Example 1 M) and boiled for 15 min. Afterwards the suspension was centrifuged (Eppendorff centrifuge, 15000 rpm, 5 min, 4° C.) and 6 μl of the clear supernatant was applied to a SDS-PAGE. The further handling was carried out analogous to Example 1 M).

L) The data from J) and K) showed that the insoluble protein C-Biotag-BS-LuSy could be solubilized to 80% under the described conditions.

M) The cell pellet A-3 from H) was treated again following the steps F) and H)-K). The amount of soluble material couldn't be increased.

N) The concentrations of the fusion proteins from E) (crude lysate B) and H) (supernatant A-3) were in the same range (related to the amount of target protein).

O) 2 ml of supernatant-C-Bio-BS-LuSy (supernatant A-3) and 2 ml of supernatant-C-His6-BS-LuSy (crude lysate B) were mixed (Mixture A).

P) 3.5 ml of supernatant-C-Bio-BS-LuSy (supernatant A-3) and 0.5 ml of supernatant-C-His6-BS-LuSy (crude lysate B) were mixed (Mixture B).

Q) Mixture A and Mixture B were stirred for 48 h at room temperature.

R) Mixture A and Mixture B from Q) were dialysed against 400 ml Separation buffer containing 8 M urea and 1 mM DTE for 18 h at room temperature.

S) 6.6 mM 5-Nitro-6-(D-ribitylamino)2,4-(1H,3H)-pyrimidindione were added to Mixture A and Mixture B from S) yielding Mixture-A-Nitro and Mixture-B-Nitro. The solutions were stirred for 8 h at room temperature.

T) Mixture-A-Nitro and Mixture-B-Nitro were dialysed against 32 ml (5×volume) Refolding buffer A (100 mM K-phosphate buffer, pH 7.0, 1 mM 5-Nitro-6-(D-ribitylamino)2,4-(1H,3H)-pyrimidindione, 1 mM DTE, 0.02% Na-azide) for 12 h at 4° C. yielding Mixture-A-1/5 and Mixture-B-1/5.

U) In a subsequent step the Refolding buffer A from T) was diluted with 40 ml Refolding buffer B (100 mM K-phosphate buffer, pH 7.0, 1 mM DTE, 0.02% Na-azide) and the dialysis was carried out for further 24 h at 4° C. yielding Mixture-A-1/10 and Mixture-B-1/10.

V) In a subsequent step the Refolding buffer B from U) was diluted with 80 ml Refolding buffer B and the dialysis was carried out for further 24 h at 4° C. yielding Mixture-A-1/20 and Mixture-B-1/20.

W) Afterwards Mixture-A-1/20 and Mixture-B-1/20 were dialysed for 24 h at 4° C. against 72 ml (10 fold volume) Refolding buffer C (100 mM K-phosphate buffer, pH 7.0, 0.25 mM 5-Nitro-6-(D-ribitylamino)2,4-(1H,3H)-pyrimidindione, 1 mM DTE, 0.02% Na-azide) yielding Mixture-A-1/200 and Mixture-B-1/200.

X) Mixture-A-1/200 and Mixture-B-1/200 were centrifuged (Sorvall-centrifuge with SS34-rotor; 15000 rpm, 4° C., 20 min).

Y) Aliquots of the supernatants were analyzed using a SDS-PAGE. The analysis of the supernatants was carried out analogous to Example 1 M).

Z) The analysis of the pellets resulting from X) was carried out analogous to K).

AA) The analysis from Y) and Z) showed that mixed lumazine synthase conjugates could be generated in an amount of 50-80% by the described protocol. The amounts of the used protein concentrations at the beginning of the refolding process corresponded to the amounts of each protein in the analyzed conjugates. No difference in the refolding behaviour of both proteins could be observed.

BB) To check the quarternary structure of the lumazine synthase conjugates an experiment analogous to Example 1 S) was carried out, excepting that 40 μl protein solution (Supernatant Mixture-A-1/200; Supernatant Mixture-B-1/200) were used for the electrophoresis. The refolded protein conjugates could be observed as single bands on the native polyacrylamide gel. The mobility of both proteins was comparable with the mobilities of the proteins C-Bio-BS-LuSy and C-His6-BS-LuSy based on the similar hydrodynamic diameter of the four proteins. Using the described protocol above refolded mixed lumazine synthase conjugates containing different fusion partners could be obtained.

CC) To check the presence of both fusion partners on the surface of a discret lumazine synthase conjugate an ELISA protocol (enzyme linked immunosorbent assay) was carried out on 96 wells microtiter plates. Coating of the micro titer wells with avidin was carried out analogous to Example 14 M). After the coating process 100 μl (circa 0.5 to 1 mg/ml) of the refolded protein from X) were filled in the 1. well of a column. 50 μl Dilution buffer was added to the wells 2-8 in the same column. In a subsequent step 50 μl of the protein solution in the first well was removed and added to the solution in well 2 and mixed with the dilution buffer. 50 μl of this diluted protein solution from well 2 was removed and added to the solution in well 3 and mixed. 50 μl from 3 to 4, 50 μl from 4 to 5, 50 μl from 5 to 6, 50 μl from 6 to 7, 50 μl from 7 to 8 and 50 μl from 8 to waste (dilution: log 2). The samples were incubated overnight at 37° C. Afterwards the solution in the wells were removed totally and the wells were washed 3× with 350 μl PBS. 350 μl Solution A (3% skimmed milk powder in PBS buffer) were added to each well and incubated for 1 h at 37° C. Subsequently the Solution A was removed totally and each well was washed 3× with 350 μl PBS. Afterwards 10 μl Penta-His™ Antibody (Quiagen, Hilden) were mixed with 5 ml Dilution buffer (1. Antibody solution). To each well 50 μl of the 1. Antibody solution were added and the mixture was incubated 2 h at 37° C. Afterwards the solution was removed totally and the wells were washed 3× with 350 μl PBS. In a further step 150 μl of the 2. Antibody solution (10 μl Anti-mouse-IgG-HRP-conjugate in 5 ml Dilution buffer) were filled in each well and the mixture was incubated 2 h at 37° C. Afterwards the solution was removed totally and the wells were washed 3× with 350 μl PBS. For the visualization 150 μl Substrate solution (100 mg o-Phenylendiamin in 25 ml Substrat buffer; Substrate buffer: 50 mM citric acid, pH 5) were added to each well and incubated at room temperature. The extention was measured at 492 nm in an ELISA reader. The data showed that the hetero-oligomeric lumazine synthase conjugates could be bound to the avidin via biotin molecules on the surface of the icosahedron. On the other hand HIS6-Peptides could be detected via the highly specific Penta-His-Antibody on the protein conjugates which were bound to the avidin coated microtiter plate via biotin. Based on the log 2 dilution of the target protein (C-His6-BS-LuSy), a decrease in the signal intensity could be observed. Supernatant mixture A-1/200 (estimated: 30 HIS6-Peptides) showed a more intensive signal than Supernatant mixture B-1/200 (estimated: 15 HIS6-Peptides). For the binding of the hetero-oligomeric lumazine synthase conjugate to the avidin coated microtiter plate, just one single biotin molecule was needed. In the Supernatant mixture A-1/200 more HIS6-Peptides (30) were presented on the surface of the icosahedron than in the Supernatant mixture B-1/200 (15). Based on this fact the signal resulting from the binding of the Penta-His-Antibody should be more intensive in the Supernatant mixture A-1/200.

Using the described protocol above refolded mixed lumazine synthase conjugates (heterooligomeric lumazine synthase conjugates) containing different fusion partners, whereby the fusion peptides are located on the surface of the icohedron, could be obtained.

### Example 20

Construction of a Synthetic Gene Coding for a Thermostable Lumazine Synthase Based on the Hyperthermophilic Bacterium Aquifex aeolicus (Deckert et al., 1998) 11 Oligonucleotides Adapted to the Escherichia coli Codon Usage for Highly Expressed Proteins Served as Primers in a 6-Phase PCR

A) The gene coding for the lumazine synthase from Aquifex aeolicus was amplified using the oligonucleotide AQUI-1 (5′ gct gcg ggt gaa ctg gcg cgt aaa gag gac att gat gct gtt atc gca att ggc gtt ctc atc 3′) as forward primer and oligonucleotide AQUI-2 (5′ cta atg aaa ggt tcg cga ggc ctt ttg aaa ctt cag agg cga tat aat cga aat gtg gcg ttg 3′) as reverse primer. Each of the oligonucleotides has been adapted to the Escherichia coli codon usage for highly expressed proteins (Grosjean und Fiers, 1982; Ikemura, 1981; Wada et al. 1992). The oligonucleotide AQUI-1 contained a recognition site for the restriction endonuclease MfeI (C*AATTG) and the oligonucleotide AQUI-2 contained a recognition site for the restriction endonuclease StuI (AGG*CCT). The plasmid pNCO-BS-LuSy (Example 1) served as template for the PCR.

10 μl PCR-buffer (75 mM Tris/HCl, pH 9.0; 20 mM (NH₄)₂SO₄; 0.01% (w/v) Tween 20)

6 μl Mg²⁺[1.5 mM]

8 μl dNTP's [je 200 μM]

1 μl AQUI-1 [0.5 μM]

1 μl AQUI-2 [0.5 μM]

1 μl pNCO-BS-LuSy [10 ng]

1 μl Goldstar-Taq-Polymerase [0.5 U] (Eurogentec, Seraing, Belgien)

72 μl H₂O_{bidest}

PCR cycle protocol (GeneAmp® PCR System 2400; Perkin Elmer):

1. 5.0 min 95° C.

2. 0.5 min 94° C.

3. 0.5 min 50° C.

4. 0.5 min 72° C.

5. 7.0 min 72° C.

6. ∞ 4° C.

Steps 2.-4. were repeated 20 times.

B) The PCR mixture was analysed and purified analogous Example 1 B), excepting that an agarose gel was used containing 3% agarose, yielding a DNA-fragment with a length of 132 bp.

C) 10 ng of the purified DNA from B) served as a template for a 2. PCR using the oligonucleotide AQUI-3 (5′ act ctg gtt cgt gtt cca ggc tca tgg gaa ata ccg gtt gct gcg ggt gaa ctg gcg cgt aaa g 3′), which was identical to the 5′-end of primer AQUI-1 and the oligonucleotide AQUI-4 (5′ cca agg tgt cag ctg taa taa cac cga agg tga tag gtt tac gta gtt cta atg aaa ggt tcg cga ggc c 3′), which was identical to the 5′-end of primer AQUI-2, as forward and as reverse primers. The oligonucleotide AQUI-3 contained a recognition site for the restriction endonuclease AgeI (A*CCGGT) and the oligonucleotide AQUI-4 a recognition site for the restriction endonucleases SnaBI (TAC*GTA) and PvuII (CAG*CTG). The PCR was carried out analogous to A).

D) The PCR mixture was analysed and purified analogous B) yielding a DNA-fragment with a length of 219 bp.

E) 10 ng of the purified DNA from D) served as a template for a 3. PCR using the oligonucleotide AQUI-5 (5′ gga ggg tgc aat tga ttg cat agt ccg tca tgg cgg ccg tga aga aga cat tac tct ggt tcg tgt tcc agg c 3′), which was identical to the 5-end of primer AQUI-3 and the oligonucleotide AQUI-6 (5′ gtt gcc gtg ttt tgt gcc ggc gcg ctc gat agc ctg ttc caa ggt gtc agc tgt aat aac 3′), which was identical to the 5′-end of primer AQUI-4, as forward and as reverse primers. The oligonucleotide AQUI-5 contained a recognition site for the restriction endonuclease EagI (C*GGCCG) and the oligonucleotide AQUI-6 a recognition site for the restriction endonucleases BssHII (G*CGCGC) and PvuII (CAG*CTG). The PCR was carried out analogous to A).

F) The PCR mixture was analysed and purified analogous B) yielding a DNA-fragment with a length of 309 bp.

G) 10 ng of the purified DNA from F) served as a template for a 4. PCR using the oligonucleotide AQUI-7 (5′ cgg tat cgt agc atc acg ttt taa tca tgc tct tgt cga ccg tct ggt gga ggg tgc aat tga ttg cat ag 3′), which was identical to the 5′-end of primer AQUI-5 and the oligonucleotide AQUI-8 (5′ gaa taa gtt tgc cat ttc aat ggc aga aag cgc tgc ttc cca acc ttt gtt gcc gtg ttt tgt gcc ggc 3′), which was identical to the 5′-end of primer AQUI-6, as forward and as reverse primers. The oligonucleotide AQUI-7 contained a recognition site for the restriction endonuclease SalI (G*TCGAC) and the oligonucleotide AQUI-8 a recognition site for the restriction endonuclease Eco56I (G*CCGGC). The PCR was carried out analogous to A).

H) The PCR mixture was analysed and purified analogous B) yielding a DNA-fragment with a length of 405 bp.

I) 10 ng of the purified DNA from H) served as a template for a 5. PCR using the oligonucleotide AQUI-9 (5′ atg caa atc tac gaa ggt aaa cta act gct gaa ggc ctt cgt ttc ggt atc gta gca tca cgt ttt aat c 3′), which was identical to the 5′-end of primer AQUI-7 and the oligonucleotide AQUI-10 (5′ tat tat gga tcc tta tcg gag aga ctt gaa taa gtt tgc cat ttc aat gg 3′), which was identical to the 5′-end of primer AQUI-8, as forward and as reverse primers. The oligonucleotide AQUI-9 contained a recognition site for the restriction endonuclease StuI (AGG*CCT) and the oligonucleotide AQUI-10 introduced a recognition site for the restriction endonuclease BamHI (G*GATCC) directly after the stop codon of the gene coding for the lumazine synthase from Aquifex aeolicus. The PCR was carried out analogous to A).

J) The PCR mixture was analysed and purified analogous B) yielding a DNA-fragment with a length of 476 bp.

K) 10 ng of the purified DNA from J) served as a template for a 6. PCR using the oligonucleotide AQUI-11 (5′ ata ata gaa ttc att aaa gag gag aaa tta act atg caa atc tac gaa ggt aaa cta ac 3′), which was identical to the 5′-end of primer AQUI-9 and which coded for an optimized ribosome binding site at its 5′-end and the oligonucleotide AQUI-10, as forward and as reverse primers. The oligonucleotide AQUI-11 contained a recognition site for the restriction endonuclease EcoRI (G*AATTC) upstream to the ribosome binding site. The PCR was carried out analogous to A).

L) The PCR mixture was analysed and purified analogous B) yielding a DNA-fragment with a length of 510 bp.

M) The further handling was carried out analogous to Example 1 E) to G) yielding the plasmid pNCO-AA-LuSy.

N) The further handling of the plasmid pNCO-AA-LuSy was carried out analogous to Example 1H) to M). In the crude lysate of the strain XL1-pNCO-AA-LuSy a protein band with a molecular weight of circa 16.7 kDa could be observed. This protein band couldn't be observed in a Escherichia coli strain without the expression plasmid pNCO-AA-LuSy. The observed protein band corresponded to circa 20% of the total soluble proteins of the Escherichia coli strain.

O) The enzymatic activity was measured according to Example 1 N), whereby the protein showed an activity optimum in a temperature range of 80-90° C.

P) Negative staining experiments were carried out analogous to Example 1 P) yielding spherical hollow protein particles with an outer diameter of circa 15 nm and an inner diameter of circa 5 nm.

Q) The isolation of the lumazine synthase coded by the described synthetic gene was carried out in two steps: The fermentation of the Escherichia coli strain XL1-pNCO-AA-LuSy was carried out analogous to Example 1 K), excepting that 1 l medium was used. The lysis of the resulting cells was carried out analogous to Example 1 R). The supernatant after the centrifugation (crude lysate) was treated at 90° C. for 20 min in a water bath. Subsequently the resulting suspension was centrifuged (Sorvall SS34-rotor; 15000 rpm; 4° C.; 30 min) and the supernatant was used for the further experiments. The resulting recombinant protein was 80% pure after this step. The further purification was carried out analogous to Example 2 S) using a gelfiltration column.

R) The quarternary check was carried out analogous to Example 1 S) yielding a result comparable to the lumazine synthase capsids from Bacillus subtilis.

### Example 21

Linking of a Peptide (13 Aminoacid Residues; Bio-Peptide), which can be Biotinylated in Vivo to the C-Terminus of the Lumazine Synthase (ribH) from Aquifex aeolicus (see Example 20 and 14)

A) The gene coding for the lumazine synthase from Aquifex aeolicus was amplified analogous to Example 1 A), excepting that the oligonucleotide EcoRI-RBS-2 (Example 2 A)) was used as forward primer and oligonucleotide AQUI-C-NotI (5′ tat tat tat agc ggc cgc tcg gag aga ctt gaa taa g 3′) was used as reverse primer. The oligonucleotide AQUI-C-NotI was at its 3′-end identical to the 3′-end of the ribH gene and introduced a recognition site for the restriction endonuclease NotI (GC*GGCCGC) directly after the last coding base triplett. The DNA sequence representing the recognition site for the endonuclease was translated into three alanine residues. The plasmid pNCO-AA-LuSy (Example 20) served as template for the PCR.

B) The PCR mixture was analyzed and purified analogous to Example 1 B) yielding a DNA-fragment with 513 bp.

C) The DNA fragment from B) was digested with the restriction endonuclease NotI analogous to Example 8 F). After incubation the DNA fragment was purified analogous Example 1 B).

D) The DNA fragment from C) was digested with the restriction endonuclease EcoRI analogous to Example 5H). After incubation the DNA fragment was purified analogous Example 1 B) yielding a DNA fragment with a length of 498 bp.

E) 5 μg of the expression plasmid pNCO-C-Biotag-BS-LuSy (Example 14), in a volume of 30 μl were treated analogous to Example 8 G) and H). The DNA fragment with a length of 3437 bp was isolated analogous to Example 1 B).

F) The further handling was carried out analogous to Example 1 G) to L) yielding the Escherichia coli expression strain XL1-pNCO-C-Biotag-AA-LuSy.

G) The SDS-PAGE was carried out analogous to Example 1 M). In the crude lysate of the strain XL1-pNCO-C-Biotag-AA-LuSy no overexpressed protein band with a molecular weigth of circa 18.5 kDa could be observed.

H) The further handling was carried out analogous to Example 14H) to M) yielding comparable results.

### Example 22

Linking of a Peptide (13 Aminoacid Residues; Bio-Peptide), which can be Biotinylated in Vivo, via a Linker Peptide Consisting of the Aminoacid Residues H-H-H-H-H-H-A-A-A to the C-Terminus of the Thermostable Lumazine Synthase (ribH) from Aquifex aeolicus

A) The gene coding for the lumazine synthase from Aquifex aeolicus was amplified analogous to Example 1 A), excepting that the oligonucleotide EcoRI-RBS-2 (Example 2 A)) was used as forward primer and oligonucleotide AQUI-C-HIS₆-NotI (5′ tat tat tat agc ggc cgc atg gtg gtg atg gtg atg tcg gag aga ctt gaa taa gtt tgc 3′) was used as reverse primer. The oligonucleotide AQUI-C-HIS₆-NotI was at its 3′-end identical to the 3′-end of the ribH gene and introduced directly after the last coding base triplett of the ribH gene a sequence coding for 6 histidine residues and directly after this sequence a recognition site for the restriction endonuclease NotI (GC*GGCCGC). The DNA sequence representing the recognition site for the endonuclease was translated into three alanine residues. The plasmid pNCO-AA-LuSy (Example 20) served as template for the PCR.

B) The PCR mixture was analyzed and purified analogous to Example 1 B) yielding a DNA-fragment with 531 bp.

C) The DNA fragment from B) was digested with the restriction endonuclease NotI analogous to Example 8 F). After incubation the DNA fragment was purified analogous Example 1 B).

D) The DNA fragment from C) was digested with the restriction endonuclease EcoRI analogous to Example 8 H). After incubation the DNA fragment was purified analogous Example 1 B) yielding a DNA fragment with a length of 516 bp.

E) The expression vector was treated analogous to Example 21 E).

F) The further handling was carried out analogous to Example 1 G) to J) yielding the Escherichia coli expression strain XL1-pNCO-HIS6-C-Biotag-AA-LuSy.

G) The fermentation of the cells was carried out analogous to Example 1 R). After the centrifugation the clear supernantant was removed and the resulting cell pellet was used for the further experiments.

H) The insoluble pellet from G) was incubated in 50 ml NTA-buffer-A (50 mM Na-phosphate-buffer pH 8.0, 300 mM NaCl, 0.02% Na-azide, 6 M guanidiniumhydrochloride) for 24 h at room temperature whereby the solution was stirred. Afterwards the suspension was centrifuged (Sorvall SS34-Rotor, 15000 rpm, 20° C., 20 min). The resulting supernatant was removed and used for the further experiments. The supernatant was mixed with 6 ml Ni-NTA-agarose (Qiagen, Hilden, Germany) and incubated overnight in a waver (20° C.). Afterwards the suspension was centrifuged (800 g, 20° C., 10 min). The supernatant was removed and the resulting pellet was suspended in 10 ml NTA-buffer-A and incubated for 15 min in a waver (20° C.). The suspension was centrifuged again and the supernatant was removed. The pellet was suspended in 10 ml NTA-buffer-B (8 M urea, 100 mM Na-phosphat-buffer, 10 mM Tris pH 6.3) and incubated for 15 min in a waver (20° C.) and subsequently centrifuged. The treatment was repeated once more. Subsequently the resulting pellet was washed twice using each 10 ml NTA-buffer-C (8 M urea, 100 mM Na-phosphat-Puffer, 10 mM Tris pH 5.9). At least the pellet was treated twice using each 10 ml NTA-Puffer-D (8 M urea, 100 mM Na-phosphat-buffer, 10 mM Tris pH 4.5). The pollutions could be removed using NTA-buffer-B and the target protein could be eluted by the use of NTA-buffer-C and NTA-buffer-D. After neutralization of the fractions a SDS-PAGE was carried out. On the polyacrylamide gel just one single band with a molecular weight of 19.3 kDa could be observed.

### Example 23

Linking of a Peptide (13 Aminoacid Residues; Bio-Peptide), which can be Biotinylated in Vivo, via a Linker Peptide Consisting of the Aminoacid Residues H-H-H-H-H-H-G-G-S-G-A-A-A to the C-Terminus of the Thermostable Lumazine Synthase (ribH) from Aquifex aeolicus

A) The gene coding for the lumazine synthase from Aquifex aeolicus was amplified analogous to Example 21), excepting that the oligonucleotide EcoRI-RBS-2 (Example 2 A)) was used as forward primer and oligonucleotide AQUI-C-HIS₆-GLY₂)-SER-GLY-NotI (5′ tat tat tat agc ggc cgc gcc aga acc gcc atg gtg gtg atg gtg atg tcg gag aga ctt gaa taa gtt tgc 3′) was used as reverse primer. The oligonucleotide AQUI-C-HIS₆-GLY₂-SER-GLY-NotI was at its 3′-end identical to the 3′-end of the ribH gene and introduced directly after the last coding base triplett of the ribH gene a sequence coding for the peptide H-H-H-H-H-H-G-G-S-G and directly after this sequence a recognition site for the restriction endonuclease NotI (GC*GGCCGC). The DNA sequence representing the recognition site for the endonuclease was translated into three alanine residues. The plasmid pNCO-AA-LuSy (Example 20) served as template for the PCR. The PCR was carried out analogous to Example 1 A).

B) The PCR mixture was analyzed and purified analogous to Example 1 B) yielding a DNA-fragment with 543 bp.

C) The DNA fragment from B) was digested with the restriction endonuclease NotI analogous to Example 8 F). After incubation the DNA fragment was purified analogous Example 1 B).

D) The DNA fragment from C) was digested with the restriction endonuclease EcoRI analogous to Example 8H). After incubation the DNA fragment was purified analogous Example 1 B) yielding a DNA fragment with a length of 528 bp.

E) The expression vector was treated analogous to Example 21 E).

F) The further handling was carried out analogous to Example 1 G) to J) yielding the Escherichia coli expression strain XL1-pNCO-HIS6-GL Y2-SER-GLY-C-Biotag-AA-LuSy.

G) The further handling was carried out analogous to Example 22 G) to H) yielding comparable results.

### Example 24

Construction of a Chimeric Protein Consisting of a Part of the Lumazine Synthase from Bacillus subtilis and a Part of the Thermostable Lumazine Synthase from Aquifex aeolicus

A) A part of the gene coding for the lumazine synthase from Bacillus subtilis was amplified analogous to Example 1 A), excepting that the oligonucleotide EcoRI-RBS-2 (Example 2 A)) was used as forward primer and oligonucleotide BS-LuSy-AgeI (5′ tat tat tat aac cgg tat ttc aaa tgc gcc 3′) was used as reverse primer and the plasmid pNCO-BS-LuSy (see Example 1) was used as template for the PCR. The oligonucleotide BS-LuSy-AgeI was at its 3′-end identical to a region of the ribH gene from Bacillus subtilis and introduced a recognition site for the restriction endonuclease AgeI (A*CCGGT).

B) The PCR mixture was analyzed and purified analogous to Example 1 B) yielding a DNA-fragment with 225 bp. The purified fragment from B) was digested using the restriction endonuclease AgeI.

30.0 μl DNA-fragmente from B)

4.0 μl AgeI [8 U]

10.0 μl buffer 1(10×) [10 mM Bis-Tris-Propane-HCl, 10 mM MgCl₂, 1 mM DTT, pH 7.0]

56.0 μl H₂O_{bidest}

The enzymes was purchased from New England Biolabs (Schwalbach, Germany). The mixture was incubated for 180 min at 25° C. After incubation the mixture was purified as described under Example 1 B) and used for the digestion with the restriction endonuclease EcoRI.

C) In a second step the purified DNA fragment from B) was digested with the restriction endonuclease EcoRI.

30.0 μl DNA-fragment from B)

3.0 μl EcoRI [60 U]

20.0 μl OPAU (10×)

47.0 μl H₂O_{bidest}

The enzymes was purchased from New England Biolabs (Schwalbach, Germany). The mixture was incubated for 180 min at 37° C. After incubation the mixture was purified as described under Example 1 B).

D) The plasmid pNCO-AA-LuSy (Example 20, 30 μl, 5 μg) was treated analogous to B) and C) and subsequently purified analogous to Example 1 B), yielding a DNA-fragment with 3676 bp, which was used in a ligation protocol.

E) The further handling was carried out analogous to Example 1 G) to L) yielding the Escherichia coli expression strain XL1-pNCO-BS-LuSy-AgeI-AA-LuSy.

F) Enzymatic activity could be measured according to Example 1 N).

G) To check the expression rate resp. the molecular weight of the soluble protein a SDS-PAGE was carried out analogous to Example 1 M). In the crude lysate of the strain XL1-pNCO-BS-LuSy-AgeI-AA-LuSy an overexpressed protein band with a molecular weight of circa 16.4 kDa could be observed which was not detectable in a strain without the plasmid pNCO-BS-LuSy-AgeI-AA-LuSy. The expression rate of this protein could be estimated to 10% (related to all soluble cell proteins).

### Example 25

Construction of a Vector for the Recombinant N-Terminal Fusion of Target Peptides to the Lumazine Synthase from Aquifex aeolicus (Target Peptides can be Fused Directly without the Use of a Linker Peptide to the Carrier Protein, whereby the Singular Restriction Site BglII is used, which is Located Inside the Gene Coding for the Carrier Protein)

A) The gene coding for the lumazine synthase from Aquifex aeolicus was amplified analogous Example 1 A), excepting that the oligonucleotide AQUI-11-BglII (5′ ata ata gaa ttc att aaa gag gag aaa tta act atg cag atc tac gaa gg 3′), which bound at its 3′-end to the 5′-end of the ribH gene of Aquifex aeolicus and which introduced a recognition site for the restriction endonuclease BglII (A*GATCT) via a silent mutation and which introduced a recognition site for the restriction endonuclease EcoRI (G*AATTC) at the 5′-end, was used as forward primer and oligonucleotide AQUI-10 (see Example 20) was used as reverse primer. The plasmid pNCO-AA-LuSy (Example 20) served as template for the PCR.

B) The PCR mixture was analyzed and purified analogous Example 1 B) yielding a DNA-fragment with 510 bp.

C) The further handling was carried out analogous to Example 1 E)-L) yielding the plasmid pNCO-AA-BglII-LuSy.

D) To check the expression rate resp. the molecular weight of the soluble protein a SDS-PAGE was carried out analogous to Example 1 M). In the crude lysate of the strain XL1-pNCO-AA-BglII-LuSy an overexpressed protein band with a molecular weight of circa 16.7 kDa could be observed which was not detectable in a strain without the plasmid pNCO-AA-BglII-LuSy. The expression rate of this protein could be estimated to 20% (related to all soluble cell proteins).

E) The further analytics were carried out analogous to Example 20 O)-R), whereby no significant difference related to the wild-type protein (AA-LuSy) could be observed.

### Example 26

Construction of a Vector for the C-Terminal Fusion of Target Peptides to the Lumazine Synthase from Aquifex aeolicus

A) The gene coding for the lumazine synthase from Aquifex aeolicus was amplified analogous Example 1 A), excepting that the oligonucleotide EcoRI-RBS-2 (see Example 2 A)) was used as forward primer and oligonucleotide AQUI-10-(BamHI) (5′ tat tat gga tcc tcg gag aga ctt gaa taa gtt tgc 3′), which bound at its 3′-end to the 3′-end of the ribH gene from Aquifex aeolicus and which introduced directly after the last coding base triplett a recognition site for the restriction endonuclease BamHI (G*GATCC), whereby the original stop codon was removed, was used as reverse primer. The plasmid pNCO-AA-LuSy (Example 20) served as template for the PCR.

B) The PCR mixture was analyzed and purified analogous Example 1 B) yielding a DNA-fragment with 507 bp.

C) The further handling was carried out analogous to Example 1 E)-L) yielding the plasmid pNCO-AA-LuSy-(BamHI).

D) To check the expression rate resp. the molecular weight of the soluble protein a SDS-PAGE was carried out analogous to Example 1 M). In the crude lysate of the strain XL1-pNCO-AA-LuSy-(BamHI) an overexpressed protein band with a molecular weight of circa 17.8 kDa could be observed which was not detectable in a strain without the plasmid pNCO-AA-LuSy-(BamHI). The expression rate of this protein could be estimated to 20% (related to all soluble cell proteins).

E) The further analytics were carried out analogous to Example 20 O)-R), whereby no significant difference related to the wild-type protein (AA-LuSy) could be observed.

### Example 27

Construction of a Vector for the Simultaneous N-Terminal and C-Terminal Fusion of Target Peptides to the Lumazine Synthase of Aquifex aeolicus

A) The gene coding for the lumazine synthase from Aquifex aeolicus was amplified analogous Example 1 A), excepting that the oligonucleotide EcoRI-RBS-2 (see Example 2 A)) was used as forward primer and oligonucleotide AQUI-10-(BamHI) (5′ tat tat gga tcc tcg gag aga ctt gaa taa gtt tgc 3′; Example 26) was used as reverse primer and excepting that the plasmid pNCO-AA-BglII-LuSy (Example 25) served as template for the PCR.

B) The PCR mixture was analyzed and purified analogous Example 1 B) yielding a DNA-fragment with 507 bp.

C) The further handling was carried out analogous to Example 1 E)-L) yielding the plasmid pNCO-BglII-AA-LuSy-(BamHI).

D) To check the expression rate resp. the molecular weight of the soluble protein a SDS-PAGE was carried out analogous to Example 1 M). In the crude lysate of the strain XL1-pNCO-BglII-AA-LuSy-(BamHI) an overexpressed protein band with a molecular weight of circa 17.8 kDa could be observed which was not detectable in a strain without the plasmid pNCO-AA-BglII-LuSy-(BamHI). The expression rate of this protein could be estimated to 20% (related to all soluble cell proteins).

E) The further analytics were carried out analogous to Example 20 O)-R), whereby no significant difference related to the wild-type protein (AA-LuSy) could be observed.

Literature

Altschul, S. F., T. L. Madden, A. A. Schäffer, J. Zhang, Z. Zhang, W. Miller, D. J. Lipman (1997) Gapped BLAST and Psi-BLAST: a new generation of protein database search programs Nucleic Acids Res. 25, 3389-3402

Bacher A., R. Ladenstein (1991) The lumazine synthase/riboflavin synthase complex of Bacillus subtilis in Chemistry and Biochemistry of Flavoproteins, Band 1 (F. Müller, ed.) CRC Press, Boca Raton, 293-316

Bacher A., H. C. Ludwig, H. Schnepple and Y. Ben-Shaul (1986) Heavy riboflavin synthase from Bacillus subtilis. Quaternary structure and reaggregation. J. Mol. Biol., 187, 75-86

Bacher A., H. Schnepple, B. Mailänder, M. K. Otto and Y. Ben-Shaul (1980) Structure and function of the riboflavin synthase complex of Bacillus subtilis in Flavins and Flavoproteins, (K. Yagi and T. Yamano, eds.) Japan Scientific Societies Press, 579-586

Bacher A., S. Eberhardt, M. Fischer, S. Mörtl, K. Kis, K. Kugelbrey, J. Scheuring and K. Schott (1997) Biosynthesis of riboflavin. Lumazine synthase and riboflavin synthase Methods Enzymol., 280, 389-399

Birnboim H. C., J. Doly (1979) A Rapid Alkaline Extraction Procedure for Screening Recombinant Plasmid DNA Nucl. Acids. Res., 7, 1513-1522

Bradford M. (1976) A rapid and selective method for the quantitation of microgramm quantities of protein utilizing the prinziple of protein-dye binding Anal. Biochem., 72, 248-254

Brigidi P., E. Rossi, M. Bertarini, G. Riccardi, D. Matteuzzi (1990) Genetic transformation of intact cells of Bacillus subtilis by electroporation FEMS Microbiology Letters, 67, 135-138

Bullock W. O., J. M. Fernandez, J. M. Short (1987) XL1-Blue: a high efficiency plasmid transforming recA Escherichia coli strain with β-Galactosidase selection BioTechniques, 5, 376-379

Clackson T., D. Güssow, P. T. Jones (1991) General applications of PCR to gene cloning and manipulation In: M. J. McPherson, P. Quirke, G. R. Taylor (eds.); PCR: A practical approach (187-214). Oxford Press

Cohn E., J. Edsall (1943) Proteins, amino acids and peptides as ions and dipolar ions Reinhold, N.Y.

Compton S. J., C. G. Jones (1985) Mechanism of Dye Response and Interference in the Bradford Protein Assay Annal. Biochem., 151, 369

Cronan J. E. (1990) Biotination of proteins in vivo The Journal of biological Chemistry, 265/18,10327-10333

Dalsgaard K., A. Uttenthal, T. Jones, F. Xu, A. Merryweather, W. Hamilton, J. Langeveld, R. Boshuizen, S. Kamstrup, G. Lomonossoff, C. Porta, C. Vela, J. Casal, R. Meloen, P. Rodgers (1997) Plant-derived vaccine protects target animals against a viral disease Nature Biotechnology, Volume 15, 248-252

Davis L. G., M. D. Dibner, J. F. Battey (1986) Basic Methods in molecular biology Elsevier, N.Y., Amsterdam, London

Deckert G., P. Warren, T. Gaasterland, W. Young, A. Lenox, D. Graham, R. Overbeek, M. Snead, M. Keller, M. Aujay, R. Huber, R. Feldman, J. Short, G. Olsen, R. Swanson (1998) The complete genome of the hyperthermophilic bacterium Aquifex aeolicus Nature, 392, 353-358

Dower W. J., J. F. Miller, C. W. Ragsdale (1988) High efficiency transformation of Escherichia coli by high voltage electroporation Nucl. Acids Res., 16, 6127-6145

Glick B. R., J. J. Pasternak (1995) Molekulare Biotechnologie Spektrum Akademischer Verlag, Heidelberg, Berlin, Oxford

Groseach an H., W. Fiers (1982) Preferential codon usage in procaryotic genes-the optimal codon-anticodon interaction energy and the selective codon usage in efficiently expressed genes Gene, 18, 199-209

Hennecke H., I. Günther, F. Binder (1982) A novel cloning vector for the direct selection of recombinant DNA in Escherichia coli Gene, 19, 231-234

Henner D. (1990) Expression of Heterologous Genes in Bacillus subtilis Meth. Enzymol., 185, 199

Ikemura T. (1981) Correlation between the abundance of Escherichia coli transfer RNAs and the occurence of the respective codons in ist protein genes: A proposal for a synonymous codon choice that is optimal for the Escherichia coli translational system J. Mol. Biol., 151, 389-409

Ladenstein R., B. Meyer, R. Huber, H. Labischinski, K. Bartels, H.-D. Bartunik, L. Bachmann, H. C. Ludwig and A. Bacher (1986) Heavy riboflavin synthase from Bacillus subtilis. Particle dimensions, crystal packing and molecular symmetry. J. Mol. Biol., 187, 87-100

Ladenstein R., H. D. Bartunik, M. Schneider, R. Huber, K. Schott and A. Bacher (1986) Structure of the riboflavin synthase/lumazine synthase complex. Arrangement and chain folding of 13 subunits in the icosahedral capsid. in Chemistry and Biology of Pteridines, (B. A. Cooper, V. M. Whitehead, eds.) Walter de Gruyter, Berlin, 103-106

Ladenstein R., K. Ritsert, R. Huber, G. Richter and A. Bacher (1994) The lumazine synthase/riboflavin synthase complex of Bacillus subtilis: X-ray structure analysis of reconstituted β₆₀ capsids at 3.2 Å resolution Eur. J. Biochem., 223, 1007-1017

Ladenstein R., M. Schneider, R. Huber, H. D. Bartunik, K. Wilson, K. Schott and A. Bacher (1988) Heavy riboflavin synthase from Bacillus subtilis. Crystal structure analysis of the icosahedral β₆₀ capsid at 3.3 Å resolution J. Mol. Biol., 203, 1045-1070

Ladenstein R., M. Schneider, R. Huber, K. Schott and A. Bacher (1988) The structure of the icosahedral β₆₀ capsid of heavy riboflavin synthase from Bacillus subtilis Z. Kristallographie, 185, 122-124

Laemmli U. K. (1970) Cleavage of Structural Proteins During the Assembly of the Head of Bacteriophage T4Nature, 227, 680-685

Laue T., B. Shah, T. Ridgeway, S. Pelletier (1992) Computer aided interpretation of analytical sedimentation data for proteins in: Harding S., Rowe A., Horton J. (eds.) Analytical ultracentrifugation in biochemistry and polymer science Royal society of chemistry, Cambridge, 90-125

LeGrice S. F. J. (1990) A regulated promotor for high level expression of heterologous genes in Bacillus subtilis Meth. Enzymol., 185, 201

Liddell E., I. Weeks (1996) Antikörpertechniken Spektrum Akademischer Verlag, Heidelberg, Berlin, Oxford

Lottspeich F., H. Zorbas (1998) Bioanalytik Spektrum Akademischer Verlag, Heidelberg, Berlin, Oxford

Lovett P. S. (1981) BR151ATCC 33677—Bacillus subtilis BR151 J. Bacteriol., 146, 1162-1165

Maniatis T., E. F. Fritsch, J. Sambrook (1982) Molecular cloning: A laboratory manual. Cold Spring Harbor Laboratory, Cold Spring Harbor, N.Y.

Modrow S., D. Falke (1997) Molekulare Virologie Spektrum Akademischer Veriag, Heidelberg, Berlin, Oxford

Mörtl S., M. Fischer, G. Richter, J. Tack, S. Weinkauf and A. Bacher (1996) Biosynthesis of riboflavin. Lumazine synthase of Escherichia coli. J. Biol. Chem., 271, 33201-33207

Mullis K., F. Faloona, S. Scharf, R. Saiki, G. Horn, H. Ehrlich (1986) Specific enzymatic amplification of DNA in vitro: The polymerase chain reaction Cold Spring Harbor Symp., 51, 263-273

Perkins J. B., J. G. Pero, A. Sloma (1991) Riboflavin overproducing bacteria expressing the rib-operon of Bacillus Eur. Pat. Appl. EP 405370 A1910102

Read S. M., D. H. Northcote (1981) Minimization of variation in the response to different proteins of the Coomassie Blue G dyebinding assay for protein Anal. Biochem., 116, 53-64

Sambrook J., E. Fritsch, T. Maniatis (1989) Molecular Cloning: A Laboratory Manual, 2nd ed. Cold Spring Harbor Laboratory Press, Cold Spring Harbor, N.Y.

Sanger F., S. Nicklen, A. R. Coulson (1977) DNA sequencing with chain terminating inhibitors Proc. Natl. Acad. Sci. USA, 74, 5463-5467

Schatz P. J. (1993) Use of peptide libraries to map the substrate specificity of a peptide-modifying enzyme: a 13 residue consensus peptide specifies biotinylation in Escherichia coli Bio/Technology, October; 11(10), 1138-1143

Sgamarella V., J. H. van de Sande, H. G. Khorana (1979) Studies on the polynukleotides. C. A novel joining reaction catalysed by the T4-polynukleotide-ligase Proc. Natl. Acad. Sci. USA, 67, 1468-1475

Stüber D., H. Matile, G. Garotta (1990) System for high-level production in Escherichia coli and rapid purification of recombinant proteins: Application to epitope mapping, preparation of antibodies and structure-function analysis in: Lefkovits, I., P. Pernis (eds.) Immunological Methods, IV, 121-152

Tucker J., Grisshammer (1996) Purification of a rat neurotensin receptor expressed in Escherichia coli Biochem. Journal, 317, 891-899

Wada K., Y. Wada, F. Ishibashi, T. Gojobori, T. Ikemura (1992) Codon usage tabulated from the GenBank genetic sequence data. Nucleic Acid Res., 20, 2111-2118

Winnacker E. L. (1990) Gene und Klone. Eine Einführung in die Gentechnologie Verlag Chemie, Weinheim, Dearfield Beach, Basel

Zamenhof P. J., M. Villarejo (1972) Construction and properties of Escherichia coli strains exhibiting α-complementation of β-galactosidase fragments in vivo J. Bacteriol., 110, 171-178

1. Protein conjugate consisting of at least one functional region in an arbitrary position of the sequence of a carrier protein for formation of a capsid-type spatial structure of the lumazine synthase type, whereby the outer periphery thereof is covalently linked with a multiple number of the functional regions.

2. Protein conjugate which can be produced by recombinant technology and which consists of at least one functional protein region at the N-terminus and/or C-terminus and/or inserted into a loop region of the sequence of a carrier protein region for formation of a capsid-type spatial structure of the lumazine synthase type, whereby the outer periphery thereof is covalently linked with a multiple number of the functional regions.

3. Protein conjugate according to claim 1 and 2, whereby the carrier protein region comprises an amino acid sequence—selected from a set of sequences—which is obtained by a procedure whereby for every amino acid position in the sequence of a predetermined native lumazine synthase, an amino acid or a deletion is selected from the respective position of an alignment of the predetermined lumazine synthase sequence with at least one native lumazine synthase sequence of another organism.

4. Protein conjugate according to claim 1 and 2, whereby the carrier protein region has the sequence of a native lumazine synthase.

5. Protein conjugate according to claim 1 and 2, whereby the carrier protein region has the sequence of a thermostable native lumazine synthase.

6. Protein conjugate according to claim 1 and 2 whereby the thermostable native lumazine synthase has the protein sequence of the lumazine synthase of a hyperthermophilic microorganism, preferentially Aquifex aeolicus.

7. Protein conjugate according to claim 1 and 2 whereby the carrier protein region consists of a mixed sequence comprising amino acid positions 1-60 of the native lumazine synthase of a mesophilic organism referenced to Bacillus subtilis, and the amino acid positions 61-154 of the native lumazine synthase of a hyperthermophilic microorganism referenced to Aquifex aeolicus.

8. Protein conjugate according to claim 1 and 2, whereby the carrier protein region consists of an arbitrary sequence, whereby the main chain of the sequence folds into α-helix and β-pleated sheet motifs, whereby 4 β-segments form a parallel 4-stranded β-pleated sheet which is flanked on both sides by two respective α-helices, whereby 5 units of these α-β-motifs associate under formation of a pentameric structure, whereby the N-terminus of each unit can form the fifth β-segment to the central 4-stranded β-pleated sheet of the adjacent unit, whereby 12 of these pentameric substructures associate under formation of the icosahedral structure of a lumazine synthase and whereby the N- and C-termini of the arbitrary sequence with the structural characteristics described above are located at the surface of the hereby formed icosahedron and whereby the arbitrary sequence is preferentially obtained by the comparison of a set of sequences of different lumazine synthase sequences, i.e. lumazine synthase sequences derived from lumazine synthase genes of different organisms, in particular by search algorithms according to Altschul et al. (1997).

9. Protein conjugate according to claim 1 and 2 whereby the carrier protein region comprises a sequence of a native lumazine synthase whereby at least one cystein unit is replaced by another amino acid or is deleted or is chemically modified.

10. Protein conjugate according to claim 9 whereby a cystein unit in a position corresponding to one of the positions 93 and/or position 139 of the lumazine synthase of Bacillus subtilis is deleted or is replaced by another aminoacid, preferentially serine.

11. Protein conjugate according to one of the claims 1 to 10, whereby the carrier protein region and the functional protein region are linked by a linker peptide.

12. Protein conjugate according to one of the claims 2 to 11, whereby the functional protein region is the sequence of a dihydrofolate reductase, a maltose binding protein, a protein that is susceptible to in vivo biotinylation, an antigenically active peptide, especially from a surface protein of a virus, a peptide that can be recognized by a monoclonal antibody, a stochastically generated peptide or an amino acid that is susceptible to chemical derivatization, e.g. cystein or lysin.

13. Protein conjugate according to one of the claims 2 to 12, whereby the carrier protein region is chemically modified.

14. Protein conjugate according to one of the claims 2 to 13, whereby the functional protein region is chemically modified, preferably biotinylated.

15. Heterooligomeric protein conjugate consisting of mixtures of at least two different protein conjugates according to one of the claims 1 to 14 or at least one protein conjugate according to one of the claims 1 to 14 and at least one carrier protein region without functional protein region with a sequence according to one of the claims 3 to 8, whereby the individual proteins are covalently coupled by chemical treatment if required.

16. Procedure for preparation of a protein conjugate according to claim 1 characterized by the following steps,
a) isolation of a lumazine synthase from a wild type or a recombinant organism (carrier protein);
b) chemical coupling of functional molecules to the carrier protein.
c) purification of the protein conjugate.

17. Procedure for preparation of a protein conjugate or a heterooligomeric protein according to one of the claims 2 to 14 which is characterized by the following steps,
a) Preparation of a first DNA coding for the carrier protein region
b) Fusion of at least one second DNA coding for the functional region and for the linker protein, if required, at the 5′ end and/or the 3′ end of the first DNA and/or insertion of the second DNA into a region of the first DNA coding for a loop region of the carrier protein under formation of an artificial DNA.
c) Conversion of the artificial DNA of step b) into an expression plasmid.
d) Transformation of host cells with one or several of the expression plasmids generated in step c).
e) Expression of the artificial DNA in the transformed host cells under formation of a protein conjugate, if required under introduction of a predetermined post-translational modification of the protein conjugate in vivo, preferably by phosphorylation, glycosidation or biotinylation.
f) Purification of the protein conjugate.
g) Modification of the protein conjugate, if required, by chemical coupling of amino acid residues on the protein surface of a capsid-type spatial structure formed from the protein conjugate with arbitrarily determined coupling partners.

18. Procedure according to claim 15 characterized by the production of a heterooligomeric protein by
a) mixing of different protein conjugates obtained according to claim 16, step c) and/or claim 17, step f)
b) denaturation of the resulting mixture and
c) renaturation of the mixture; or by
a₂) denaturation of different protein conjugates obtained according to claim 16, step c) and/or claim 17, step f)
b₂) mixing the denatured protein conjugate
c₂) renaturation of the mixture

19. Procedure according to claim 15, characterized by the use of protein conjugates which were produced with the use of a ligand which supports the folding

20. Vectors for preparation of the protein conjugates according to one of the claims of 2 to 14.

21. DNA coding for a protein according to claim 20.

22. Protein consisting of the lumazine synthase of Bacillus subtilis, whereby the amino acid cystein in position 93 is replaced by the amino acid serine.

23. Protein consisting of the lumazine synthase of Bacillus subtilis whereby the amino acid cystein in position 139 is replaced by the amino acid serine.

24. Protein, consisting of the lumazine synthase of Bacillus subtilis whereby the amino acid cystein in the positions 93 and 139 is replaced by the amino acid serine.

25. DNA adapted to the codon usage of Escherichia coli for preparation of the lumazine synthase of Aquifex aeolicus in a recombinant Escherichia coli strain.

26. Protein consisting of the lumazine synthase of Aquifex aeolicus for use as carrier protein according to claim 1.

27. Chimeric protein consisting of the amino acids 1-60 of the lumazine synthase of Bacillus subtilis and the amino acids 61-154 of the lumazine synthase of Aquifex aeolicus for use as carrier protein according to claim 1.

28. Vector for preparation of protein conjugates according to claim 12, whereby the functional DNA part is located at the 5′ end of the carrier protein gene of the lumazine synthase type, whereby the fused gene codes for an artificial protein which contains a functional protein region, a carrier protein region for formation of a capsid-type spatial structure of the lumazine synthase type and optionally a linker peptide, and whereby the functional protein region and the linker peptide are located at the N-terminus of the carrier protein region and whereby the vector contains the following components:
a) a DNA fragment coding for a carrier protein region for formation of a capsid type spatial structure of the lumazine synthase type
b) a DNA fragment coding for an arbitrarily selected functional protein region.
c) optional: a DNA fragment coding for a linker peptide.

29. A vector according to claim 28 whereby it contains the gene for the lumazine synthase of Bacillus subtilis coding for the carrier protein region, the gene for the dihydrofolate reductase of Escherichia coli coding for the functional protein region and, as linker peptide, a DNA fragment coding for a tripeptide consisting of the amino acid alanine.

30. A vector according to claim 28 whereby it contains the gene for the lumazine synthase of Bacillus subtilis coding for the carrier protein region, the gene for the “maltose binding protein” of Escherichia coli coding for the functional protein region and as linker peptide a DNA fragment coding for the amino acid sequence SNNNNNNNNNNLGIEGRISEFAAA.

31. Vector for preparation of protein conjugates according to claim 12, whereby the functional DNA part is located at the 3′ end of the carrier protein gene of the lumazine synthase type and whereby the fused gene codes for an artificial protein which contains a functional protein region, a carrier protein region for formation of a capsid-type spatial structure of the lumazine synthase type, and optionally a linker peptide, and whereby the functional protein region and the linker peptide are located at the C-terminus of the carrier protein region and whereby the vector contains the following components:
a) a DNA fragment coding for a carrier protein region for formation of a capsid-type spatial structure of the lumazine synthase type (without respective stop codon)
b) a DNA fragment coding for an arbitrarily selected functional protein region
c) optional: a DNA fragment coding for a linker peptide.

32. Vector according to claim 31 whereby it contains the gene for the lumazine synthase of Bacillus subtilis coding for the carrier protein region, the gene for dihydrofolate reductase of Escherichia coli coding for the functional protein region and as linker peptide a DNA fragment coding for the amino acid sequence LAAAGGGG.

33. Vector according to claim 31 whereby it contains the gene for the lumazine synthase of Aquifex aeolicus (according to claim 25) coding for the carrier protein region and a gene fragment coding for the functional protein region with the amino acid sequence GSVDLQPSLIS. The vector comprises a singular recognition sequence at the 5′ end of the gene sequence of the carrier protein for the restriction endonucleases BglII, whereby this restriction site can be used for the fusion of foreign genes to the 5′ end of the lumazine synthase.

34. Vector for preparation of protein conjugates according to claim 12, whereby the functional DNA part is located at the 3′ end of the carrier protein gene of the lumazine synthase type and whereby the fused gene codes for an artificial protein which contains a functional protein region, a carrier protein region for formation of a capsid-type spatial structure of the lumazine synthase type, and optinally a linker peptide, and whereby the functional protein region and the linker peptide are located at the C-terminus of the carrier protein region and whereby the selected functional protein region is biotinylated in vivo and whereby the vector contains the following components:
a) a DNA fragment coding for a carrier protein region for formation of a capsid-type spatial structure of the lumazine synthase type (without respective stop codon)
b) a DNA fragment coding for a peptide susceptible to biotinylation with the sequence LGGIFEAMKMEWR, whereby the amino acid lysin is biotinylated in vivo
c) optional: a DNA fragment coding for a linker peptide.

35. A vector according to claim 34 whereby it contains the gene for the lumazine synthase of Bacillus subtilis coding for the carrier protein region and as linker peptide a DNA fragment coding for a tripeptide consisting of the amino acid alanine.

36. A vector according to claim 34 whereby it contains the gene, adapted to the codon usage of Escherichia coli, coding for the lumazine synthase of Aquifex aeolicus according to claim 25 as carrier protein region and as linker peptide a DNA fragment coding for a tripeptide consisting of the amino acid alanine.

37. A vector according to claim 34 whereby it contains the gene, adapted to the codon usage of Escherichia coli, coding for the lumazine synthase of Aquifex aeolicus according to claim 25 as carrier protein region and as linker peptide a DNA fragment coding for a peptide consisting of the amino acid sequence HHHAAA.

38. A vector according to claim 34 whereby it contains the gene adapted to the codon usage of Escherichia coli coding for the lumazine synthase of Aquifex aeolicus according to claim 25 as carrier protein region and as linker peptide a DNA fragment coding for a peptide consisting of the amino acid sequence HHHHHHGGSGAAA.

39. Vector for production of a protein conjugate according to claim 12 whereby the functional DNA part is located at the 5′ end of the lumazine synthase gene of Bacillus subtilis, whereby the functional DNA part codes for an antigenically active peptide of the VP2 surface protein of the “mink enteritis virus” and the fused gene codes for an artificial protein comprising a functional protein part and a carrier protein part and whereby the functional protein part is located at the N-terminus of the lumazine synthase and whereby the vector has the following components:
a) Lumazine synthase gene of Bacillus subtilis.
b) DNA at the 5′ end of the lumazine synthase gene coding for peptide. The foreign peptide has the sequence MGDGAVQPDGGQPAVRNER.

40. Vector for production of a protein conjugate according to claim 12 whereby the functional DNA part is located at the 3′ end of the lumazine synthase gene of Bacillus subtilis, whereby the functional DNA part codes for an antigenically active peptide from the VP2 surface protein of the “mink enteritis virus”, and whereby the fused gene codes for an artificial protein comprising a functional protein part and a carrier protein part, whereby the functional protein part is located at the C-terminus of the lumazine synthase and whereby the vector contains the following components:
a) Lumazine synthase gene from Bacillus subtilis (without stop codon).
b) DNA coding for peptide at the 3′ end of the lumazine synthase gene. The foreign peptide has the sequence GDGAVQPDGGQPAVRNER.

41. Vector for production of a protein conjugate according to claim 12 whereby the functional DNA part is located at the 5′ end and at the 3′ end of the lumazine synthase gene of Bacillus subtilis, and whereby the functional DNA part codes for an antigenically active peptide from the VP2 surface protein of the “mink enteritis virus”, and whereby the fused gene codes for an artificial protein comprising a functional protein part and a carrier protein part, and whereby the functional protein part is located at the N-terminus as well as at the C-terminus of the lumazine synthase and whereby the vector contains the following components:
a) Lumazine synthase gene from Bacillus subtilis (without stop codon).
b) Two sequences coding for peptides at the 5′ and the 3′ end of the lumazine synthase gene. The peptide at the N-terminus has the sequence MGDGAVQPDGGQPAVRNER, the peptide at the C-terminus has the sequence GDGAVQPDGGQPAVRNER.

42. Vector for production of a protein conjugate according to claim 12 whereby the functional DNA region is located at the 5′ end of the lumazine synthase gene from Bacillus subtilis and the functional DNA part codes for an octapeptide (FLAG peptide) which is recognized by a monoclonal antibody (preferentially Anti-FLAG-M2; IBI E. coli FLAG® Expression System, Integra Biosciences, Fernwald), whereby the fused gene codes for an artificial protein which contains a functional protein region and a carrier protein region and whereby the functional protein region is located at the N-terminus of the lumazine synthase and whereby the vector comprises the following components:
a) Lumazine synthase gene from Bacillus subtilis
b) DNA coding for peptide at the 5′ end of the lumazine synthase gene. The foreign peptide has the sequence MDYKDDDDK;
c) DNA coding for a linker peptide with the sequence VKL

43. Vector for production of a protein conjugate according to claim 12 whereby the functional DNA region is located at the 3′ end of the lumazine synthase of Bacillus subtilis, whereby the functional DNA part codes for a hexapeptide (His6-peptide) which is recognized by a monoclonal antibody (preferentially Penta-His™ antibody; Qiagen, Hilden) and whereby the fused gene codes for an artificial protein comprising a functional protein region and a carrier protein region and whereby the functional protein region is located at the C-terminus of the lumazine synthase and whereby the vector contains the following components:
a) Lumazine synthase gene from Bacillus subtilis without stop codon.
b) DNA coding for peptide at the 3′ end of the lumazine synthase gene. The foreign peptide has the sequence HHHHHH.

44. Vector for production of a protein conjugate according to claim 12 whereby the functional DNA region is located at the 3′ and of the lumazine synthase gene from Bacillus subtilis, whereby the functional DNA region codes for an artificial peptide sequence which ends with the amino acid lysin, whereby the amino acid lysin can be used for chemical coupling of functional molecules to the carrier protein region (cf. claim 16 b) and whereby the functional protein region serves as linker (tentacle-linker) and whereby the fused gene codes for an artificial protein comprising a functional protein region and a carrier protein region, whereby the functional protein region is located at the C-terminus of the carrier protein region and whereby the vector comprises the following components:
a) Lumazine synthase gene from Bacillus subtilis (without stop codon).
b) Codon for lysin (aaa) at the 3′ end of the artificial DNA.
c) DNA coding for a linker peptide with the sequence GGGGSGGGSG.

45. Vector for production of a protein conjugate according to claim 12 whereby the functional DNA region is located at the 3′ end of the lumazine synthase gene from Bacillus subtilis, whereby the functional DNA region codes for an artificial peptide sequence which ends with the amino acid cystein, whereby the amino acid cystein can be used for chemical coupling of fusion molecules to the carrier protein region (cf. claim 16 b), and whereby the functional protein region serves as linker (tentacle linker) and whereby the fused gene codes for an artificial protein which comprises a functional protein region and a carrier protein region, whereby the functional protein region is located at the C-terminus of the carrier protein region and whereby the vector comprises the following components:
a) Lumazine synthase gene from Bacillus subtilis (without stop codon).
b) Codon for cystein (tgc) at the 3′ end of the artificial DNA.
c) DNA coding for a linker peptide with the sequence GGGGSGGGSGGG.

46. Protein conjugates according to one of the claims 1 to 15 for preparation of a medicament (pharmacological agent) or a vaccine.

47. Use of the protein conjugates according to one of the claims 1 to 15 for preparation of a medicament (pharmacological agent) or a vaccine.

48. Use of the protein conjugates according to one of the claims 1 to 15 for preparation of diagnostically or therapeutically applicable antibodies

49. Use of the protein conjugates according to one of the claims 1 to 15 for selective detection of antibodies or for purification of antibody mixtures or for characterization of antibodies.

50. Use of protein conjugates according to one of the claims 1 to 15 for preparation of protein libraries

51. Medicaments (pharmacological agents) containing a pharmacologically active quantity of a protein conjugate according to one of the claims 1 to 15

52. Vaccine containing an immunologically active quantity of a protein conjugate according to one of the claims 1 to 15

53. Use of protein conjugates according to one of the claims 1 to 15 as biosensor.


SEQUENCE LISTING (154 sequences)

SEQ ID NO: 1 - DNA, 3420 bp, Artificial sequence
  pNCO113 Expression vector
ctcgagaaatcataaaaaatttatttgctttgtgagcggataacaattataatagattcaattgtgagcggataacaatttcacacagaattcattaaagaggagaaattaaccatgggaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 2 - DNA, 5302 bp, Artificial sequence
  p6021-CAT Expression vector
gaattaattcctcgaggctggcatccctaacatatccgaatggttacttaaacaacggaggactagcgtatcccttcgcatagggtttgagttagataaagtatatgctgaactttcttctttgctcaaagaatcataaaaaatttatttgctttcaggaaaatttttctgtataatagattcaaattgtgagcggataacaatttgaattcattaaagaggagaaattaactatgaggggatccgtcgacctgcagccaagcttagctagctagagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctgccgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcggtaccgctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgtcctgcgaggcgtccagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagcttcatcacttgcctcccatagatgaatccgaacctcattacacattagaactgcgaatccatcttcatggtgaaccaaagtgaaacctagtttatcgcaataaaaacctatactctttttaatatccccgactggcaatgcgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgtaaccaacatgattaacaattattagaggtcatcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgaagcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagttggatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaag

SEQ ID NO: 3 - DNA, 3885 bp, Artificial sequence
  pNCO-BS-LuSy Expression vector
ctcgagaaatcataaaaaatttatttgctttgtgagcggataacaattataatagattcaattgtgagcggataacaatttcacacagaattcattaaagaggagaaattaaccatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaataaccatggggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 4 - DNA, 5767 bp, Artificial sequence
  p602-BS-LuSy Expression plasmid
gaattaattcctcgaggctggcatccctaacatatccgaatggttacttaaacaacggaggactagcgtatcccttcgcatagggtttgagttagataaagtatatgctgaactttcttctttgctcaaagaatcataaaaaatttatttgctttcaggaaaatttttctgtataatagattcaaattgtgagcggataacaatttgaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaataaccatggggatccgtcgacctgcagccaagcttagctagctagagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctgccgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcggtaccgctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgtcctgcgaggcgtccagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagcttcatcacttgcctcccatagatgaatccgaacctcattacacattagaactgcgaatccatcttcatggtgaaccaaagtgaaacctagtttatcgcaataaaaacctatactctttttaatatccccgactggcaatgcgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgtaaccaacatgattaacaattattagaggtcatcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgaagcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagttggatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaag

SEQ ID NO: 5 - DNA, 3879 bp, Artificial sequence
  pNCO-N-BS-LuSy-C93S Expression plasmid
ctcgagaaatcataaaaaatttatttgctttgtgagcggataacaattataatagattcaattgtgagcggataacaatttcacacagaattcattaaagaggagaaattaaccatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtttcgaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 6 - DNA, 3879 bp, Artificial sequence
  pNCO-C-BS-LuSy C139S Expression plasmid
ctcgagaaatcataaaaaatttatttgctttgtgagcggataacaattataatagattcaattgtgagcggataacaatttcacacagaattcattaaagaggagaaattaaccatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattcagctgtttctgccattgaaatggcaaatttaaaccgctcatttgaataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 7 - DNA, 3879 bp, Artificial sequence
  pNCO-BS-LuSy C93/139S Expression plasmid
ctcgagaaatcataaaaaatttatttgctttgtgagcggataacaattataatagattcaattgtgagcggataacaatttcacacagaattcattaaagaggagaaattaaccatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtttcgaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattcagctgtttctgccattgaaatggcaaatttaaaccgctcatttgaataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 8 - DNA, 3912 bp, Artificial sequence
  pNCO-EC-N-BS-LuSy Expression vector
ctcgagaaatcataaaaaatttatttgctttgtgagcggataacaattataatagattcaattgtgagcggataacaatttcacacagaattcattaaagaggagaaattaactatggcggcggcgcgtagctgcgcggccgctatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataatttggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 9 - DNA, 3900 bp, Artificial sequence
  pNCO-C-BS-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaattagcggccgcaaacagtttaaaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 10 - DNA, 4368 bp, Artificial sequence
  pNCO-EC-DHFR-BS-LuSy Expression vector
ctcgagaaatcataaaaaatttatttgctttgtgagcggataacaattataatagattcaattgtgagcggataacaatttcacacagaattgattaaagaggagaaattaactatgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgtgcggccgctatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataatttggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 11 - DNA, 5064 bp, Artificial sequence
  pNCO-EC-MBP-BS-LuSy Expression vector
ctcgagaaatcataaaaaatttatttgctttgtgagcggataacaattataatagattcaattgtgagcggataacaatttcacacagaattgattaaagaggagaaattaactatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgcggccgctatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataatttggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 12 - DNA, 4380 bp, Artificial sequence
  pNCO-BS-LuSy-EC-DHFR Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaattagcggccgctggtggaggcggaatgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 13 - DNA, 3936 bp, Artificial sequence
  pNCO-N-VP2-BS-LuSy Expression vector
ctcgagaaatcataaaaaatttatttgctttgtgagcggataacaattataatagattcaattgtgagcggataacaatttcacacagaattcattaaagaggagaaattaactatgggggacggtgctgttcagccggacggtggtcagccggctgttcgtaacgaacgtatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 14 - DNA, 3932 bp, Artificial sequence
  pNCO-C-VP2-BS-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaaggtgacggtgctgttcagccggacggtggtcagccggctgttcgtaacgaacgttaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 15 - DNA, 3989 bp, Artificial sequence
  pNCO-N/C-VP2-BS-LuSy Expression vector
ctcgagaaatcataaaaaatttatttgctttgtgagcggataacaattataatagattcaattgtgagcggataacaatttcacacagaattcattaaagaggagaaattaactatgggggacggtgctgttcagccggacggtggtcagccggctgttcgtaacgaacgtatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaaggtgacggtgctgttcagccggacggtggtcagccggctgttcgtaacgaacgttaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 16 - DNA, 3927 bp, Artificial sequence
  pNCO-C-Biotag-BS-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaagcggccgcactcggcggcatcttcgaagctatgaagatggagtggcgctaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 17 - DNA, 3912 bp, Artificial sequence
  pNCO-Lys165-BS-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaaggtggcggtggttctggtggtggctctggtaaataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 18 - DNA, 3919 bp, Artificial sequence
  pNCO-Cys167-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaaggtggcggtggttctggtggtggctctggtggtggctgctaaggatcccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 19 - DNA, 5531 bp, Artificial sequence
  pFLAG-MAC-BS-LuSy Expression vector
catcataacggttctggcaaatattctgaaatgagctgttgacaattaatcatcggctcgtataatgtgtggaattgtgagcggataacaatttcacacaggagatatcttatggactacaaggacgacgatgacaaagtcaagcttatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaataagaattcccgggtacctgcagatctagatagatgagctcgtcgagtgagagaagattttcagcctgatacagattaaatcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccatgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagatcctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaggatctaatgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattccatggtgtcgaattgctgcaggtcgagggggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacgggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccga

SEQ ID NO: 20 - DNA, 3897 bp, Artificial sequence
  pNCO-His6-BS-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaacttaaaccgttctttcgaacatcaccatcaccaccattaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 21 - DNA, 3879 bp, Artificial sequence
  pNCO-AA-LuSy Expression vector
ctcgagaaatcataaaaaatttatttgctttgtgagcggataacaattataatagattcaattgtgagcggataacaatttcacacagaattcattaaagaggagaaattaactatgcaaatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 22 - DNA, 3927 bp, Artificial sequence
  pNCO-C-Biotag-AA-LuSy Expression vector
ctcgagaaatcataaaaaatttatttgctttgtgagcggataacaattataatagattcaattgtgagcggataacaatttcacacagaattcattaaagaggagaaattaactatgcaaatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgagcggccgcactcggcggcatcttcgaagctatgaagatggagtggcgctaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 23 - DNA, 3945 bp, Artificial sequence
  pNCO-His6-C-Biotag-AA-LuSy Expression vector
ctcgagaaatcataaaaaatttatttgctttgtgagcggataacaattataatagattcaattgtgagcggataacaatttcacacagaattcattaaagaggagaaattaactatgcaaatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgacatcaccatcaccaccatgcggccgcactcggcggcatcttcgaagctatgaagatggagtggcgctaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 24 - DNA, 3957 bp, Artificial sequence
  pNCO-His6-Gly2-Ser-Gly-C- Biotag-AA-LuSy Expression vector
ctcgagaaatcataaaaaatttatttgctttgtgagcggataacaattataatagattcaattgtgagcggataacaatttcacacagaattcattaaagaggagaaattaactatgcaaatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgacatcaccatcaccaccatggcggttctggcgcggccgcactcggcggcatcttcgaagctatgaagatggagtggcgctaaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 25 - DNA, 3879 bp, Artificial sequence
  pNCO-BS-LuSy-AgeI-AA-LuSy Expression vector
ctcgagaaatcataaaaaatttatttgctttgtgagcggataacaattataatagattcaattgtgagcggataacaatttcacacagaattcattaaagaggagaaattaactatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 26 - DNA, 3879 bp, Artificial sequence
  pNCO-AA-BglII-LuSy Expression vector
ctcgagaaatcataaaaaatttatttgctttgtgagcggataacaattataatagattcaattgtgagcggataacaatttcacacagaattcattaaagaggagaaattaactatgcagatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgataaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 27 - DNA, 3876 bp, Artificial sequence
  pNCO-AA-LuSy-(BamHI) Expression vector
ctcgagaaatcataaaaaatttatttgctttgtgagcggataacaattataatagattcaattgtgagcggataacaatttcacacagaattcattaaagaggagaaattaactatgcaaatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 28 - DNA, 3876 bp, Artificial sequence
  pNCO-AA-BglII-LuSy-(BamHI) Expression vector
ctcgagaaatcataaaaaatttatttgctttgtgagcggataacaattataatagattcaattgtgagcggataacaatttcacacagaattcattaaagaggagaaattaactatgcagatctacgaaggtaaactaactgctgaaggccttcgtttcggtatcgtagcatcacgttttaatcatgctcttgtcgaccgtctggtggagggtgcaattgattgcatagtccgtcatggcggccgtgaagaagacattactctggttcgtgttccaggctcatgggaaataccggttgctgcgggtgaactggcgcgtaaagaggacattgatgctgttatcgcaattggcgttctcatcagaggcgcaacgccacatttcgattatatcgcctctgaagtttcaaaaggcctcgcgaacctttcattagaactacgtaaacctatcaccttcggtgttattacagctgacaccttggaacaggctatcgagcgcgccggcacaaaacacggcaacaaaggttgggaagcagcgctttctgccattgaaatggcaaacttattcaagtctctccgaggatccgtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 29 - DNA, 39 bp, Artificial sequence
  Synthetic oligonucleotide primer
gaggagaaattaaccatgaatatcatacaaggaaattta

SEQ ID NO: 30 - DNA, 44 bp, Artificial sequence
  Synthetic oligonucleotide primer
tattatggatccccatggttattcgaaagaacggtttaagtttg

SEQ ID NO: 31 - DNA, 36 bp, Artificial sequence
  Synthetic oligonucleotide primer
ataatagaattcattaaagaggagaaattaaccatg

SEQ ID NO: 32 - DNA, 25 bp, Artificial sequence
  Synthetic oligonucleotide primer
gtgagcggataacaatttcacacag

SEQ ID NO: 33 - DNA, 36 bp, Artificial sequence
  Synthetic oligonucleotide primer
ataatagaattcattaaagaggagaaattaactatg

SEQ ID NO: 34 - DNA, 26 bp, Artificial sequence
  Synthetic oligonucleotide primer
gtataatagattcaaattgtgagcgg

SEQ ID NO: 35 - DNA, 24 bp, Artificial sequence
  Synthetic oligonucleotide primer
agatattttcattaaagaggagaa

SEQ ID NO: 36 - DNA, 38 bp, Artificial sequence
  Synthetic oligonucleotide primer
tattatggatccttattcaaatgagcggtttaaatttg

SEQ ID NO: 37 - DNA, 29 bp, Artificial sequence
  Synthetic oligonucleotide primer
gcagcttcattcgaaacataatcgtaatg

SEQ ID NO: 38 - DNA, 31 bp, Artificial sequence
  Synthetic oligonucleotide primer
ggcagaaacagctgaatctacacctttgttg

SEQ ID NO: 39 - DNA, 58 bp, Artificial sequence
  Synthetic oligonucleotide primer
actatggcggcggcgcgtagctgcgcggccgctatgaatatcatacaaggaaatttag

SEQ ID NO: 40 - DNA, 41 bp, Artificial sequence
  Synthetic oligonucleotide primer
tattatggatccaaattattcaaatgagcggtttaaatttg

SEQ ID NO: 41 - DNA, 54 bp, Artificial sequence
  Synthetic oligonucleotide primer
ataatagaattcattaaagaggagaaattaactatggcggcggcgcgtagctgc

SEQ ID NO: 42 - DNA, 59 bp, Artificial sequence
  Synthetic oligonucleotide primer
ttttcgggatccttttaaactgtttgcggccgctaattcaaatgagcggtttaaatttg

SEQ ID NO: 43 - DNA, 34 bp, Artificial sequence
  Synthetic oligonucleotide primer
gaggagaaattaactatgatcagtctgattgcgg

SEQ ID NO: 44 - DNA, 42 bp, Artificial sequence
  Synthetic oligonucleotide primer
ctagccgtaaattctatagcggccgcacgccgctccagaatc

SEQ ID NO: 45 - DNA, 36 bp, Artificial sequence
  Synthetic oligonucleotide primer
ataatacaattgattaaagaggagaaattaactatg

SEQ ID NO: 46 - DNA, 37 bp, Artificial sequence
  Synthetic oligonucleotide primer
gaggagaaattaactatgaaaatcgaagaaggtaaac

SEQ ID NO: 47 - DNA, 30 bp, Artificial sequence
  Synthetic oligonucleotide primer
gcaggtcgactctagcggccgcgaattctg

SEQ ID NO: 48 - DNA, 56 bp, Artificial sequence
  Synthetic oligonucleotide primer
atagtggcgacaatgcggccgctggtggaggcggaatgatcagtctgattgcggcg

SEQ ID NO: 49 - DNA, 31 bp, Artificial sequence
  Synthetic oligonucleotide primer
ttctatggatccttaccgccgctccagaatc

SEQ ID NO: 50 - DNA, 59 bp, Artificial sequence
  Synthetic oligonucleotide primer
ggtcagccggctgttcgtaacgaacgtatgaatatcatacaaggaaatttagttggtac

SEQ ID NO: 51 - DNA, 71 bp, Artificial sequence
  Synthetic oligonucleotide primer
gaggagaaattaactatgggggacggtgctgttcagccggacggtggtcagccggctgttcgtaacgaacg

SEQ ID NO: 52 - DNA, 54 bp, Artificial sequence
  Synthetic oligonucleotide primer
ccaccgtccggctgaacagcaccgtcaccttcgaaagaacggtttaagtttgcc

SEQ ID NO: 53 - DNA, 65 bp, Artificial sequence
  Synthetic oligonucleotide primer
atatatggatcctaacgttcgttacgaacagccggctgaccaccgtccggctgaacagcaccgtc

SEQ ID NO: 54 - DNA, 63 bp, Artificial sequence
  Synthetic oligonucleotide primer
catagcttcgaagatgccgccgagtgcggccgcttcgaaagaacggtttaagtttgccatttc

SEQ ID NO: 55 - DNA, 60 bp, Artificial sequence
  Synthetic oligonucleotide primer
tattatggatccttagcgccactccatcttcatagcttcgaagatgccgccgagtgcggc

SEQ ID NO: 56 - DNA, 78 bp, Artificial sequence
  Synthetic oligonucleotide primer
tattatggatccttatttaccagagccaccaccagaaccaccgccaccttcgaaagaacggtttaagtttgccatttc

SEQ ID NO: 57 - protein, 11 aa, Artificial sequence
  Synthetic peptide sequence
GGGGSGGGSGK

SEQ ID NO: 58 - DNA, 84 bp, Artificial sequence
  Synthetic oligonucleotide primer
tattatggatccttagcagccaccaccagagccaccaccagaaccaccgccaccttcgaaagaacggtttaagtttgccatttc

SEQ ID NO: 59 - protein, 13 aa, Artificial sequence
  Synthetic peptide sequence
GGGGSGGGSGGGC

SEQ ID NO: 60 - DNA, 40 bp, Artificial sequence
  Synthetic oligonucleotide primer
ataataataaagcttatgaatatcatacaaggaaatttag

SEQ ID NO: 61 - DNA, 34 bp, Artificial sequence
  Synthetic oligonucleotide primer
tattatgaattcttattcgaaagaacggtttaag

SEQ ID NO: 62 - DNA, 34 bp, Artificial sequence
  Synthetic oligonucleotide primer
gtggtgatggtgatgttcgaaagaacggtttaag

SEQ ID NO: 63 - DNA, 33 bp, Artificial sequence
  Synthetic oligonucleotide primer
tattatggatccttaatggtggtgatggtgatg

SEQ ID NO: 64 - DNA, 63 bp, Artificial sequence
  Synthetic oligonucleotide primer
gctgcgggtgaactggcgcgtaaagaggacattgatgctgttatcgcaattggcgttctcatc

SEQ ID NO: 65 - DNA, 63 bp, Artificial sequence
  Synthetic oligonucleotide primer
ctaatgaaaggttcgcgaggccttttgaaacttcagaggcgatataatcgaaatgtggcgttg

SEQ ID NO: 66 - DNA, 64 bp, Artificial sequence
  Synthetic oligonucleotide primer
actctggttcgtgttccaggctcatgggaaataccggttgctgcgggtgaactggcgcgtaaag

SEQ ID NO: 67 - DNA, 70 bp, Artificial sequence
  Synthetic oligonucleotide primer
ccaaggtgtcagctgtaataacaccgaaggtgataggtttacgtagttctaatgaaaggttcgcgaggcc

SEQ ID NO: 68 - DNA, 73 bp, Artificial sequence
  Synthetic oligonucleotide primer
ggagggtgcaattgattgcatagtccgtcatggcggccgtgaagaagacattactctggttcgtgttccaggc

SEQ ID NO: 69 - DNA, 60 bp, Artificial sequence
  Synthetic oligonucleotide primer
gttgccgtgttttgtgccggcgcgctcgatagcctgttccaaggtgtcagctgtaataac

SEQ ID NO: 70 - DNA, 71 bp, Artificial sequence
  Synthetic oligonucleotide primer
cggtatcgtagcatcacgttttaatcatgctcttgtcgaccgtctggtggagggtgcaattgattgcatag

SEQ ID NO: 71 - DNA, 69 bp, Artificial sequence
  Synthetic oligonucleotide primer
gaataagtttgccatttcaatggcagaaagcgctgcttcccaacctttgttgccgtgttttgtgccggc

SEQ ID NO: 72 - DNA, 70 bp, Artificial sequence
  Synthetic oligonucleotide primer
atgcaaatctacgaaggtaaactaactgctgaaggccttcgtttcggtatcgtagcatcacgttttaatc

SEQ ID NO: 73 - DNA, 50 bp, Artificial sequence
  Synthetic oligonucleotide primer
tattatggatccttatcggagagacttgaataagtttgccatttcaatgg

SEQ ID NO: 74 - DNA, 59 bp, Artificial sequence
  Synthetic oligonucleotide primer
ataatagaattcattaaagaggagaaattaactatgcaaatctacgaaggtaaactaac

SEQ ID NO: 75 - DNA, 37 bp, Artificial sequence
  Synthetic oligonucleotide primer
tattattatagcggccgctcggagagacttgaataag

SEQ ID NO: 76 - DNA, 60 bp, Artificial sequence
  Synthetic oligonucleotide primer
tattattatagcggccgcatggtggtgatggtgatgtcggagagacttgaataagtttgc

SEQ ID NO: 77 - DNA, 72 bp, Artificial sequence
  Synthetic oligonucleotide primer
tattattatagcggccgcgccagaaccgccatggtggtgatggtgatgtcggagagacttgaataagtttgc

SEQ ID NO: 78 - protein, 10 aa, Artificial sequence
  Synthetic peptide sequence
HHHHHHGGSG

SEQ ID NO: 79 - DNA, 30 bp, Artificial sequence
  Synthetic oligonucleotide primer
tattattataaccggtatttcaaatgcgcc

SEQ ID NO: 80 - DNA, 50 bp, Artificial sequence
  Synthetic oligonucleotide primer
ataatagaattcattaaagaggagaaattaactatgcagatctacgaagg

SEQ ID NO: 81 - DNA, 36 bp, Artificial sequence
  Synthetic oligonucleotide primer
tattatggatcctcggagagacttgaataagtttgc

SEQ ID NO: 82 - protein, 24 aa, Artificial sequence
  Linker peptide sequence
SNNNNNNNNNNLGIEGRISEFAAA

SEQ ID NO: 83 - protein, 8 aa, Artificial sequence
  Linker peptide sequence
LAAAGGGG

SEQ ID NO: 84 - protein, 11 aa, Artificial sequence
  Linker peptide sequence
GSVDLQPSLIS

SEQ ID NO: 85 - protein, 13 aa, Artificial sequence
  Biotinylation peptide sequence
LGGIFEAMKMEWR

SEQ ID NO: 86 - protein, 6 aa, Artificial sequence
  Linker peptide sequence
HHHAAA

SEQ ID NO: 87 - protein, 13 aa, Artificial sequence
  Linker peptide sequence
HHHHHHGGSGAAA

SEQ ID NO: 88 - protein, 19 aa, Artificial sequence
  Mink enteritis virus VP2 antigenically peptide
MGDGAVQPDGGQPAVRNER

SEQ ID NO: 89 - protein, 18 aa, Artificial sequence
  Mink enteritis virus VP2 antigenically active peptide
GDGAVQPDGGQPAVRNER

SEQ ID NO: 90 - protein, 9 aa, Artificial sequence
  FLAG peptide sequence
MDYKDDDDK

SEQ ID NO: 91 - protein, 6 aa, Artificial sequence
  His6 peptide sequence
HHHHHH

SEQ ID NO: 92 - protein, 10 aa, Artificial sequence
  Linker peptide sequence
GGGGSGGGSG

SEQ ID NO: 93 - protein, 11 aa, Artificial sequence
  Linker peptide sequence
GGGSGGGSGGG

SEQ ID NO: 94 - protein, 160 aa, Mycobacterium avium
MSPAAGVPEMPALDASGVRLGIVASTWHSRICDALLAGARKVAADSGVENPTVVRVLGAIEIPVVAQELARNHDAVVALGVVIRGQTPHFEYVCDAVTQGITRVSLDASTPVANGVLTTDNEQQALDRAGLPDSAEDKGAQAAGAALSAALTLRELRARS

SEQ ID NO: 95 - protein, 154 aa, Mycobacterium tuberculosis
MPDLPSLDASGVRLAIVASSWHGKICDALLDGARKVAAGCGLDDPTVVRVLGAIEIPVVAQELARNHDAVVALGVVIRGQTPHFDYVCDAVTQGLTRVSLDSSTPIANGVLTTNTEEQALDRAGLPTSAEDKGAQATVAALATALTLRELRAHS

SEQ ID NO: 96 - protein, 163 aa, Corynebacterium ammoniagenes
MSKEGLPEVATIDATGISVAVISATWNADICDRLHERALAHAQQLGAEADGFRVVGALEIPVAVQEAARHYDAVVALGCVIRGGTPHFDYVCDSVTQGLTRIALDTSKPIANGVLTVNTHDQAVDRSGAPGAAEDKGVEAMQAALDTVLQLRNIKERASKRGL

SEQ ID NO: 97 - protein, 155 aa, Chlorobium tepidum
MQVQNIEGSLNASGLKFALVVSRFNDFIGQKLVEGAIDCIVRHGGSADEITVIRCPGAFELPSVTRKAMLSGKYDAIVTLGVIIRGSTPHFDVIAAEATKGIAQVGMEAAIPVSFGVLTTENLEQAIERAGTKAGNKGFDAALAAIEMANLYKQL

SEQ ID NO: 98 - protein, 154 aa, Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 99 - protein, 165 aa, Thermotoga maritima
MKVVQGDYRGEGLKIAVVVPRFNDLVTSKLLEGALDGLKRHGVSDENITVVRIPGSMEAIYTLKRLLDLGVHDAIIVLGAVIRGETYHFNVVANEIGKAVAQFNMTSDIPIVFGVLTTDTLEQALNRAGAKSGNKGFEAAMVAIEMANLRKRLRRDVFESDSNGR

SEQ ID NO: 100 - protein, 154 aa, Bacillus subtilis
MNIIQGNLVGTGLKIGIVVGRFNDFITSKLLSGAEDALLRHGVDTNDIDVAWVPGAFEIPFAAKKMAETKKYDAIITLGTVIRGATTHYDYVCNEAAKGIAQAANTTGVPVIFGIVTTENIEQAIERAGTKAGNKGVDCAVSAIEMANLNRSFE

SEQ ID NO: 101 - protein, 154 aa, Bacillus amyloliquefaciens
MNIIQGNLVGTGLKIGIVVGRFNEFITSKLLSGAEDTLIRHGVESNDIDVAWVPGAFEIPFAAKKMAETKKYDAVITLGTVIRGATTHYDYVCNEAAKGIAQAGTATGVPVIFGIVTTETIEQAIERAGTKAGNKGADCAVSAIEMANLNRSFE

SEQ ID NO: 102 - protein, 153 aa, Actinobacillus pleuropneumoniae
MAKITGNLVATGLKFGIVTARFNDFINDKLLSGAIDTLVRHGAYENDIDTAWVPGAFEIPLVAKKMANSGKYDAVICLGTVIRGSTTHYDYVCNEAAKGIGAVALETGVPVIFGVLTTENIEQAIERAGTKAGNKGSECALGAIEIVNVLKAI

SEQ ID NO: 103 - protein, 155 aa, Streptococcus pneumoniae
MNTYEGNLVANNIKIGIVVARFNEFITSKLLSGALDNLKRENVNEKDIEVAWVPGAFEIPLIASKMAKSKKYDAIICLGAVIRGNTSHYDYVCSEVSKGIAQISLNSEIPVMFGVLTTDTIEQAIERAGTKAGNKGSECAQGAIEMVNLIRTLDA

SEQ ID NO: 104 - protein, 154 aa, Staphylococcus aureus
MNFEGKLIGKDLKVAIVVSRFNDFITGRLLEGAKDTLIRHDVNEDNIDVAFVPGAFEIPLVAKKLASSGNYDAVITLGCVIRGATSHYDYVCNEVAKGVSKVNDQTNVPVIFGILTTESIEQAVERAGTKAGNKGAEAAVSAIEMANLLKSIKA

SEQ ID NO: 105 - protein, 156 aa, Vibrio cholerae
MKVIEGGFPAPNAKIAIVISRFNSFINESLLSGAIDTLKRHGQISDDNITVVRCPGAVELPLVAQRVAKTGDYDAIVSLGCVIRGGTPHFDYVCSEMNKGLAQVSLEFSIPVAFGVLTVDTIDQAIERAGTKAGNKGAEAALSALEMINVLSEIDS

SEQ ID NO: 106 - protein, 155 aa, Photobacterium phosporeum
MKVIEGAIVAPNAKVAIVIARFNSFINESLLSGALDTLKRQGQVSYDNITIIRCPGAYELPLVAQLTAKSDRYDAIIALGSVIRGGTHFEYVASECNKGLAQVALDYNIPVAFGVLTVDYLEQAIERAGTKAGNKGAEAALMLLEMVNILAQVES

SEQ ID NO: 107 - protein, 158 aa, Shewanella putrefaciens
MNVVQGNIEAKNAKVAIVISRFNSFLVESLLEGALDTLKRFGQVSDENITVVRVPGAVELPLAARRVAASGKFDGIIALGAVIRGGTPHFDFVAGECNKGLAQIALEFDLPVAFGVLTTDTIEQAIERSGTKAGNKGGEAALSLLEMVNVLQQLEQQL

SEQ ID NO: 108 - protein, 144 aa, Photobacterium leiognathi
MKLLKGVDCTSCCIAIVIARFNSFINENLLSGAINALQRKGQVKAENITVIRCPGAYELPLAAQQIAKQGNYDAIIAIGAVIRGGTPHFDFVAGECNKGLAQVALEYQTPVAFGVLTVDSIEQAIERAGTKMGNKGEEAALSAL

SEQ ID NO: 109 - protein, 156 aa, Shigella flexneri
MNIIEANVATPDARVAITIARFNNFINDSLLEGAIDALKRIGQVKDENITVVWVPGAYELPLAAGALAKTGKYDAVIALGTVIRGGTAHFEYVAGGASNGLAHVAQDSEIPVAFGVLTTESIEQAIERAGTKAGNKGAEAALTALEMINVLKAIKA

SEQ ID NO: 110 - protein, 156 aa, Escherichia coli; identical to SEQ ID NO: 109

SEQ ID NO: 111 - protein, 157 aa, Haemophilus influenzae
MKVLEGSVAAPNAKVAVVIARFNSFINESLLEGAIDALKRIGQVKDENITIVRTPGAYELPLVARRLAESKKFDAIVALGTVIRGGTAHFEYVAGEASSGLGKVAMDAEIPVAFGVLTTENIEQAIERAGTKAGNKGAEAALTALEMVNLIQQIDAA

SEQ ID NO: 112 - protein, 156 aa, Dehalospirillum multivorans
MNIVEGKLSLNGDEKVAIINARFNHIITDRLVEGARDAYLRHGGKDENLDLVLVPGAFEIPMALNRLLACSKYDAVCCLGAVIRGSTPHFDYVSAEVTKGVANVALQFAKPVAFGVLTVDSIEQAIERAGSKAGNKGFEAMVTVIELLSLYSALKN

SEQ ID NO: 113 - protein, 156 aa, Helicobacter pylori
MQIIEGKLQLQGNERVAILTSRFNHIITDRLQEGAMDCFKRHGGDEDLLDIVLVPGAYELPFILDKLLESEKYDGVCVLGAIIRGGTPHFDYVSAEATKGIAHAMLKYSMPVSFGVLTTDNIEQAIERAGSKAGNKGFEAMSTLIELLSLCQTLKG

SEQ ID NO: 114 - protein, 155 aa, Deinococcus radiodurans
MQRIEATLLAHDLKFALVSTRWNHLIVDRLVEGAELAFVQHGGKTENLDHFLVPGSYEVPLVARRLAETGRYDAVVCLGAVIKGDTDHYDFVAGGAANGILNTSLHTGVPVAFGVLTTDTVEQALNRAGIKAGNKGGEAVLAMIETANLLKQIER

SEQ ID NO: 115 - protein, 164 aa, Synechocystis sp.
MTVYEGSFTPPARPFRFALVIARFNDLVTEKLLSGCQDCLKRHGIDVDPAGTQVDYIWVPGSFEVPLVTRKLAVSGQYDAIICLGAVIRGQTPHFDFVAGEAAKGIAAIASQTGVPVIFGILTTDTMQQALERAGIKSNHGWGYAMNALEMASLMRAMAPLTEG

SEQ ID NO: 116 - protein, 161 aa, Porphyromonas gingivalis
MATAYHNLSDYDYESVPCGKDLRIGIAVAEWNHNITEPLMKGAIDTLLEHGVSADNIIVQHVPGTFELTYASAYLAEQHEVDAVIAIGCVVRGDTPHFDYICQGVTQGITQLNIDGFVPVIFGVLTTETMLQAEERAGGKHGNKGTEAAVTALKMAGLERI

SEQ ID NO: 117 - protein, 227 aa, Arabidopsis thaliana
MKSLASPPCLRLIPTAHRQLNSRQSSSACYIHGGSSVNKSNNLSFSSSTSGFASPLAVEKELRSSFVQTAAVRHVTGSLIRGEGLRFAIVVARFNEVVTKLLLEGAIETFKKYSVREEDIEVIWVPGSFEIGVVAQNLGKSGKFHAVLCIGAVIRGDTTHYDAVANSAASGVLSASINSGVPCIFGVLTCEDMDQALNRSGGKAGNKGAETALTALEMASLFEHHLK

SEQ ID NO: 118 - protein, 141 aa, Methanococcus jannaschii
MVLMVNLGFVIAEFNRDITYMMEKVAEEHAEFLGATVKYKIVVPGVFDMPLAVKKLLEKDDVDAVVTIGCVIEGETEHDEIVVHNAARKIADLALQYDKPVTLGISGPGMTRLQAQERVDYGKRAVEAAVKMVKRLKALEE

SEQ ID NO: 119 - protein, 143 aa, Archaeoglobus fulgidus
MEKVKLGMVVAEFNRDITYMMEILGKEHAEFLGAEVSEVIRVPGTFDIPIAVKKMLEKGRVDAVVAIGCVIEGETEHDEIVAQHAARKIMDLSLEYGKPVTLGISGPGMGRIAATERVDYAKRAVEAAVKLVKRLKEYDAEGS

SEQ ID NO: 120 - protein, 139 aa, Methanobacterium thermoautotrophicum
MKKVRIGAVVAEFNYDITHMMLELAKEHARFLDAEITRVIAVPGVFDMPLAVKKLLLEDEIDAVITLGAVIEGATDHDQIVVQHASRKIADLALDYDKPVALGISGPGMTRLEAHQRVDYAKRAVEAAVKMYRRLKEDI

SEQ ID NO: 121 - protein, 157 aa, Chlamydia trachomatis
MKPLKGCPVAKDVRVAIVGSCFNSPIADRLVAGAQETFFDFGGDPSSLTIVRVPGAFEIPCAIKKLLSTSGQFHAVVACGVLIQGETSHYEHIADSVAAGVSRLSLDFCLPITFSVITAPNMEAAWERAGIKGPNLGASGMKTALEMASLFSLIGKE

SEQ ID NO: 122 - protein, 169 aa, Saccharomyces cerevisiae
MAVKGLGKPDQVYDGSKIRVGIIHARWNRVIIDALVKGAIERMVSLGVEEKNIIIETVPGSYELPWGTKRFVDRQAKLGKPLDVVIPIGVLIKGSTMHFEYISDSTTHALMNLQEKVDMPVIFGLLTCMTEEQALARAGIDEAHSMHNHGEDWGAAAVEMAVKFGKNAF

SEQ ID NO: 123 - protein, 158 aa, Brucella abortus
MNQSCPNKTSFKIAFIQARWHADIVDEARKSFVAELAAKTGGSVEVEIFDVPGAYEIPLHAKTLARTGRYAAIVGAAFVIDGGIYRHDFVATAVINGMMQVQLETEVPVLSVVLTPHHFHESKEHHDFFHAHFKVKGVEAAHAALQIVSERSRIAALV

SEQ ID NO: 124 - DNA, 72 bp, Artificial sequence
  pNCO-N-BS-LuSy sequence
gaattcattaaagaggagaaattaactatggcggcggcgcgtagctgcgcggccgctatgaatatcatacaa

SEQ ID NO: 125 - protein, 15 aa, Artificial sequence
  pNCO-N-BS-LuSy sequence
MAAARSCAAAMNIIQ

SEQ ID NO: 126 - DNA, 45 bp, Artificial sequence
  pNCO-C-BS-LuSy sequence
cgctcatttgaattagcggccgcaaacagtttaaaaggatcccga

SEQ ID NO: 127 - protein, 15 aa, Artificial sequence
  pNCO-C-BS-LuSy sequence
RSFELAAANSLKGSR

SEQ ID NO: 128 - DNA, 45 bp, Artificial sequence
  pNCO-BS-LuSy-EC-DHFR sequence
cgctcatttgaattagcggccgctggtggaggcggaatgatcagt

SEQ ID NO: 129 - protein, 15 aa, Artificial sequence
  pNCO-BS-LuSy-EC-DHFR sequence
RSFELAAAGGGGMIS

SEQ ID NO: 130 - DNA, 69 bp, Artificial sequence
  pNCO-N-VP2-BS-LuSy sequence
atgggggacggtgctgttcagccggacggtggtcagccggctgttcgtaacgaacgtatgaatatcata

SEQ ID NO: 131 - protein, 23 aa, Artificial sequence
  pNCO-N-VP2-BS-LuSy sequence
MGDGAVQPDGGQPAVRNERMNII

SEQ ID NO: 132 - DNA, 74 bp, Artificial sequence
  pNCO-C-VP2-BS-LuSy sequence
cgttctttcgaaggtgacggtgctgttcagccggacggtggtcagccggctgttcgtaacgaacgttaggatcc

SEQ ID NO: 133 - protein, 22 aa, Artificial sequence
  pNCO-C-VP2-BS-LuSy sequence
RSFEGDGAVQPDGGQPAVRNER

SEQ ID NO: 134 - DNA, 69 bp, Artificial sequence
  pNCO-C-Biotag-BS-LuSy sequence
cgttctttcgaagcggccgcactcggcggcatcttcgaagctatgaagatggagtggcgctaaggatcc

SEQ ID NO: 135 - protein, 20 aa, Artificial sequence
  pNCO-C-Biotag-BS-LuSy sequence
RSFEAAALGGIFEAMKMEWR

SEQ ID NO: 136 - DNA, 54 bp, Artificial sequence
  pNCO-Lys165-BS-LuSy sequence
cgttctttcgaaggtggcggtggttctggtggtggctctggtaaataaggatcc

SEQ ID NO: 137 - protein, 15 aa, Artificial sequence
  pNCO-Lys165-BS-LuSy sequence
RSFEGGGGSGGGSGK

SEQ ID NO: 138 - DNA, 60 bp, Artificial sequence
  pNCO-Cys167-BS-LuSy sequence
cgttctttcgaaggtggcggtggttctggtggtggctctggtggtggctgctaaggatcc

SEQ ID NO: 139 - protein, 17 aa, Artificial sequence
  pNCO-Cys167-BS-LuSy sequence
RSFEGGGGSGGGSGGGC

SEQ ID NO: 140 - DNA, 45 bp, Artificial sequence
  pFLAG-MAC-BS-LuSy sequence
atggactacaaggacgacgatgacaaagtcaagcttatgaatatc

SEQ ID NO: 141 - protein, 15 aa, Artificial sequence
  pFLAG-MAC-BS-LuSy sequence
MDYKDDDDKVKLMNI

SEQ ID NO: 142 - DNA, 39 bp, Artificial sequence
  pNCO-C-His6-BS-LuSy sequence
cgttctttcgaacatcaccatcaccaccattaaggatcc

SEQ ID NO: 143 - protein, 10 aa, Artificial sequence
  pNCO-C-His6-BS-LuSy sequence
RSFEHHHHHH

SEQ ID NO: 144 - DNA, 69 bp, Artificial sequence
  pNCO-C-Biotag-AA-LuSy sequence
aagtctctccgagcggccgcactcggcggcatcttcgaagctatgaagatggagtggcgctaaggatcc

SEQ ID NO: 145 - protein, 20 aa, Artificial sequence
  pNCO-C-Biotag-AA-LuSy sequence
KSLRAAALGGIFEAMKMEWR

SEQ ID NO: 146 - DNA, 87 bp, Artificial sequence
  pNCO-His6-C-Biotag-AA-LuSy sequence
aagtctctccgacatcaccatcaccaccatgcggccgcactcggcggcatcttcgaagctatgaagatggagtggcgctaaggatcc

SEQ ID NO: 147 - protein, 26 aa, Artificial sequence
  pNCO-His6-C-Biotag-AA-LuSy sequence
KSLRHHHHHHAAALGGIFEAMKMEWR

SEQ ID NO: 148 - DNA, 99 bp, Artificial sequence
  pNCO-His6-Gly2-Ser-Gly-C-Biotag-AA-LuSy sequence
aagtctctccgacatcaccatcaccaccatggcggttctggcgcggccgcactcggcggcatcttcgaagctatgaagatggagtggcgctaaggatcc

SEQ ID NO: 149 - protein, 30 aa, Artificial sequence
  pNCO-His6-Gly2-Ser-Gly-C-Biotag-AA-LuSy sequence
KSLRHHHHHHGGSGAAALGGIFEAMKMEWR

SEQ ID NO: 150 - DNA, 45 bp, Artificial sequence
  pNCO-AA-BglII-LuSy sequence
gaattcattaaagaggagaaattaactatgcagatctacgaaggt

SEQ ID NO: 151 - protein, 6 aa, Artificial sequence
  pNCO-AA-BglII-LuSy sequence
MQIYEG

SEQ ID NO: 152 - DNA, 48 bp, Artificial sequence
  pNCO-AA-BglII-LuSy-(BamHI) sequence
aagtctctccgaggatccgtcgacctgcagccaagcttaattagctga

SEQ ID NO: 153 - protein, 15 aa, Artificial sequence
  pNCO-AA-BglII-LuSy-(BamHI) sequence
KSLRGSVDLQPSLIS

SEQ ID NO: 154 - protein, 11 aa, Artificial sequence
  GSVDLQPSLIS-lumazine synthase fusion protein sequence
GSVDLQPSLIS